(19) United States
(12) Patent Application Publication
(10) Pub. No.: US 2005/0287625 A1
(43) Pub. Date: Dec. 29, 2005

(54) Process for expression of foreign genes in methylotrophic bacteria through chromosomal integration

(76) Inventors: Edward S. Miller JR., Wilmington, DE (US); Rick W. Ye, Hockessin, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/070,080

(22) Filed: Mar. 2, 2005

Related U.S. Application Data
(60) Provisional application No. 60/550,385, filed on Mar. 5, 2004.

(51) Int. Cl. C12P 23/00; C12N 9/00; C12N 15/74; C12N 1/21

(52) U.S. Cl. 435/67; 435/252.3; 435/471

(57) ABSTRACT

Provided is a method for expressing an introduced gene or genes in a C1 metabolizing microorganism host wherein the gene(s) are integrated into the tig region of the chromosome. This method provides high level expression in a stable manner in which growth rate of the host strain is not highly affected and a selection marker is not required. The use of this method for expressing carotenoid biosynthetic genes and resulting production of canthaxanthin is also described.

This application claims the benefit of U.S. Provisional Application No. 60/550,385 filed Mar. 5, 2004.

### FIELD OF INVENTION

The present invention relates to bacterial gene expression and metabolic engineering. More specifically, this invention relates to methods for the expression of introduced genes in methane-utilizing bacteria through random integration in the chromosome with color selection with gene clusters involved in carotenoid biosynthesis

### BACKGROUND OF THE INVENTION

There are a number of microorganisms that utilize single carbon substrates as their sole energy source. Such microorganisms are referred to herein as “C1 metabolizers”. These organisms are characterized by the ability to use carbon substrates lacking carbon to carbon bonds as a sole source of energy and biomass. All C1 metabolizing microorganisms are generally classified as methylotrophs. Methylotrophs may be defined as any organism capable of oxidizing organic compounds that do not contain carbon-carbon bonds. Methanotrophic bacteria are a type of methylotrophs and are defined by their ability to use methane as their sole source of carbon and energy under ambient conditions. This ability, in conjunction with the abundance of methane, makes the biotransformation of methane a potentially unique and valuable process. As such, several approaches have been used in attempts to harness the unique natural abilities of these organisms for commercial applications.

Historically, the commercial applications of biotransformation of methane have fallen broadly into three categories:
1) Production of single cell protein (Sharpe D. H. BioProtein Manufacture (1989). Ellis Horwood series in applied science and industrial technology. New York: Halstead Press) (Villadsen, John, Recent Trends Chem. React. Eng., [Proc. Int. Chem. React. Eng. Conf.], 2nd (1987), Volume 2, pp 320-33. Editor(s): Kulkarni, B. D.; Mashelkar, R. A.; Sharma, M. M. Publisher: Wiley East., New Delhi, India; Naguib, M., Proc. OAPEC Symp. Petroprotein, [Pap.] (1980), Meeting Date 1979, pp 253-77 Publisher: Organ. Arab Pet. Exporting Countries, Kuwait, Kuwait);
2) Epoxidation of alkenes for production of chemicals (U.S. Pat. No. 4,348,476); and
3) Biodegradation of chlorinated pollutants (Tsien et al., Gas, Oil, Coal, Environ. Biotechnol. 2, [Pap. Int. IGT Symp. Gas, Oil, Coal, Environ. Biotechnol.], 2nd (1990), pp 83-104. Editor(s): Akin, Cavit; Smith, Jared. Publisher: Inst. Gas Technol., Chicago, Ill.; WO 9,633,821; Merkley et al., Biorem. Recalcitrant Org., [Pap. Int. In Situ On-Site Bioreclam. Symp.], 3rd (1995), pp 165-74. Editor(s): Hinchee, Robert E; Anderson, Daniel B.; Hoeppel, Ronald E. Publisher: Battelle Press, Columbus, Ohio; Meyer et al., Microb. Releases 2(1): 11-22 (1993)).

Epoxidation of alkenes has experienced only slight commercial success due to low product yields, toxicity of products and the large amount of cell mass required to generate products.

Large-scale protein production from methane, termed single cell protein or SCP, has been technically feasible and commercialized at large scale (Villadsen, supra). Single cell protein is a relatively low value product. As such, the economic production cannot tolerate heavy bioprocessing costs. The yield of the methanotrophic strain used for producing SCP may be critical to the overall economic viability of the process. Microbial biomass produced by methanotrophic bacteria is typically very high in protein content (˜70-80% by weight), which can restrict the direct use of this protein to certain types of animal feed.

In addition to the synthesis of SCP, methanotrophic cells can further build the oxidation products of methane (i.e. methanol and formaldehyde) into complex molecules such as carbohydrates and lipids. For example, under certain conditions methanotrophs are known to produce exopolysaccharides (WO 02/20797, corresponding to U.S. Pat. No. 6,537,786; WO 02/20728, corresponding to U.S. Pat. No. 6,689,601; Ivanova et al., Mikrobiologiya 57(4):600-5 (1988); Kilbane, John J., II Gas, Oil, Coal, Environ. Biotechnol. 3, [Pap. IGT's Int. Symp.], 3rd (1991), Meeting Date 1990, pp 207-26. Editor(s): Akin, Cavit; Smith, Jared. Publisher: IGT, Chicago, Ill.). Similarly, methanotrophs are known to accumulate both isoprenoid compounds and carotenoid pigments of various carbon lengths (WO 02/20733, corresponding to U.S. Pat. No. 6,660,507; WO 02/20728; Urakami et al., J. Gen. Appl. Microbiol. 32(4):317-41 (1986)).

Most recently, the natural abilities of methanotrophic organisms have been stretched by the advances of genetic engineering. Odom et al. have investigated Methyolomonas sp. 16a as a microbial platform of choice for production of a variety of materials beyond single cell protein including carbohydrates, pigments, terpenoid compounds and aromatic compounds (WO 02/20728; WO 02/18617, corresponding to U.S. Ser. No. 09/941,947). This particular pink-pigmented methanotrophic bacterial strain is capable of efficiently using either methanol or methane as a carbon substrate, is metabolically versatile in that it contains multiple pathways for the incorporation of carbon from formaldehyde into 3-carbon units, and is amenable to genetic engineering via bacterial conjugation using donor species such as Escherichia coli. Thus, Methyolomonas sp. 16a can be engineered to produce new classes of products other than those naturally produced from methane.

Further advancement in the metabolic engineering of methanotrophs such as Methyolomonas sp. 16a for production of commercial products, however, is currently limited by the lack of systems for expressing introduced genes that are amenable to large scale growth such as in a bioreactor. Large scale growth for commercial production is best achieved when no selection is required to maintain the presence of the introduced gene. In particular the presence of antibiotic resistance genes is undesirable, in terms of required regulatory approvals and cost. Thus the first criterion is that the introduced gene must be stably maintained in the host without the presence of an antibiotic resistance gene and use of an antibiotic in the growth medium. Metabolic engineering has in general been accomplished through the introduction of a coding region(s) as part of a chimeric gene(s) on a replicating plasmid. Maintenance of the plasmid within a host requires a selection pressure, typically an antibiotic resistance gene expressed from the plasmid and the antibiotic supplied in the growth medium. Nutritional selection markers may also be used, but these generally decrease the growth rate of the host cells. The presence of the plasmid itself also generally decreases the growth rate of the host cells due to the extra load on the cell's metabolism. Alternatively, introduced coding regions may be integrated into the host chromosome. If the integrated coding regions have low expression levels they are inadequate for production of a commercial product. Thus, a second criterion is that the introduced coding region must be expressed at a high enough level to adequately confer the ability to produce the desired product. A third criterion is that the growth rate of the host organism should not be compromised. As stated above, plasmid expression systems generally lead to a reduced growth rate of the host due to the presence of the plasmid and/or to the selection system. The problem to be solved, therefore, is to develop an expression system that satisfies these criteria.

Applicant has solved the problem by identifying a region of a Methylotroph genome where a coding region(s) can be introduced providing stable maintenance of the insertion without selection, high expression of the introduced coding region, with only a moderate decrease in the host's original growth rate. This genomic region was identified through screening of random insertions of the canthaxanthin gene cluster, which provides a color selection if expressed.

### SUMMARY OF THE INVENTION

The invention relates the discovery that the tig region of the genome in a C1 metabolizing microorganism is an effective point of integration for the high level expression of foreign genes. A gene cluster encoding elements of the lower carotenoid biosynthetic pathway was introduced into the tig region resulting in high level, and more importantly, stable production of C40 carotenoids. Although the invention is exemplified by the integration and expression of a the genes in the lower carotenoid biosynthetic pathway the skilled artisan will readily understand that any foreign gene or gene cluster will perform in substantially the same way.

Accordingly, it is an object of the present invention to provide . . . . a method for over expressing a nucleic acid molecule in a C1 metabolizing microorganism comprising:
a) providing a C1 metabolizing microorganism having a tig region in the genome;
b) providing at least one nucleic acid molecule to be over-expressed
c) integrating the at least one nucleic acid molecule of (b) into said tig region of the genome of said C1 metabolizing microorganism; and
d) growing the C1 metabolizing microorganism of c) under conditions whereby the at least one nucleic acid molecule is over-expressed.

In another embodiment the invention provides a method for the production of a carotenoid compound comprising:
a) providing a C1 metabolizing microorganism comprising a gene cluster comprising genes encoding the carotenoid biosynthetic pathway operably inserted into the tig region of the genome;
b) contacting the C1 metabolizing microorganism of (a) with a C1 carbon substrate selected from the group consisting of methane and methanol under conditions where said gene cluster is expressed and at least one carotenoid compound is produced; and
c) optionally recovering said carotenoid compound of (b)

In an alternate embodiment the invention provides a C1 metabolizing microorganism comprising at least one nucleic acid molecule integrated in the tig region of the genome.

### BRIEF DESCRIPTION OF THE FIGURES, SEQUENCE DESCRIPTIONS AND BIOLOGICAL DEPOSITS

FIG. 1 shows the upper isoprenoid and lower carotenoid biosynthetic pathways.

FIG. 2 shows the gene structure of the tig region of Methylomonas sp. 16a and the integration site identified by screening (triangle).

FIG. 3 is a plasmid map of pGP704.

FIG. 4 shows plasmid maps of constructions for integration vectors.

FIG. 5 shows the plasmid maps of the double-crossover integration vectors.

FIG. 6 shows the production of canthaxanthin by strain Tig333-16 using HPLC analysis.

FIG. 7 shows the fermentation profile of the Methylomonas sp. 16a TigG333-16 strain; lower curve: canthaxanthin; upper curve: cell counts.

FIG. 8 shows canthaxanthin intermediates produced during fermentation of strain Tig333-16, analyzed by HPLC.

FIG. 9 shows canthaxanthin isomers produced during fermentation of strain Tig333-16, analyzed by HPLC.

The invention can be more fully understood from the following detailed description and the accompanying sequence descriptions, which form a part of this application.

The following sequences conform with 37 C.F.R. 1.821-1.825 (“Requirements for Patent Applications Containing Nucleotide Sequences and/or Amino Acid Sequence Disclosures-the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is the nucleotide sequence of the tig region of Methylomonas sp. 16a.

SEQ ID NO:2 is the nucleotide sequence of the crtN1 gene from Methylomonas sp. 16a.

SEQ ID NO:3 is the nucleotide sequence of the ald gene from Methylomonas sp. 16a.

SEQ ID NO:4 is the nucleotide sequence of the crtN2 gene from the Methylomonas sp. 16a. crtN1aldN2 gene cluster of Methylomonas.

SEQ ID NO:5 is the nucleotide sequence of the crtN3 gene from Methylomonas sp. 16a.

SEQ ID NO:6 is the nucleotide sequence of the crtE-idi-crtY-crtI-crtB gene cluster from Pantoea agglomerans.

SEQ ID NO:7 is the nucleotide sequence of the codon-optimized β3-carotene ketolase gene from Agrobacterium aurantiacum.

SEQ ID NO:8 is the nucleotide sequence of the wild-type β-carotene ketolase gene from Agrobacterium aurantiacum.

SEQ ID NOs:9 and 10 are the nucleotide sequences of primers DrdI/npr-sacB and TthIII/npr-sacB, respectively, used for amplification of the npr-sacB cassette from plasmid pBE83, as described in Example 2.

NEW SEQ ID NO:11 is the nucleotide sequence of the Methylomonas tig gene.

NEW SEQ ID NO:12 is the amino acid sequence of the protein encoded by the Methylomonas tig gene.

NEW SEQ ID NO:13 is the nucleotide sequence of the Methylomonas clpP gene.

NEW SEQ ID NO:14 is the amino acid sequence of the protein encoded by the Methylomonas clpP gene.

NEW SEQ ID NO:15 is the nucleotide sequence of the Methylomonas clpX gene.

NEW SEQ ID NO:16 is the amino acid sequence of the protein encoded by the Methylomonas clpX gene.

NEW SEQ ID NO:17 is the nucleotide sequence of the Methylomonas Ion gene.

NEW SEQ ID NO:18 is the amino acid sequence of the protein encoded by the Methylomonas Ion gene.

NEW SEQ ID NO:19 is the nucleotide sequence of the Methylomonas himA gene.

NEW SEQ ID NO:20 is the amino acid sequence of the protein encoded by the Methylomonas himA gene.

NEW SEQ ID NO:21 is the nucleotide sequence of the Methylomonas ppiC gene.

NEW SEQ ID NO:22 is the amino acid sequence of the protein encoded by the Methylomonas ppiC gene.

SEQ ID NOs:23-32 are the nucleotide sequences of primers used for cloning of the carotenoid deletion fragments, as described in Example 3.

SEQ ID NO:33 is the amino acid sequence of the β-carotene ketolase enzyme from Agrobacterium aurantiacum.

SEQ ID NO:34 is the nucleotide sequence of a primer used in a single-primer amplification procedure to amplify the chromosomal DNA sequence adjacent to the crtEWYIB insertion.

SEQ ID NO:35 is the nucleotide sequence of the crtE gene from Pantoea stewartii.

SEQ ID NO:36 is the nucleotide sequence of the crtYIB gene cluster from Pantoea stewartii.

SEQ ID NOs: 37-40 are the nucleotide sequences of primers used to construct the canthaxanthin expression plasmid pDCQ307, as described in Example 8.

SEQ ID NO:41 is the nucleotide sequence of the crtEidiYIBZ gene cluster from Pantoea agglomerans.

SEQ ID NOs:42 and 43 are the nucleotide sequences of primers used to construct the canthaxanthin expression plasmid pDCQ333, as described in Example 11.

SEQ ID NOs:44 and 45 are the nucleotide sequences of a linker used in the construction of the integration vector in Example 7.

SEQ ID NOs:46 and 47 are the nucleotide sequences of primers used to amplify the kanamycin gene from plasmid pBHR1 described in Example 7.

SEQ ID NOs:48 and 49 are the nucleotide sequences of primers used to amplify the npr-sacB gene from pGP704::sacB described in Example 7.

SEQ ID NOs:50 and 51 are the nucleotide sequences of primers used to amplify the origin of replication from pACYC described in Example 7.

SEQ ID NO:34 is the nucleotide sequence of a primer used in a single-primer amplification procedure to amplify the chromosomal DNA sequence adjacent to the crtEWYIB insertion.

SEQ ID NOs:52 and 53 are the nucleotide sequences of primers used to sequence the genomic DNA fragments obtained with primer SEQ ID NO:34.

SEQ ID NOs:54 and 55 are the nucleotide sequences of primers used to amplify a 1 kB region of the tig gene to be used as an integration homology region as described in Example 11.

SEQ ID NOs:56 and 57 are the nucleotide sequences of primers used to amplify a 1.4 kB region of the clpP-clpX genes to be used as an integration homology region as described in Example 11.

SEQ ID NO:58 is the nucleotide sequence of a primer used together with SEQ ID NO:34 to confirm single-crossover integration as described in Example 12.

SEQ ID NOs:59 and 60 are the nucleotide sequences of primers used to confirm double-crossover integration as described in Example 12.

The following biological deposit was made under the terms of the Budapest Treaty on the International Recognition of the Deposit of Micro-organisms for the Purposes of Patent Procedure:

| Depositor | International | |
|---|---|---|
| Identification | Depository | |
| Reference | Designation | Date of Deposit |
| Methylomonas 16a | ATCC PTA 2402 | Aug. 22, 2000 |

As used herein, “ATCC” refers to the American Type Culture Collection International Depository Authority located at ATCC, 10801 University Blvd., Manassas, Va. 20110-2209, USA. The “International Depository Designation” is the accession number to the culture on deposit with ATCC.

The listed deposit will be maintained in the indicated international depository for at least thirty (30) years and will be made available to the public upon the grant of a patent disclosing it. The availability of a deposit does not constitute a license to practice the subject invention in derogation of patent rights granted by government action.

### DETAILED DESCRIPTION OF THE INVENTION

The present invention relates to the finding that the tig region of the genome of C1 metabolizing organisms is an opportune location for the integration and overexpression of foreign genes from these host cells. In particular it has been discovered that a gene cluster encoding the enzymes of the lower carotenoid pathway, when inserted in this region, stably produce high levels of C₄₀ carotenoids (e.g. canthaxanthin).

There is a general practical utility for microbial production of C₄₀ carotenoid compounds. This practical utility results since these compounds are very difficult to make chemically (Nelis and Leenheer, Appl. Bacteriol. 70:181-191 (1991)). Industrially, only a few carotenoids are used for food colors, animal feeds, pharmaceuticals, and cosmetics, despite the existence of more than 600 different carotenoids identified in nature. Most carotenoids have strong color and can be viewed as natural pigments or colorants. Furthermore, many carotenoids have potent antioxidant properties and thus inclusion of these compounds in the diet is thought to provide health benefits. Carotenoids produced in a microbial host may be used as a part of the single cell protein product, or may be purified prior to use.

Most preferred is use of the tig region integration system for expression of the crtEWYIB and crtWEidiYIB gene clusters in Methylomonas sp. 16a MWM1200, providing host strains for commercial production of the carotenoid canthaxanthin. Canthaxanthin is used, for example, in fish and poultry feed to impart a pink or orange color to the flesh. The all-E isomer of canthaxanthin, as opposed to the Z isomers, is required in a commercial feed product. Only the all-E isomer is absorbed across the mixed micelia in the fish intestine and is taken up into the fish muscle. The Z isomers do not cross the mixed micelia and remain unabsorbed.

Definitions

In this disclosure, a number of terms and abbreviations are used. The following definitions are provided.
“Open reading frame” is abbreviated ORF.
“Polymerase chain reaction” is abbreviated PCR.
“High Performance Liquid Chromatography” is abbreviated HPLC.
“Kanamycin” is abbreviated Kan.
“Ampicillin” is abbreviated Amp.

The term “isoprenoid compound” refers to compounds formally derived from isoprene (2-methylbuta-1,3-diene; CH₂═C(CH₃)CH═CH₂), the skeleton of which can generally be discerned in repeated occurrence in the molecule. These compounds are produced biosynthetically via the isoprenoid pathway beginning with isopentenyl pyrophosphate (IPP) and formed by the head-to-tail condensation of isoprene units, leading to molecules which may be, for example, of 5, 10, 15, 20, 30, or 40 carbons in length.

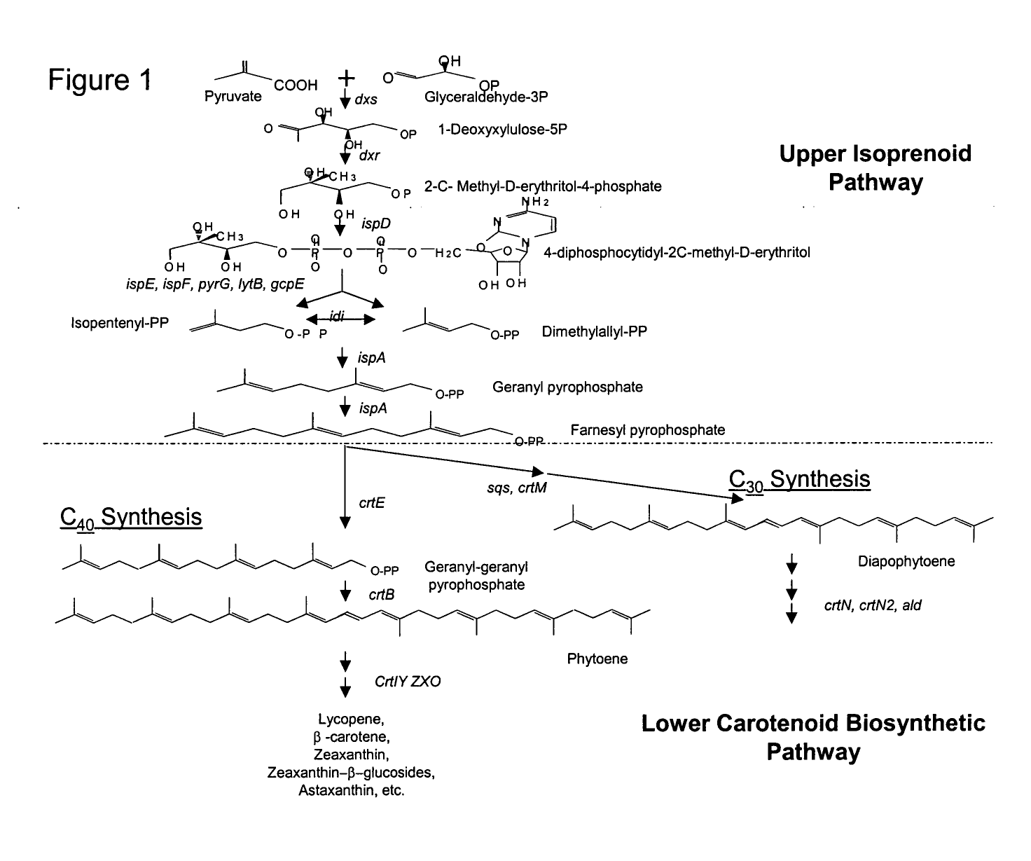

The term “carotenoid biosynthetic pathway” refers to those genes comprising members of the upper isoprenoid pathway and/or lower carotenoid biosynthetic pathway, as illustrated in FIG. 1.

The terms “upper isoprenoid pathway” and “upper pathway” are used interchangeably and refer to enzymes involved in converting pyruvate and glyceraldehyde-3-phosphate to farnesyl pyrophosphate (FPP). Genes encoding these enzymes include, but are not limited to: the “dxs”gene (encoding 1-deoxyxylulose-5-phosphate synthase); the “dxr”gene (encoding 1-deoxyxylulose-5-phosphate reductoisomerase); the “ispD” gene (encoding a 2C-methyl-D-erythritol cytidyltransferase enzyme; also known as ygbP); the “ispE” gene (encoding 4-diphosphocytidyl-2-C-methylerythritol kinase; also known as ychB); the “ispF”gene (encoding a 2C-methyl-D-erythritol 2,4-cyclodiphosphate synthase; also known as ygbB); the “pyrG”gene (encoding a CTP synthase); the “lytB” gene involved in the formation of dimethylallyl diphosphate; the “gcpE” gene involved in the synthesis of 2-C-methyl-D-erythritol 4-phosphate; the “idi” gene (responsible for the intramolecular conversion of IPP to dimethylallyl pyrophosphate); and the “ispA”gene (encoding geranyltransferase or farnesyl diphosphate synthase) in the isoprenoid.

The terms “lower carotenoid biosynthetic pathway” and “lower pathway” will be used interchangeably and refer to those enzymes which convert FPP to a suite of carotenoids. These include those genes and gene products that are involved in the immediate synthesis of either diapophytoene (whose synthesis represents the first step unique to biosynthesis of C₃₀ carotenoids) or phytoene (whose synthesis represents the first step unique to biosynthesis of C₄₀ carotenoids). All subsequent reactions leading to the production of various C₃₀-C₄₀ carotenoids are included within the lower carotenoid biosynthetic pathway. These genes and gene products comprise all of the “crt” genes including, but not limited to: crtM, crtN1, crtN2, crtE, crtX, crtY, crtI, crtB, crtZ, crtW, crtR, crtL, crtO, crtA, crtC, crtD, crtF, and crtU. Finally, the term “lower carotenoid biosynthetic enzyme” is an inclusive term referring to any and all of the enzymes in the present lower pathway including, but not limited to: CrtM, CrtN, CrtN2, CrtE, CrtX, CrtY, CrtI, CrtB, CrtZ, CrtW, CrtR, CrtL, CrtO, CrtA, CrtC, CrtD, CrtF, and CrtU.

The term “carotenoid” refers to a class of hydrocarbons having a conjugated polyene carbon skeleton formally derived from isoprene. This class of molecules is composed of C₃₀ diapocarotenoids and C₄₀ carotenoids and their oxygenated derivatives; and, these molecules typically have strong light absorbing properties.

“C₃₀ diapocarotenoids” consist of six isoprenoid units joined in such a manner that the arrangement of isoprenoid units is reversed at the center of the molecule so that the two central methyl groups are in a 1,6-positional relationship and the remaining nonterminal methyl groups are in a 1,5-positional relationship. All C₃₀ carotenoids may be formally derived from the acyclic C₃₀H₄₂ structure (Formula I below, hereinafter referred to as “diapophytoene”), having a long central chain of conjugated double bonds, by: (i) hydrogenation (ii) dehydrogenation, (iii) cyclization, (iv) oxidation, (v) esterification/glycosylation, or any combination of these processes.

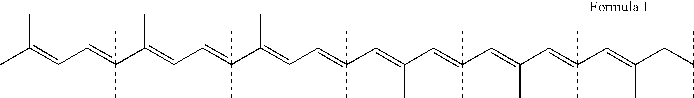

“Tetraterpenes” or “C₄₀ carotenoids” consist of eight isoprenoid units joined in such a manner that the arrangement of isoprenoid units is reversed at the center of the molecule so that the two central methyl groups are in a 1,6-positional relationship and the remaining nonterminal methyl groups are in a 1,5-positional relationship. All C₄₀ carotenoids may be formally derived from the acyclic C₄₀H₅₆ structure. Non-limiting examples of C₄₀ carotenoids include: phytoene, lycopene, 1-carotene, zeaxanthin, astaxanthin, and canthaxantin.

The term “CrtE” refers to a geranylgeranyl pyrophosphate synthase enzyme encoded by the crtE gene and which converts trans-trans-farnesyl diphosphate and isopentenyl diphosphate to pyrophosphate and geranylgeranyl diphosphate.

The term “Idi” refers to an isopentenyl diphosphate isomerase enzyme (E.C. 5.3.3.2) encoded by the idi gene.

The term “CrtY” refers to a lycopene cyclase enzyme encoded by the crtY gene which converts lycopene to β-carotene.

The term “CrtI” refers to a phytoene desaturase enzyme encoded by the crtI gene. CrtI converts phytoene into lycopene via the intermediaries of phytofluene, ζ-carotene and neurosporene by the introduction of 4 double bonds.

The term “CrtB” refers to a phytoene synthase enzyme encoded by the crtB gene which catalyzes the reaction from prephytoene diphosphate to phytoene.

The term “CrtZ” refers to a carotenoid hydroxylase enzyme (e.g. β-carotene hydroxylase) encoded by the crtZ gene which catalyzes a hydroxylation reaction. The oxidation reaction adds a hydroxyl group to cyclic carotenoids having a α-ionone type ring. This reaction converts cyclic carotenoids, such as α-carotene or canthaxanthin, into the hydroxylated carotenoids zeaxanthin or astaxanthin, respectively. Intermediates in the process typically include β-cryptoxanthin and adonirubin. It is known that CrtZ hydroxylases typically exhibit substrate flexibility, enabling production of a variety of hydroxylated carotenoids depending upon the available substrates. The term “CrtW” refers to a α-carotene ketolase enzyme encoded by the crtW gene that catalyzes an oxidation reaction where a keto group is introduced on the β-ionone type ring of cyclic carotenoids. The term “carotenoid ketolase” or “ketolase” refers to the group of enzymes that can add keto groups to the ionone type ring of cyclic carotenoids.

The term “CrtX” refers to a zeaxanthin glucosyl transferase enzyme encoded by the crtX gene and which converts zeaxanthin to zeaxanthin-β-diglucoside.

The term “crt gene cluster” refers to a tandomly arrayed group of genes that encode proteins involved in carotenoid biosynthesis. All of the genes in a gene cluster are transcribed from the same promoter.

The term “crtE-idi-crtY-crtI-crtB” or “crtEidiYIB” gene cluster refers to a DNA segment having the following genetic organization: the crtE, idi, crtY, crtI, and crtB genes are clustered in the order stated.

The term “C₁ carbon substrate” refers to any carbon-containing molecule that lacks a carbon-carbon bond. Non-limiting examples are methane, methanol, formaldehyde, formic acid, formate, methylated amines (e.g., mono-, di-, and tri-methyl amine), methylated thiols, and carbon dioxide. In one embodiment, the C₁ carbon substrate is methanol and/or methane.

The term “C₁ metabolizer” refers to a microorganism that has the ability to use a single carbon substrate as its sole source of energy and biomass. C₁ metabolizers will typically be methylotrophs and/or methanotrophs.

The term “C₁ metabolizing bacteria” or “C₁ metabolizing microorganism” refers to bacteria that have the ability to use a single carbon substrate as their sole source of energy and biomass. C₁ metabolizing bacteria, a subset of C₁ metabolizers, will typically be methylotrophs and/or methanotrophs.

The term “methylotroph” means an organism capable of oxidizing organic compounds that do not contain carbon-carbon bonds. Where the methylotroph is able to oxidize CH₄, the methylotroph is also a methanotroph. In one embodiment, the methylotroph utilizes methanol and/or methane as a primary carbon source. In another embodiment, the methylotroph is a methanotroph utilizing methanol and/or methane as a primary carbon source.

The term “methanotroph” or “methanotrophic bacteria” means a prokaryote capable of utilizing methane as its primary source of carbon and energy. Complete oxidation of methane to carbon dioxide occurs by aerobic degradation pathways. Typical examples of methanotrophs useful in the present invention include (but are not limited to) the genera Methylomonas, Methylobacter, Methylococcus, and Methylosinus.

The term “high growth methanotrophic bacterial strain” refers to a bacterium capable of growth with methane or methanol as the sole carbon and energy source and which possesses a functional Embden-Meyerhof carbon flux pathway, resulting in a high rate of growth and yield of cell mass per gram of C₁ substrate metabolized (see WO 02/20728; corresponding to U.S. Pat. No. 6,689,601, hereby incorporated by reference). The specific “high growth methanotrophic bacterial strain” described herein is referred to as “Methylomonas 16a”, “16a” or “Methylomonas sp. 16a”, which terms are used interchangeably and which refer to the Methylomonas strain used in the present invention.

The term “CrtN1” refers to an enzyme encoded by the crtN1 gene, active in the native carotenoid biosynthetic pathway of Methylomonas sp. 16a. This gene is located within an operon comprising crtN2 and ald.

The term “ALD” refers to an enzyme encoded by the ald gene, active in the native carotenoid biosynthetic pathway of Methylomonas sp. 16a. This gene is located within an operon comprising crtN1 and crtN2.

The term “CrtN2” refers to an enzyme encoded by the crtN2 gene, active in the native carotenoid biosynthetic pathway of Methylomonas sp. 16a. This gene is located within an operon comprising crtN1 and ald.

The term “CrtN3” refers to an enzyme encoded by the crtN3 gene, active in the native carotenoid biosynthetic pathway of Methylomonas sp. 16a. This gene is not located within the crtN1aldcrtN2 gene cluster; instead this gene is present in a different location within the Methylomonas genome.

The term “Sqs” refers to the squalene dehydrogenase enzyme encoded by the sqs gene.

The term “pigmentless” or “white mutant” refers to a Methylomonas sp. 16a bacterium wherein the native pink pigment (e.g., a C₃₀ carotenoid) is not produced (U.S. Ser. No. 10/997,844, hereby incorporated by reference). Thus, the bacterial cells appear white in color, as opposed to pink.

The term “stably-expressed” as it applies to the integration of a nucleic acid molecule into the tig region of a C1 host refers to an integration event that results in the expression of the integrated nucleic acid molecule for for over a hundred generations.

The term “positive selection” means a selection method that enables only those cells that carry a DNA insert integrated at a specific chromosomal location to grow under particular conditions. In contrast, negative selection is based on selection methods whereby only those individuals that do not possess a certain character (e.g., cells that do not carry a DNA insert integrated at a specific chromosomal location) are selected.

The term “homologous recombination” refers to the exchange of DNA fragments between two DNA molecules (during crossover). The fragments that are exchanged are flanked by sites of identical nucleotide sequences between the two DNA molecules (i.e., homology DNA regions). Homologous recombination is the most common means for generated genetic diversity in microbes.

The term “chromosomal integration” means that a DNA segment introduced into the cell becomes congruent with the chromosome of a microorganism through recombination between homologous DNA regions on the introduced DNA segment and within the chromosome.

The term “operably inserted” means that the gene or genes that are integrated into a chromosomal region are organized in a manner in which the encoded proteins are expressed from those genes, and the proteins are functional. In general, operable insertion requires that the integrated gene be in the same orientation as any other genes in the same operon.

The term “chromosomal integration vector” means an extra-chromosomal vector that is capable of integrating into the host's genome through homologous recombination.

The term “suicide vector” or “positive selection vector” refers to a type of chromosomal integration vector that is capable of replicating in one host but not in another. Thus, the vector is conditional for its replication.

The terms “single-crossover event” and “plasmid integration” are used interchangeably and mean the incorporation of a chromosomal integration vector into the genome of a host via homologous recombination between regions of homology between DNA present within the chromosomal integration vector and the host's chromosomal DNA. A “single-crossover mutant” refers to a cell that has undergone a single-crossover event.

The term “double-crossover event” means the homologous recombination between a DNA region within the chromosomal integration vector and a region within the chromosome that results in the replacement of the chromosomal nucleotide sequence of interest (i.e., chr-NSI) with a homologous plasmid region. The double-crossover event may be generated by two simultaneous reciprocal breakage and reunion events between the same two DNA fragments; alternatively, a double-crossover event can be the result of two single-crossovers that occur non-simultaneously.

The term “allelic exchange” means the replacement of the chromosomal nucleotide sequence of interest with an integration vector-born homologous DNA sequence that has been modified. This “replacement nucleotide sequence of interest” or “re-NSI” in the integration vector is modified with respect to chr-NSI by the addition, deletion, or substitution of at least one nucleotide An “allelic exchange mutant” is the result of a double-crossover event involving the introduction of the modification within the re-NSI into the chromosome.

The term “chromosomal nucleotide sequence of interest” or “chr-NSI” refers to a specific chromosomal sequence that is targeted for homologous recombination.

The term “homology-nucleotide sequence of interest” or “h-NSI” refers to a nucleotide sequence of interest that is cloned into a chromosomal integration vector for the purpose of inducing homologous recombination with a chromosomal sequence. The h-NSI has no modification as compared to the chr-NSI.

The term “marker” means a gene that confers a phenotypic trait that is easily detectable through screening or selection. A marker used in screening is, for example, one whose conferred trait can be visualized. Genes involved in carotenoid production or that encode proteins (i.e. beta-galactosidase, beta-glucuronidase) that convert a colorless compound into a colored compound are examples of this type of marker. A screening marker gene may also be referred to as a reporter gene. A selectable marker is one wherein cells having the marker gene can be distinguished based on growth. For example, an antibiotic resistance marker serves as a useful selectable marker, since it enables detection of cells which are resistant to the antibiotic, when cells are grown on media containing that particular antibiotic.

The term “SacB” means a Bacillus encoded protein that catalyzes the conversion of sucrose into levan, a product that is toxic to most Gram-negative microorganisms. The term “sacB” means a gene that encodes the “SacB” protein.

A “nucleic acid” is a polymeric compound comprised of covalently linked subunits called nucleotides. Nucleic acids include polyribonucleic acid (RNA) and polydeoxyribonucleic acid (DNA), both of which may be single-stranded or double-stranded. DNA includes cDNA, genomic DNA, synthetic DNA, and semi-synthetic DNA.

As used herein, an “isolated nucleic acid molecule” or “fragement” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

A nucleic acid fragment is “hybridizable” to another nucleic acid fragment, such as a cDNA, genomic DNA, or RNA, when a single stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor (1989), particularly Chapter 11 and Table 11.1 therein.

The term “oligonucleotide” refers to a nucleic acid, generally of about at least 18 nucleotides, that is hybridizable to a genomic DNA molecule, a cDNA molecule, or an mRNA molecule.

The term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid molecules that are complementary to the complete sequences as reported in the accompanying Sequence Listing

“Gene” refers to a nucleic acid fragment that expresses a specific protein. It may or may not include regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

A gene that is “expressable ” is one that produces a functional protein product.

“Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines.

“Synthetic genes” can be assembled from oligonucleotide building blocks that are chemically synthesized using procedures known to those skilled in the art. These building blocks are ligated and annealed to form gene segments that are then enzymatically assembled to construct the entire gene. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

As used herein, the term “homolog”, as applied to a gene, means any gene derived from the same or a different microbe having the same function. A homologous gene may have significant sequence similarity.

“Coding sequence” or “coding region of interest” refers to a DNA sequence that codes for a specific amino acid sequence.

The term “codon optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes. Within the context of the present invention genes and DNA coding regions are codon optimized for optimal expression in Methylomonas sp. 16a.

“Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The “3' non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., the coding sequence is under the transcriptional control of the promoter). Coding sequences can be operably linked to regulatory sequences in sense or antisense orientation.

The term “tig promoter” refers to the DNA sequence located 5′ to the coding region for the trigger factor protein, that directs transcription of at least this coding region.

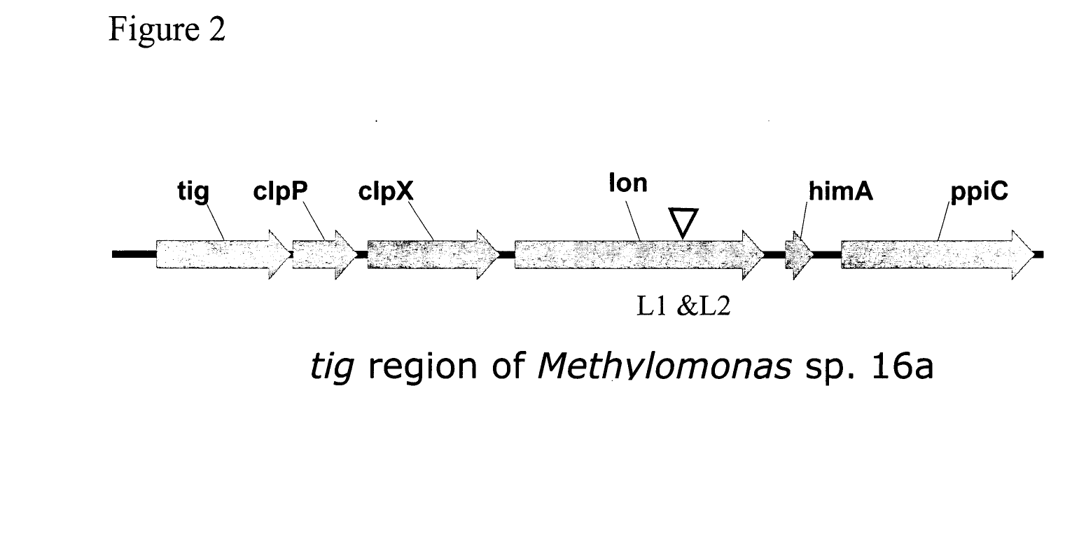

The term “tig region” refers to the region of chromosomal DNA containing coding regions that are all expressed from the tig promoter. The tig region includes the coding region for trigger factor, as well as any other 3′ and adjacent coding regions that do not have promoters, but are transcribed together with the trigger factor coding region (see FIG. 2).

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

“Conjugation” refers to a particular type of transformation in which a unidirectional transfer of DNA (e.g., from a bacterial plasmid) occurs from one bacterium cell (i.e., the “donor”) to another (i.e., the “recipient”). The process involves direct cell-to-cell contact.

The terms “plasmid” and “vector” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a gene or genes into a cell. “Transformation vector” refers to a specific plasmid containing a foreign gene and having elements (in addition to the foreign gene) that facilitate transformation of a particular host cell.

The term “down-regulated” refers to a gene that has been disrupted such that the expression of the gene is less than that associated with the native sequence.

The term “MWM1100 (Δcrt cluster promoter)” refers to a mutant of Methylomonas sp. 16a in which the crt cluster promoter has been disrupted. Disruption of the native C₃₀ carotenoid biosynthetic pathway results in suitable background (pigmentless) for engineering C₄₀ carotenoid production (U.S.10/997,844).

The term “MWM1200 (Δcrt cluster promoter+ΔcrtN3)” refers to a mutant of Methylomonas sp. 16a in which the crt cluster promoter and the crtN3 gene have been disrupted. Disruption of the native C₃₀ carotenoid biosynthetic pathway results in suitable background (pigmentless) for engineering C₄₀ carotenoid production (U.S. Ser. No. 10/997,844).

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: the GCG suite of programs (Wisconsin Package Version 9.0, Genetics Computer Group (GCG), Madison, Wis.); BLASTP, BLASTN, BLASTX (Altschul et al., J. Mol. Biol. 215:403-410 (1990)); DNASTAR (DNASTAR, Inc., Madison, Wis.); and the FASTA program incorporating the Smith-Waterman algorithm (W. R. Pearson, Comput. Methods Genome Res., [Proc. Int. Symp.], Meeting Date 1992,111-20. Suhai, Sandor, Ed.; Plenum: New York, N.Y. (1994)). Within the context of this application it will be understood that where sequence analysis software is used for analysis, the results of the analysis are based on the “default values” of the program referenced, unless otherwise specified. As used herein “default values” will mean any set of values or parameters set by the manufacturer which originally load with the software when first initialized.

Standard recombinant DNA and molecular cloning techniques used here are well known in the art and are described by Sambrook, J., Fritsch, E. F. and Maniatis, T., Molecular Cloning: A Laboratory Manual, 2^{nd} ed., Cold Spring Harbor Laboratory: Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987).

Identification of Integration Region for Stable, High-Level Gene Expression in the Methylomonas Genome

The present invention identifies a region of the Methylomonas genome that provides a location for gene or multiple gene insertion whereby a product that results from expression of those genes is made in high amounts. This region of the genome was identified through screening of Methylomonas sp. 16a cell lines with random insertions of the promoterless crtEWYIB gene cluster in the genome. Random insertion lines producing high levels of the target product were characterized to identify the exact location of the inserted genes. A chromosomal region that we hereby call the tig region was identified as the integration region. Integration of gene(s) in the tig region such that expression is from the tig promoter in a C1 metabolizing microorganism host provides a novel and valuable production platform.

C1 Metabolizing Microorganism Host

All C1 metabolizing microorganisms are generally classified as methylotrophs. Methylotrophs may be defined as any organism capable of oxidizing organic compounds that do not contain carbon-carbon bonds. However, facultative methylotrophs, obligate methylotrophs, and obligate methanotrophs are all various subsets of methylotrophs. Specifically:
Facultative methylotrophs have the ability to oxidize organic compounds which do not contain carbon-carbon bonds, but may also use other carbon substrates such as sugars and complex carbohydrates for energy and biomass;
Obligate methylotrophs are those organisms which are limited to the use of organic compounds that do not contain carbon-carbon bonds for the generation of energy; and
Obligate methanotrophs are those obligate methylotrophs that have the distinct ability to oxidize methane.
   Facultative methylotrophic bacteria are found in many environments, but are isolated most commonly from soil, landfill and waste treatment sites. Many facultative methylotrophs are members of the β, and Γ subgroups of the Proteobacteria (Hanson et al., Microb. Growth C1 Compounds., [Int. Symp.], 7th (1993), 285-302. Editor(s): Murrell, J. Collin; Kelly, Don P. Publisher: Intercept, Andover, UK; Madigan et al., Brock Biology of Microorganisms, 8th edition, Prentice Hall, UpperSaddle River, N.J. (1997)). Facultative methylotrophic bacteria suitable in the present invention include, but are not limited to: Methylophilus, Methylobacillus, Methylobacterium, Hyphomicrobium, Xanthobacter, Bacillus, Paracoccus, Nocardia, Arthrobacter, Rhodopseudomonas, and Pseudomonas.

Those methylotrophs having the additional ability to utilize methane are referred to as methanotrophs. Of particular interest in the present invention are those obligate methanotrophs which are methane utilizers but which are obliged to use organic compounds lacking carbon-carbon bonds. Exemplary organisms included in this classification of obligate methanotrophs that utilize C1 compounds are the genera Methylomonas, Methylobacter, Mehtylococcus, Methylosinus, Methylocyctis, Methylomicrobium, and Methanomonas, although this is not intended to be limiting.

Of particular interest in the present invention are high growth obligate methanotrophs having an energetically favorable carbon flux pathway. For example, a specific strain of methanotroph having several pathway features that makes it particularly useful for carbon flux manipulation is known as Methylomonas 16a (ATCC PTA 2402) (WO 02/20728; corresponding to U.S. Pat. No. 6,689,601). This particular strain and other related methylotrophs including for example, Methylomonas clara and Methylosinus sporium, are preferred microbial hosts for expression of numerous gene products. These strains have both the expected Entner-Douderoff Pathway (which utilizes the keto-deoxy phosphogluconate aldolase enzyme) and in addition, the Embden-Meyerhof Pathway (which utilizes the fructose bisphosphate aldolase enzyme). Energetically, the latter pathway is most favorable and allows greater yield of biologically useful energy, ultimately resulting in greater yield production of cell mass and other cell mass-dependent products.

Strategy for Identification of High Expression Integration Region

It is an object of the invention to provide a C1 metabolizing bacteria capable of stably overexpressing a gene or set of genes for use as a production platform. Integration of the gene or set of genes into the chromosome (as opposed to plasmid expression) affords the best chance for genetic stability, however has drawbacks. For example, integration of an expressible nucleic acid molecule or gene into the chromosome generally results in lower expression than when the same genetic element is present on a replicating plasmid in a host cell. This is to be expected for several reasons including the fact that the integrated gene is present in only one copy (while the gene on the plasmid is present in many copies), and the fact that changes in the regions surrounding the genetic element to be expressed will influence its expression. It was necessary therefore to determine where in the host genome a gene or set of genes would be expressed at the highest levels. The solution to this problem could be approached either rationally (i.e. testing integration sites sequentially) or randomly using a gene or set of genes that would function as a marker if expressed.

The random approach was chosen for it's speed. Genes comprising the lower carotenoid biosynthetic pathway were randomly introduced at a number of sites in the host genome and screened for the production of a carotenoid pigment. It will be appreciated that the same process could be accomplished using more standard markers such as beta-galactosidase, beta-glucuronidase, or other genes that express an enzyme that can metabolize a colorless substrate. In the context of the present invention the carotenoid produced was canthaxanthin, which provided a strong visual marker indicative of expression. In addition, the size of the insert is more than 5 kb and it is useful to search for chromosomal regions that can support a stable expression of a relatively large gene cluster.

Genes for Production of the Carotenoid Canthaxanthin

The synthesis of carotenoids occurs through the upper isoprenoid pathway providing for the conversion of pyruvate and glyceraldehyde-3-phosphate to farnesyl pyrophosphate (FPP) and the lower carotenoid biosynthetic pathway that provides for the synthesis of either diapophytoene (C₃₀) or phytoene (C₄₀) and all subsequently produced carotenoids. Canthaxanthin is a C₄₀ carotenoid.

For the biosynthesis of C₄₀ carotenoids, a series of enzymatic reactions catalyzed by CrtE and CrtB occur to convert FPP to geranylgeranyl pyrophosphate (GGPP) to phytoene, the first 40-carbon molecule of the lower carotenoid biosynthesis pathway. From the compound phytoene, a spectrum of C₄₀ carotenoids are produced by subsequent hydrogenation, dehydrogenation, cyclization, oxidation, or any combination of these processes. Lycopene, which imparts a “red”-colored spectra, is produced from phytoene through four sequential dehydrogenation reactions by the removal of eight atoms of hydrogen, catalyzed by phytoene desaturase (encoded by the gene crtI). Lycopene cyclase (encoded by the gene crtY) converts lycopene to β-carotene. β-carotene can be converted to canthaxanthin by β-carotene ketolases encoded by crtW. Thus the set of genes crtE, crtB, crtI, crtY, and crtW together encode a biosynthetic pathway for the conversion of FPP to canthaxanthin. These genes can be linked together with all coding regions in the same orientation such that expression of one DNA fragment provides for the synthesis of canthaxanthin from FPP. Pantoea stewartii ATCC #8199 (WO 03/016503) contains the natural gene cluster crtEXYIBZ. The crtX gene can be replaced with the crtW gene and the crtZ gene can be deleted to construct a gene cluster for canthaxanthin production.

Methylomonas sp. 16a Strain for Screening Integration Sites

Methylomonas sp. 16a is normally pink in color due to production of C₃₀ carotenoids. For visual screening of canthaxanthin production, C₃₀ carotenoid production can be eliminated in a strain to provide a non-pigmented background.

Two operons have been identified within the Methylomonas sp. 16a genomic sequence containing carotenoid biosynthetic genes. The first biosynthetic operon (referred to herein as the crtN1aldcrtN2 gene cluster), encodes three genes, each of which is described below:
The first gene (designated crtN1; SEQ ID NO:2) encodes a putative diapophytoene dehydrogenase with the highest BLAST hit to a diapophytoene dehydrogenase from Heliobacillus mobilis (34% identity and 58% similarity);
The middle gene (designated ald; SEQ ID NO:3) encodes a putative aldehyde dehydrogenase with the highest BLAST hit to a betaine aldehyde dehydrogenase from Arabidopsis thaliana (33% identity and 50% similarity); and
The third gene (designated crtN2; SEQ ID NO:4) also encodes a putative diapophytoene dehydrogenase with the highest BLAST hit to a hypothetical protein of phytoene dehydrogenase family from Staphylococcus aureus (51% identity and 67% similarity).
   The second biosynthetic operon encodes a fourth gene designated as crtN3 (SEQ ID NO:5). “Clustal W” analysis done to show the relationship between crtN1, crtN2, crtN3 and sqs revealed that crtN3 is not closely linked to crtN1 and crtN2. “Clustal W” is a multiple sequence alignment program for DNA or proteins that produces biologically meaningful multiple sequence alignments of divergent sequences. This program calculates the best match for a selected sequence, and lines them up so that the identities, similarities and differences can be visualized (D. Higgins et. al. Nucleic Acid Res. 22:46734680 (1994)). When the crtN3 (which contains sequences that are homologous to domains of other FAD-dependent oxidoreductases) was viewed in context of its surrounding ORFs, it was observed that crtN3 is located at the end of a cluster of ORFs that have high homology to proteins that play a role in fatty acid metabolism. The crtN3 gene encodes a hypothetical protein with the highest BLAST hit to an unknown conserved protein family from Bacillus halodurans (31% identity and 48% similarity).

Eliminating expression of the crtN1aldcrtN2 gene cluster, as well as crtN3 results in a Methylomonas sp. 16a strain that lacks pigment and is useful as the host strain for insertion site screening, called MWM1200 (U.S. Ser. No. 10/997,844; hereby incorporated by reference). This was accomplished through homologous recombination to inactivate the promoter driving expression of the crtN1aldcrtN2 gene cluster, and a separate homologous recombination to disrupt the crtN3 gene.

Two-step Homologous Recombination Process

As described in co-pending U.S. patent application Ser. No. 10/997,844, a high growth methanotrophic bacterial strain with no introduced selection marker and no endogenous carotenoid production was created using a designed 2-step homologous recombination process. This Methylomonas strain was used in the integration experiments that identified the tig region as a valuable target integration region.

The ability to produce specific defined mutations in a microorganism frequently relies on exploitation of the native homologous recombination properties of the cell to replace a nucleotide sequence of interest with a modified copy. Most frequently, the nucleotide sequence of interest is a particular functional gene of interest, which is then disrupted by the insertion of an antibiotic-resistance marker. In theory, this type of recombination event is easily detected on a selective medium; however, performing allelic exchange in C1 metabolizing microorganisms has been relatively cumbersome due to the organisms' slow growth rates and the rarity of double-crossover events (which require extensive screening to isolate an allelic-exchange mutant). Despite these difficulties, a positive selection method for the identification of allelic exchange mutants obtained by targeted homologous recombination has been developed, as described in co-pending U.S. patent application Ser. No. 10/997,308; corresponding to PCT/US04/40621 Briefly, the positive selection (or direct genetic selection) of mutant bacteria is possible whenever survival of the recombinant bacteria depends upon the presence or absence of a particular function encoded by the DNA that is introduced into the organism. The advantage of a selection method over a screening method is that growth of bacteria with the specific desired mutation is greatly favored over bacteria lacking that specific mutation, thus facilitating the identification of the preferred mutants.

Direct or positive selection vectors containing genes that convey lethality to the host are well known. For example, expression of the Bacillus subtilis or the B. amyloliquefaciens sacB genes in the presence of sucrose is lethal to E. coli and a variety of other Gram-negative and Gram-positive bacteria. The sacB gene encodes levansucrase, which catalyzes both the hydrolysis of sucrose and the polymerization of sucrose to form the lethal product levan. Although the basis for the lethality of levansucrase in the presence of sucrose is not fully understood, the inability of E. coli and many other gram negative bacteria to grow when sacB is expressed can be exploited to directly select for cells that have lost the sacB gene via homologous recombination. Numerous methods have been developed for the selection of various bacterial mutants, based on sacB. See for example: U.S. Pat. No. 6,048,694 (Bramucci et al.) concerning Bacillus; U.S. Pat. No. 5,843,664 (Pelicic et al.) concerning mycobacterium; U.S. Pat. No. 5,380,657 (Schaefer et al.) concerning Coryneform bacteria; Hoang et al. (Gene, 212(1):77-86 (1998)) concerning Pseudomonas aeruginosa; Copass et al. Infection and Immun., 65(5):1949-1952 (1997)) concerning Helicobacter pylori; and Kamoun et al. (Mol. Microbiol., 6(6):809-816 (1992)) concerning Xanthomonas.

The principle of the two-step positive selection strategy based on use of sacB for C1 metabolizing bacteria relies on the application of a positive selection vector which is able to integrate into the chromosome of C1 metabolizing bacteria to produce mutations that are the result of both single- or double-crossover events. Specifically, the positive selection vector comprises:
(i) at least one gene that functions as a first selectable marker (e.g., Amp, Kan resistance gene);
(ii) a sacB coding region encoding a levansucrase enzyme under the control of a suitable promoter; and
(iii) a replacement nucleotide sequence of interest (i.e., re-NSI), which one desires to insert into the chromosome of the C1 metabolizing bacteria as a replacement to an existing nucleotide sequence of interest in the bacterial chromosome (i.e., chr-NSI). Thus, re-NSI is modified with respect to chr-NSI by the addition, substitution, or deletion of at least one nucleotide.

Upon transformation of C1 metabolizing bacteria with the positive selection vector described above, a single-crossover event by homologous recombination occurs between chr-NSI and re-NSI, such that the entire positive selection vector is integrated into the bacterial chromosome at the site of crossover. These events can be selected by growth on the antibiotic corresponding to the first selectable marker (e.g., Amp or kan), whereby a complete copy of chr-NSI and a complete copy of re-NSI are present in the chromosome. Upon removal of selection for the first selectable marker, a second crossover event may occur, resulting in the “looping out” of the positive selection vector, to yield transformants containing either the chr-NSI or the re-NSI in the chromosome. Direct selection of these double-crossover transformants is possible by growing the transformants in the presence of sucrose, since single-crossover mutants still contain the sacB gene and will be killed under these conditions.

Screening Methods for Two-Step Selection

Methods of screening in microbiology are discussed at length in Brock, supra. In the present invention, a two-step selection process permits the identification of double-crossover events in C1 metabolizing bacterial cells by applying positive selection pressure. Using this strategy, the positive selection vector should comprise a first selectable marker and a sacB marker. Selection involves first growing the transformants on media containing the antibiotic corresponding to the first selectable marker, to identify those cells that have undergone a single-crossover (i.e., wherein the entire chromosomal integration vector has integrated into the host cell's genome). Then, the selection pressure is removed and a second crossover event may occur. Strains that undergo a double-crossover event can be obtained by a two step process. The first step is an enrichment process. The cells with a single-crossover are grown without selection pressure (grown in the absence of Kan, for example) and passaged at least several times by subculturing. The second step is the selection of strains that have lost the vector backbone containing the sacB gene. This selection process requires growth of the cells on sucrose, since SacB expression will be lethal to all single-crossover mutants. Differentiation between double-crossover lines containing the wildtype and mutant allele is then possible using standard molecular techniques (e.g., PCR), well known to one of skill in the art.

An advantage of the selection strategy described above is that double-crossover transformants that are produced no longer contain the selection marker derived from the vector.

Creation of Background Strain for Chromosomal Integration

The non-pigmented Methylomonas sp. 16a bacterial host organism MWM1200, lacking any antibiotic markers, and comprising deletions in the crtN1aldcrtN2 gene cluster promoter and the crtN3 gene, was used in experiments providing the invention herein. This bacterial strain was created by allelic exchange mutations within the native crtN1aldcrtN2 gene cluster promoter and the crtN3 gene of Methylomonas sp. 16a.

The process by which the allelic exchange mutations were created requires a re-NSI that is modified with respect to chr-NSI by the addition, substitution, or deletion of at least one nucleotide. For the purposes herein, the chr-NSI corresponds to a native crtN3 gene of Methylomonas sp. 16a or the promoter driving the Methylomonas sp. 16a crtN1aldcrtN2 gene cluster. And, the re-NSI will enable production of a transformant Methylomonas sp. 16a having a deletion in crtN3 or the promoter driving the crtN1aldcrtN2 gene cluster. The advantage of the two-step selection methodology described herein is that the allelic exchange mutant thus generated does not contain the selectable marker of the transforming plasmid; this enables subsequent mutations to be created using the same technique (i.e., since there is no need for a different selectable marker corresponding to each mutation created). Thus the crtN3 mutation was added to a crtN1aldcrtN2 gene cluster promoter mutant strain, combining the two mutations in one strain.

One factor to consider regardless of the specific type of re-NSI generated is the overall homology between the re-NSI and the chr-NSI. In general, it is well known in the art that homologous recombination generally requires a minimum of about 50 nucleotides of homology on each side of the site of a crossover. When preparing a re-NSI for use in the selection processes described herein, it is preferable to have regions homologous to the chr-NSI flanking (both 5′ and 3′) the site of the addition, substitution, or deletion. More preferably, a region of homology of about at least 1 kB is preferred on both sides of the addition, substitution, or deletion. In contrast, re-NSI is not expected to be limited in length, beyond the limitations inherent to homologous recombination.

Another factor to consider during the preparation of a re-NSI for use in the two-step selection strategy concerns the placement of the addition, deletion, or substitution within the sequence of interest. Specifically, the re-NSI is first inserted into the chromosome by integration of the chromosomal integration vector (a single-crossover event). The second crossover event that occurs can result in either a mutant or wildtype sequence in the chromosome, since the single-crossover contains two copies of the nucleotide sequence of interest. In order to increase the percentage of segregants that retain the re-NSI, as opposed to reverting to the wildtype encoded by the chr-NSI, it is desirable to “center” the mutation with respect to the flanking DNA that has homology to the chr-NSI. For example, if a point mutation was perfectly centered within a re-NSI, about 50% of the segregants would be expected to retain the mutation in the chromosome (thus producing a 1:1 ratio of allelic exchange mutants to wild-type cells. Transformation of C1 Metabolizing Bacteria

Electroporation has been used successfully for the transformation of: Methylobacterium extorquens AM1 (Toyama, H., et al., FEMS Microbiol. Lett., 166:1-7 (1998)), Methylophilus methylotrophus AS1 (Kim, C. S., and T. K. Wood. Appl. Microbiol. Biotechnol., 48: 105-108 (1997)), and Methylobacillus sp. strain 12S (Yoshida, T., et al., Biotechnol. Lett., 23: 787-791 (2001)). Extrapolation of specific electroporation parameters from one specific C1 metabolizing utilizing organism to another may be difficult, however, as is well to known to those of skill in the art.

Bacterial conjugation, relying on the direct contact of donor and recipient cells, is frequently more readily amenable for the transfer of genes into C1 metabolizing bacteria. Simplistically, this bacterial conjugation process involves mixing together “donor” and “recipient” cells in close contact with one another. Conjugation occurs by formation of cytoplasmic connections between donor and recipient bacteria, with direct transfer of newly synthesized donor DNA into the recipient cells. As is well known in the art, the recipient in a conjugation is defined as any cell that can accept DNA through horizontal transfer from a donor bacterium. The donor in conjugative transfer is a bacterium that contains a conjugative plasmid, conjugative transposon, or mobilizable plasmid. Although the detailed mechanism of transfer is not that well understood, the physical transfer of the donor plasmid can occur in one of two fashions, as described below:
1. In some cases, only a donor and recipient are required for conjugation. This occurs when the plasmid to be transferred is a self-transmissible plasmid that is both conjugative and mobilizable (i.e., carrying both tra genes and genes encoding the Mob proteins). In general, the process involves the following steps: 1.) Double-strand plasmid DNA is nicked at a specific site in oriT; 2.) A single-strand DNA is released to the recipient through a pore or pilus structure; 3.) A DNA relaxase enzyme cleaves the double-strand DNA at oriT and binds to a release 5′ end (forming a relaxosome as the intermediate structure); and 4.) Subsequently, a complex of auxiliary proteins assemble at oriT to facilitate the process of DNA transfer.
2. Alternatively, a “triparental” conjugation is required for transfer of the donor plasmid to the recipient. In this type of conjugation, donor cells, recipient cells, and a “helper” plasmid participate. The donor cells carry a mobilizable plasmid or conjugative transposon. Mobilizable vectors contain an oriT, a gene encoding a nickase, and have genes encoding the Mob proteins; however, the Mob proteins alone are not sufficient to achieve the transfer of the genome. Thus, mobilizable plasmids are not able to promote their own transfer unless an appropriate conjugation system is provided by a helper plasmid (located within the donor or within a “helper” cell). The conjugative plasmid is needed for the formation of the mating pair and DNA transfer, since the plasmid encodes proteins for transfer (Tra) that are involved in the formation of the pore or pilus.

Examples of successful conjugations involving C1 metabolizing bacteria include the work of: Stolyar et al. (Mikrobiologiya 64(5): 686-691 (1995)); Motoyama, H. et al. (Appl. Micro. Biotech. 42(1): 67-72 (1994)); Lloyd, J. S. et al. (Archives of Microbiology 171(6): 364-370 (1999)); and Odom, J. M. et al. (WO 02/18617; corresponding to U.S. Ser. No. 09/941,947).

Creation of Random Integration Library Using Single-Crossover Process

As described above, integration occurs based on the homology between the re-NSI DNA sequence in the introduced integration vector and the chr-NSI DNA sequence in the host cell genome. Instead of a re-NSI sequence in the vector, a DNA fragment of the target genome, called a homology region, may be cloned into the integration vector. The h-NSI (homology-NSI) sequence has not been modified so it is fully homologous to the chr-NSI sequence. In the first single-crossover step described above (the second sacB marker is not needed), the entire plasmid carrying the h-NSI is integrated into the genome at the location of the same DNA sequence in the chromosome. Genes of interest for genetic engineering of a host strain can be integrated into the host chromosome as part of the integration vector in this manner. The integrated genes will therefore be integrated in a chromosomal location that is adjacent to the DNA sequence that comprises the chr-NSI. If random genomic DNA fragments are cloned into integration vectors and are used as homology regions, then integration will occur for each vector in the location of the specific genomic DNA fragment in that vector. Random genomic fragments can be prepared, for example, by shearing genomic DNA or by digestion with a restriction enzyme that recognizes a four base sequence and so cuts the DNA at many locations, such as Sau3A. These random DNA fragments from Methylomonas genome were cloned upstream of the genes for integration. Genes carried on the integration vector with h-DNA will be integrated adjacent to the chromosomal homology region sequence. This process provides a means of randomly integrating genes into the chromosomal DNA of a host organism. Since the entire vector is integrated in this step, the selection marker on the vector will be present in the genome as well.

As described above for the re-NSI, one factor to consider regardless of the specific h-NSI is the overall homology between the h-NSI and the chr-NSI. In general, it is well known in the art that homologous recombination requires a minimum of about 50 nucleotides of homology on each side of the site of a crossover. When preparing an h-NSI for use in the selection processes described herein, it is preferable to have at least about a 1 kB region of homology to the chr-NSI. More preferably, at least about a 1 to 2 kB region of homology is preferred. In contrast, h-NSI is not expected to be limited in length, beyond the limitations inherent to homologous recombination.

Identification of Stable High Expression Integration Region

Through the process described above, any gene or nucleic acid molecule can be integrated at random sites in the genome. Visual screening for products of expression of a screening marker gene can be used to assay thousands of individual integration events in parallel to possibly identify a rare high expression site. In experiments leading to the current invention, a DNA fragment comprising the promoterless crtEWYIB gene cluster was randomly integrated in the non-pigmented Methylomonas sp. 16a strain MWM1200 genome using Sau3A h-DNA fragments. These genes function as a reporter gene set for production of the orange pigment, canthaxanthin. Through the screening of thousands of lines, any rare events with high expression of the integrated reporter genes can be identified, such as lines with a bright orange color. If lines with high expression are identified, the genomic DNA from these lines can then be characterized to identify the integration site of the reporter gene(s) through sequencing the DNA surrounding the integrated reporter gene(s). Further analysis of the surrounding DNA sequences using sequence analysis software such as the GCG suite of programs ((Wisconsin Package Version 9.0, Genetics Computer Group (GCG), Madison, Wis.); BLASTP, BLASTN, BLASTX (Altschul et al., J. Mol. Biol., 215:403-410 (1990)); DNASTAR (DNASTAR, Inc., Madison, Wis.); and the FASTA program incorporating the Smith-Waterman algorithm (W. R. Pearson, Comput. Methods Genome Res., [Proc. Int. Symp.], Meeting Date 1992,111-20. Suhai, Sandor, Ed.; Plenum: New York, N.Y. (1994)) locates ORFs (including orientation) and determines the identities of those ORFs through DNA or protein homology to known sequences. A map of ORFs and putative promoter regions may be constructed based on the results of the sequence analysis. The map allows the determination of how the integrated gene is being expressed: what promoter is used, and whether it is part of an operon.

Through this random integration, screening, and characterization process, the tig region of the invention was identified as a genomic location that confers a stable and high level expression of the integrated genes.

Composition of the tig Region

High expression was found when the crtEWYIB genes were integrated in an ORF that is predicted to encode a protein with high amino acid similarity to the Lon protease. The Lon protease gene has been identified in E. coli and other bacteria such as Myxococcus xanthus, Bacillus brevis and Erwinia amylovora. Strains that have mutations in the Ion gene have increased uv sensitivity and elevated levels of extracellular polysaccharide, but the Ion gene is not essential for growth. High expression of the E. coli alpA gene (Alternative Lon protease) suppresses the Ion mutant phenotypes. Thus lines of Methylomonas sp. 16a MWM1200 with interruption of Lon protease expression due to insertion of the crtEWYIB genes were viable and showed high expression based on the presence of high levels of canthxanthin observed by the intense orange color.

Further sequence analysis of the region surrounding the ion gene showed that this Ion gene is one ORF in a gene cluster that includes six ORFs with the same orientation that all appear to encode proteins that are involved in protein metabolism (FIG. 2). The first ORF of this cluster (SEQ ID NO:11) encodes a protein (SEQ ID NO:12) with sequence similarity to trigger factor, Tig. The second ORF in the cluster (SEQ ID NO:13) encodes a protein (SEQ ID NO:14) with similarity to ClpP, the third ORF (SEQ ID NO:15) encodes a protein (SEQ ID NO:16) with sequence similarity to ClpX, the fourth ORF (SEQ ID NO:17) encodes a protein (SEQ ID NO:18) with sequence similarity to Lon protease, the fifth ORF (SEQ ID NO:19) encodes a protein (SEQ ID NO:20) with sequence similarity to HimA, and the sixth ORF (SEQ ID NO:21) encodes a protein (SEQ ID NO:22) with sequence similarity to ppiC. This cluster of genes is structured such that each gene does not have its own promoter, but a promoter for expression of the entire cluster lies upstream of the tig gene. This tig promoter directs the transcription of the entire tig-cipP-cipX-Ion-himA-ppiC gene region. The sequence of the entire tig region identified in Methylomonas sp. 16a is given as SEQ ID NO:1.

The protein encoded by the first gene of the cluster, Trigger factor, has been found in E. coli to be bound to ribosomes in a stoichiometry of approximately 1:1, and the protein's role is to bind nascent amino acid sequences to aid in proper folding of newly synthesized proteins (Hesterkakamp et al., PNAS, 93:437-4441 (1996)). Though the tig gene is known to be highly expressed in some other organisms, it is unknown as to whether it would also have high expression in specialized organisms such as methylotrophs and/or methanotrophs. In addition, in hosts where it is known to be highly expressed, it is only one of many highly expressed bacterial genes. Furthermore, it is unknown whether integration of a large cluster in this region will be stable. As a result, this finding could not have been predicted.

Integration Within the tig Region

Though the insertions identified in the screen were located within the Lon protease coding region, it is preferable to insert gene(s) in a location such that expression of a host coding region is not disrupted, while retaining expression. One skilled in the art will know that a gene that is integrated within this tig region downstream of, or 3′ to, the tig promoter will be transcribed along with the other genes in the cluster. Thus, for expression, an integrated gene must be 3′ to the promoter for the tig region. The coding regions of the genes in the cluster are expressed from the same initial transcript. All of the coding regions in the tig region gene cluster are oriented with the same 5′ to 3′ polarity. An introduced gene must be integrated such that the orientation of the coding region is the same as the orientation of the other coding regions in the tig region gene cluster.

A gene may be integrated in the tig region in any location that allows its expression and does not compromise the host strain. It is obvious to one skilled in the art that integration within a coding region of the tig region gene cluster would affect expression of the encoded protein. This in turn may affect the normal cell functions. The protein encoded by an introduced gene would not be expressed if it were inserted within another coding region, but out of frame. The protein encoded by an introduced gene also may not be expressed if it is inserted within a coding region such that it is translated as a fusion protein that cannot function normally. Therefore it is desirable to integrate a gene in non-coding sequence that lies between two coding regions in the tig region to avoid disruption of the expression of any encoded proteins and to ensure function of the expressed introduced gene product. One skilled in the art will know how to target the integration of an introduced gene, by using as the h-NSI a DNA fragment with sequence that is adjacent to the desired integration site in the case of single-crossover integration. If a double-crossover event is desired, the h-NSI includes two DNA segments, derived from sequences on either side of the target integration site. The gene to be integrated is cloned in the integration vector between these two DNA segments. Either single or double-crossover integration may be used. Double-crossover integration has the advantage of elimination of vector sequences that include a selection marker.

A single gene, or multiple genes may be integrated together in one location in the tig region. Alternatively, two or more genes may be integrated separately at different locations in the tig region. Integration of one gene between the tig and clpP genes, and integration of a second gene between the clpX and Ion genes of the Methylomonas sp. 16a tig region is an example of multiple, separate gene integration. Preferred integration of the invention is double-crossover integration targeted to the non-coding region between two coding regions of the tig region gene cluster. Most preferred is double-crossover integration between the tig and clpP genes.

Integration Vector for tig Region

Any integration vector may be used for integration of a gene(s) into the tig region, providing that the vector contains a DNA seqment that is homologous to a portion of the genomic tig region. As described above, this h-NSI may be as short as about 0.5 kB in length, is preferably of at least about 1 kB in length and more preferred is at least about 1 to 2.4 kB in length. The genomic tig region sequence is expected to have variations when derived from different methylotrophs, or different methanotrophs, or even different strains of Methylomonas. The exact DNA sequence of the tig region is not a requirement for the invention. One skilled in the art will recognize a tig region based on DNA sequence similarity and % similarity of the encoded proteins, as compared to the tig region identified herein from Methylomonas sp. 16a. Any methylotroph with a tig region may be used to practice the invention. One skilled in the art will know that the h-NSI DNA fragment used in an integration vector is homologous to the chr-NSI sequence of the integration host strain. The method of integration is not critical, and can be for example by single-crossover, or double-crossover.

Identification of a tig Region

The genomic tig region is expected to have variations in both sequence and structure when derived from different methylotrophs, or different methanotrophs, or even different strains of Methylomonas. The exact DNA sequence of the tig region is not a requirement for the invention. Any methylotroph with a tig region may be used to practice the invention. Examples of methylotrophs that may be used to practice the invention include, but are not limited to: Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, and Pseudomonas.

The tig region provides a target location for chromosomal integration of genes in other microorganisms, including E. coli and other gram-negative and positive bacteria.

A tig region comprises the ORFs downstream of and adjacent to the tig promoter that do not have additional promoters but are transcribed using the tig promoter. The tig region may include the tig-clpP-clpX-Ion-himA-ppiC gene cluster as in Methylomonas sp. 16a, or may have substitutions of other genes. The tig region may have a reduced, or a larger number of genes. The tig region must have a coding region for Trigger factor as the 5′ most ORF, and in the minimal situation, the Trigger factor coding region is the only ORF whose transcription is directed by the tig promoter.

One skilled in the art will recognize a tig region based on DNA sequence similarity and amino acid similarity of the encoded protein(s). The sequence of the Trigger protein (SEQ ID NO:12) is a preferred identifier for the tig region. A tig region may be identified through sequence analysis of genomic DNA sequences using sequence analysis software, or may be cloned using a probe made from the Methylomonas sp. 16a tig region, preferably from the tig gene.

Genes for Integration in the tig Region

Metabolic engineering generally requires the introduction of a gene or genes whose expression leads to altered metabolism. It is usually desired that the introduced gene havehigh expression. In cases where a product is to be produced through large scale growth in a bioreactor, the lack of a selection marker, stability of the introduced gene, and normal growth rate of the host microorganism are also important. Thus for many metabolic engineering projects, integration in the tig region may provide the desired properties. Any gene that is useful for metabolic engineering may be integrated in the tig region. Additionally genes encoding proteins that in themselves are of commercial value may be expressed in the tig region integration system. The genes for integration may be either endogenous to the host or heterologous and must be compatible with the host organism. For example, suitable genes of interest may include, but are not limited to those encoding viral, bacterial, fungal, plant, insect, or vertebrate proteins of interest, including mammalian polypeptides. Further, these genes of interest may be, for example, structural proteins, enzymes, or peptides. As will be obvious to one skilled in the art, the particular functionalities required to be introduced into a host organism for production of a particular product will depend on the host cell, the availability of substrate, and the desired end product(s).

A particularly preferred, but non-limiting list includes:
1) genes encoding enzymes involved in the central carbon pathway, such as transaldolase, fructose bisphosphate aldolase, keto deoxy phosphogluconate aldolase, phosphoglucomutase, glucose-6-phosphate isomerase, phosphofructokinase, 6-phosphogluconate dehydratase, 6-phosphogluconate-6-phosphate-1 dehydrogenase, and the like;
2) genes encoding enzymes involved in the production of isoprenoid molecules, such as 1-deoxyxylulose-5-phosphate synthase (dxs), 1-deoxyxylulose-5-phosphate reductoisomerase (dxr), geranyltransferase or farnesyl diphosphate synthase (ispA), 2C-methyl-D-erythritol cytidyltransferase (ispD), to 4-diphosphocytidyl-2-C-methylerythritol kinase (ispE), 2C-methyl-d-erythritol 2,4-cyclodiphosphate synthase (ispF), and geranylgeranyl pyrophosphate synthase (crtE);
3) genes encoding carotenoid pathway enzymes such as zeaxanthin glucosyl transferase (crtX), lycopene cyclase (crtY), phytoene dehydrogenase (crtI), phytoene synthase (crtB), beta-carotene hydroxylase (crtZ), phytoene desaturase (crtD), beta-carotene ketolase (crtO, crtW), and the like, which would enable the production of carotenoids such as antheraxanthin, astaxanthin, canthaxanthin, alpha-carotene, beta-carotene, epsilon-carotene, gamma-carotene, ζ-carotene, alpha-cryptoxanthin, diatoxanthin, 7,8-didehydroastaxanthin, fucoxanthin, fucoxanthinol, lactucaxanthin, lutein, lycopene, neoxanthin, neurosporene, peridinin, phytoene, rhodopin, rhodopin glucoside, siphonaxanthin, spheroidene, spheroidenone, spirilloxanthin, uriolide, uriolide acetate, violaxanthin, and zeaxanthin;
4) genes encoding cyclic terpenoid synthases (e.g., limonene synthase) for the production of terpenoids, and the like;
5) genes encoding enzymes involved in the production of exopolysaccharides, such as UDP-glucose pyrophosphorylase (ugp), glycosyltransferase (gumD), polysaccharide export proteins (wza, espB), polysaccharide biosynthesis (espM), glycosyltransferase (waaE), sugar transferase (espV), galactosyltransferase (gumH), and glycosyltransferase genes and the like;
6) genes encoding enzymes involved in the production of aromatic amino acids, such as 3-deoxy-D-arabinoheptulosonate-7-phosphate synthase (aroG), 3-dehydroquinate synthase (aroB), 3-dehydroquinase or 3 dehydroquinate dehydratase (aroQ), 5-shikimic acid dehydrogenase (aroE), shikimic acid kinase (aroK), 5-enolpyruvylshikimate-3-phosphate synthase, chorismate synthase (aroC), anthranilate synthase (trpE), anthranilate phosphoribosyltransferase (trpD), indole 3-glycerol phosphate synthase (trpC), tryptophan synthetase (trpB), chorismate mutase or prephenate dehydratase (pheA), and prephenate dehydrogenase (tyrAc); and
7) pds, phaC, phaE, efe, pdc, and adh genes and genes encoding pinene synthase, bornyl synthase, phellandrene synthase, cineole synthase, sabinene synthase, and taxadiene synthase, respectively.

The preferred genes of 3) above include, but are not limited to crtE, crtB, crtI, crtY, crtZ and crtX genes isolated from Pectobacterium cypripedii, as described in U.S. patent application Ser. No. 10/804,677, incorporated herein by reference; crtE, crtB, crtI, crtY, crtZ and crtX genes isolated from a member of the Enterobacteriaceae family, as described in U.S. patent application Ser. No. 10/808,979, incorporated herein by reference; crtE, idi, crtB, crtI, crtY, crtZ genes isolated from Pantoea agglomerans, as described in U.S. patent application Ser. No. 10/808,807, incorporated herein by reference; and crtE, idi, crtB, crtI, crtY, crtZ and crtX genes isolated from Pantoea stewartii, as described in U.S. patent application Ser. No. 10/810,733, incorporated herein by reference. More preferably, the crtE-idi-crtY-crtI-crtB gene cluster, given as SEQ ID NO:6, derived from the crtE-idi-crtY-crtI-crtB-crtZ gene cluster (SEQ ID NO:41) isolated from Pantoea agglomerans, described in U.S. patent application Ser. No. 10/808,807, is used.

For coding regions with codon usage that is not optimal for expression in the host bacterium, it is desirable to modify a portion of the codons to enhance the expression the encoded polypeptides in a methylotroph, or specifically in Methylomonas sp. 16a and derivatives thereof. Thus, the nucleic acid sequence of the native β-carotene ketolase gene from Agrobacterium aurantiacum, was modified to employ host preferred codons, as described in Example 8 (U.S. Ser. No. 10/997,844). In general, host preferred codons can be determined from the codons of highest frequency in the proteins (preferably expressed in the largest amount) in a particular host species of interest. Thus, the coding sequence for a polypeptide having ketolase activity can be synthesized in whole or in part using the codons preferred in the host species. All (or portions) of the DNA also can be synthesized to remove any destabilizing sequences or regions of secondary structure which would be present in the transcribed mRNA. All (or portions) of the DNA also can be synthesized to alter the base composition to one more preferable in the desired host cell.

In one preferred embodiment, the crtE-idi-crtY-crtI-crtB gene cluster (SEQ ID NO:6) from Pantoea agglomerans is used in conjunction with the codon-optimized crtW (β-carotene ketolase) gene given as SEQ ID NO:7 to produce the C₄₀ carotenoid canthaxanthin.

Applications for the Integration Site Expression System.

As is well known to those of skill in the art, efforts to genetically engineer a microorganism for high-level production of a specific product frequently require high level expression of one or more introduced genes. For large-scale production the introduced gene(s) must be stably maintained, and preferably without the requirement for an antibiotic or nutritional selection. The present invention represents tremendous progress in the genetic engineering of methylotrophic bacteria. Specifically, the integration site within the tig region provides for a relatively high expression and stable system for introduced gene(s), such as those described above, with no requirement for a selection marker. Growth of the host strain harboring the tig region integration also remains at a level only slightly reduced from that of the non-integration host.

Preferred is use of the tig region integration system for expression of genes encoding enzymes involved in carotenoid synthesis in Methylomonas sp. 16a providing a new platform for production of carotenoids. More preferred is use of this system for expression of genes for C₄₀ carotenoid synthesis in Methylomonas sp. 16a MWM1200 (non-pigmented mutant strain) providing a platform for production of C₄₀ carotenoids. For example, products include, but are not limited to C₄₀ carotenoids, such as antheraxanthin, adonirubin, adonixanthin, astaxanthin, canthaxanthin, capsorubrin, β-cryptoxanthin, α-carotene, β-carotene, epsilon-carotene, echinenone, 3-hydroxyechinenone, 3′-hydroxyechinenone, Γ-carotene, 4-keto-Γ-carotene, ζ-carotene, α-cryptoxanthin, deoxyflexixanthin, diatoxanthin, 7,8-didehydroastaxanthin, fucoxanthin, fucoxanthinol, isorenieratene, lactucaxanthin, lutein, lycopene, myxobactone, neoxanthin, neurosporene, hydroxyneurosporene, peridinin, phytoene, rhodopin, rhodopin glucoside, 4-keto-rubixanthin, siphonaxanthin, spheroidene, spheroidenone, spirilloxanthin, 4-keto-torulene, 3-hydroxy-4-keto-torulene, uriolide, uriolide acetate, violaxanthin, zeaxanthin-β-diglucoside, and zeaxanthin.

Stability Requirement for Commercial Production

For commercial fermentation production of a product using an engineered microbial host, stability of the introduced genes required for product synthesis must last for over a hundred generations. Chromosomal integration in the tig region of the invention provides this level of stability. Integration through homologous recombination as practiced in the present invention, as compared to integration through transposon mutagenesis, has the advantage that once inserted into the chromosome the likelihood of further movement within the genome is very small. Chromosomal insertion provides the most segregationally stable expression system for foreign DNA since the foreign DNA is passed on to progeny as a part of normal chromosomal replication and since, theoretically, the foreign DNA can only be lost as a result of a recombination event.

Industrial Production Methodologies

Where expression of a suitable coding region of interest is desired using the tig region integration system of the instant invention for commercial production of a product, a variety of culture methodologies may be applied. For example, large-scale production of a specific product made possible by integrated gene expression in a recombinant microbial host may be accomplished by both batch and continuous culture methodologies.

A classical batch culturing method is a closed system where the composition of the media is set at the beginning of the culture and not subject to external alterations during the culturing process. Thus, at the beginning of the culturing process the media is inoculated with the desired organism or organisms and growth or metabolic activity is permitted to occur while adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, 2nd ed. (1989) Sinauer Associates: Sunderland, Mass., or Deshpande, Mukund V., Appl. Biochem. Biotechnol., 36:227 (1992).

Commercial production of a product of interest in a C1 metabolizing bacteria may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively continuous culture may be practiced with immobilized cells where carbon and nutrients are continuously added, and valuable products, by-products and waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes, as well as techniques for maximizing the rate of product formation, are well known in the art of industrial microbiology and a variety of methods are detailed by Brock, supra.

Fermentation media in the present invention must contain suitable carbon substrates. Suitable carbon substrates for the optimized Methylomonas sp. 16a host cells of the present invention include methane and methanol for which metabolic conversion into key biochemical intermediates has been demonstrated.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### GENERAL METHODS

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory: Cold Spring Harbor, N.Y. (1989) (Maniatis); by T. J. Silhavy, M. L. Bennan, and L. W. Enquist, Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987). The meaning of abbreviations is as follows: “sec” means second(s), “min” means minute(s), “hr” means hour(s), “d” means day(s), “μL” means microliter(s), “mL” means milliliter(s), “L” means liter(s), “μM” means micromolar, “mM” means millimolar, “M” means molar, “mmol” means millimole(s), “μmol” mean micromole(s), “nmol” means nanomole(s), “g” means gram(s), “μg” means microgram(s), “ng” means nanogram(s), “nm” means nanometers, “U” means unit(s), “ppm” means parts per million, “bp” means base pair(s), “rpm” means revolutions per minute, “kB” means kilobase(s), “g” means the gravitation constant, “OD₆₀₀” means the optical density measured at 600 nm, “OD₂₆₀/OD₂₈₀” means the ratio of the optical density measured at 260 nm to the optical density measured at 280 nm, “psig” means pounds per square inch guage, and “mAU” means milliabsorbance units.

Molecular Biology Techniques:

Methods for agarose gel electrophoresis were performed as described in Maniatis (supra). Polymerase Chain Reactions (PCR) techniques were found in White, B., PCR Protocols: Current Methods and Applications, Humana: Totowa, N.J. (1993), Vol. 15.

Media and Culture Conditions:

General materials and methods suitable for the maintenance and growth of bacterial cultures are found in: Experiments in Molecular Genetics (Jeffrey H. Miller), Cold Spring Harbor Laboratory: Cold Spring Harbor, N.Y. (1972); Manual of Methods for General Bacteriology (Phillip Gerhardt, R. G. E. Murray, Ralph N. Costilow, Eugene W. Nester, Willis A. Wood, Noel R. Krieg and G. Briggs Phillips, eds.), American Society for Microbiology: Washington, D.C., pp 210-213; or, Thomas D. Brock in Biotechnology: A Textbook of Industrial Microbiology, 2^{nd} ed. Sinauer Associates: Sunderland, Mass. (1989). All reagents and materials used for the growth and maintenance of bacterial cells were obtained from Aldrich Chemicals (Milwaukee, Wis.), BD Diagnostic Systems (Sparks, Md.), Invitrogen Corp. (Carlsbad, Calif.), or Sigma Chemical Company (St. Louis, Mo.), unless otherwise specified.

### Example 1

### Growth of Methylomonas Sp. 16a

Example 1 summarizes the standard conditions used for growth of Methylomonas sp. 16a (ATCC# PTA-2402), as described in WO 02/20728, correspoding to U.S. Pat. No. 6,689,601, hereby incorporated by reference.

Methylomonas Strain and Culture Media

The growth conditions described below were used throughout the following experimental Examples for treatment of Methylomonas 16a, unless conditions were specifically described otherwise.

Methylomonas sp. 16a was typically grown in serum stoppered Wheaton bottles (Wheaton Scientific; Wheaton, Ill.) using a gas/liquid ratio of at least 8:1 (i.e., 20 mL or less of ammonium liquid “BTZ” growth medium in a Wheaton bottle of 160 mL total volume). The composition of the BTZ growth medium is given below. The standard gas phase for cultivation contained 25% methane in air, although methane concentrations can vary ranging from about 5-50% by volume of the culture headspace. These conditions comprise growth conditions and the cells are referred to as growing cells. In all cases, the cultures were grown at 30° C. with constant shaking in a rotary shaker (Lab-Line, Barnstead/Thermolyne; Dubuque, Iowa) unless otherwise specified.

BTZ Media for Methylomonas 16a

Methylomonas 16a typically grows in a defined medium composed of only minimal salts; no organic additions such as yeast extract or vitamins are required to achieve growth. This defined medium known as BTZ medium (also referred to herein as “ammonium liquid medium”) consisted of various salts mixed with Solution 1, as indicated in Tables 1 and 2. Alternatively, the ammonium chloride was replaced with 10 mM sodium nitrate to give “BTZ (nitrate) medium”, where specified. Solution 1 provides the composition for a 100-fold concentrated stock solution of trace minerals.

TABLE 1
Solution 1*

| | Molecular | Conc. | |
|---|---|---|---|
| | Weight | (mM) | g per L |
| Nitriloacetic acid | 191.10 | 66.90 | 12.80 |
| CuCl₂ × 2H₂O | 170.48 | 0.15 | 0.0254 |
| FeCl₂ × 4H₂O | 198.81 | 1.50 | 0.30 |
| MnCl₂ × 4H₂O | 197.91 | 0.50 | 0.10 |
| CoCl₂ × 6H₂O | 237.90 | 1.31 | 0.312 |
| ZnCl₂ | 136.29 | 0.73 | 0.10 |
| H₃BO₃ | 61.83 | 0.16 | 0.01 |
| Na₂MoO₄ × 2H₂O | 241.95 | 0.04 | 0.01 |
| NiCl₂ × 6H₂O | 237.70 | 0.77 | 0.184 |

*Mix the gram amounts designated above in 900 mL of H₂O, adjust to pH = 7.0, and add H₂O to a final volume of 1 L. Keep refrigerated.

TABLE 2
Ammonium Liquid Medium (BTZ)**

| | | Conc. | | |
|---|---|---|---|---|
| | MW | (mM) | g per L | |
| NH₄Cl | 53.49 | 10 | 0.537 | |
| KH₂PO₄ | 136.09 | 3.67 | 0.5 | |
| Na₂SO₄ | 142.04 | 3.52 | 0.5 | |
| MgCl₂ × 6H₂O | 203.3 | 0.98 | 0.2 | |
| CaCl₂ × 2H₂O | 147.02 | 0.68 | 0.1 | |
| 1 M HEPES (pH 7.0) | 238.3 | | 50 | mL |
| Solution 1 | | | 10 | mL |

**Dissolve in 900 mL H₂O. Adjust to pH = 7.0, and add H₂O to give a final volume of 1 L. For agar plates: Add 15 g of agarose in 1 L of medium, autoclave, cool liquid solution to 50° C., mix, and pour plates.

Plates were incubated in a closed jar with 25% methane at 30° C.

### Example 2

### Construction of a Positive-Selection Suicide Vector for Methylomonas sp. Strain 16a

The construction of chromosomal mutations within the Methylomonas genome required the use of suicide vectors. Thus, a modified version of the conditional replication vector pGP704 was created, comprising an npr-sacB cassette.

pGP704 as a Vector Backbone for the C₁-Chromosomal Integration Vector

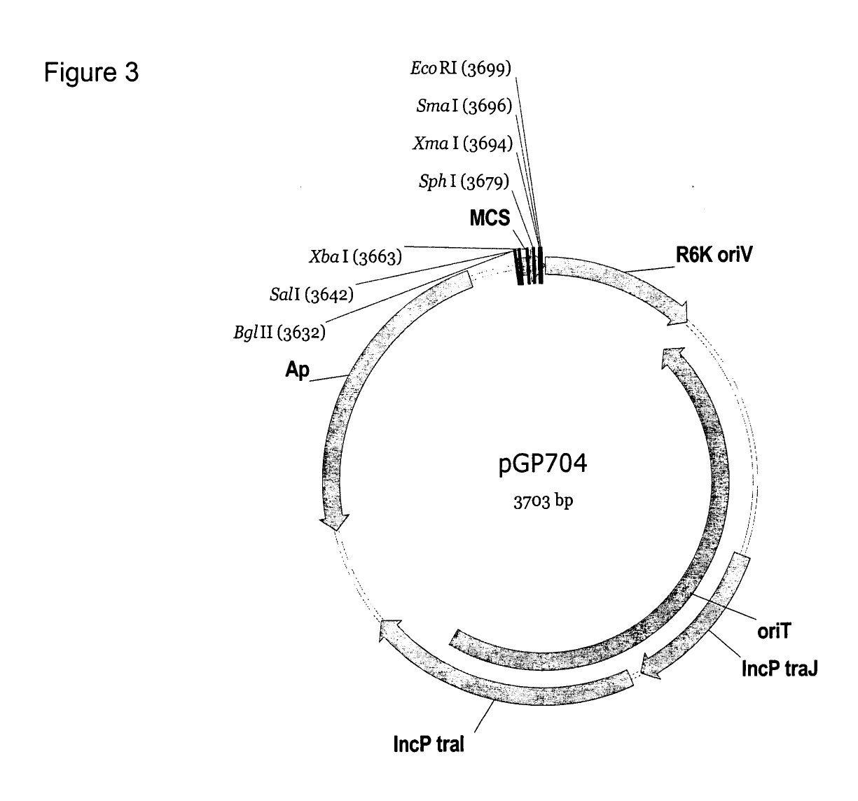

The plasmid pGP704 (Miller and Mekalanos, J. Bacteriol., (170): 2575-2583 (1988); FIG. 3) was chosen as a suitable vector backbone for the C1 chromosomal integration vector, since it could be used as a vehicle to transfer replacement nucleotide sequences of interest into Methylomonas sp. 16a via conjugation. Plasmid pGP704 is a derivative of pBR322 that is Amp^{R} but has a deletion of the pBR322 origin of replication (oriE1). Instead, the plasmid contains a cloned fragment containing the origin of replication of plasmid R6K. The R6K origin of replication (oriR6K) requires the π protein, encoded by the pir gene. In E. coli, the n protein can be supplied in trans by a prophage (λ pir) that carries a cloned copy of the pir gene. The pGP704 plasmid also contains a 1.9 kB BamHI fragment encoding the mob region of RP4. Thus, pGP704 can be mobilized into recipient strains by transfer functions provided by a derivative of RP4 integrated in the chromosome of E. coli strain SM10 or SY327. Once the plasmid is transferred, however, it is unable to replicate in recipients that lack the π protein (e.g., recipients such as Methylomonas and other C1 metabolizing bacteria). This inability permits homologous recombination to occur between nucleotide sequences of interest on pGP704 and the intact chromosomal nucleotide sequences of interest.

Thus, on the basis of the above characteristics, the pGP704 vector backbone met the following conditions for a chromosomal integration vector suitable for C1 metabolizing bacteria: 1.) it was conditional for replication, thus allowing selection for integration into the chromosome; 2.) it possessed at least one selectable marker; 3.) it had an origin of transfer that was expected to be suitable for C1 metabolizing bacteria; 4.) it possessed mobilization genes; and 5.) it contained a variety of unique cloning sites. Other alternative chromosomal integration vectors having the characteristics listed above are expected to be suitable for use in the present invention, as described herein.

pGP704 did not, however, permit easy detection and identification of clones that had undergone allelic exchange. Thus, pGP704 was modified to permit the positive selection of double-crossover events within Methylomonas and other C1 metabolizing bacteria.

Cloning of the npr-sacB Cassette

Plasmid pBE83 contained a Bacillus amyloliquifaciens sacB gene under the control of the neutral protease (npr) promoter (gift from V. Nagarajan, E.I. du Pont de Nemours and Co., Inc., Wilmington, Del.). The npr-sacB cassette was PCR amplified from pBE83 using DNA primers DrdI/npr-sacB and TthIII/npr-sacB. The DNA primers were constructed to include unique restriction sites at each terminus of the PCR product to facilitate subsequent cloning (as indicated by the underlined sequences below):

SEQ ID NO:9:

5′-GACATCGATGTCGAATTCGAGCTCGGTACCGATC-3′

SEQ ID NO:10:

5′-GACCTCGTCGCTGTTATTAGTTGACTGTCAGC-3′

The PCR reaction mixture was composed of the following: 10 μL of 10×PCR buffer; 16 μL (4 μL each) of dNTPs (320 mM stock); 1 μL of Methylomonas chromosomal DNA solution (˜500 ng/μL); 8 μL of MgCl₂ solution (25 mM); 0.5 μL of Taq polymerase (5 U/μL); 1 μL of DrdI/npr-sacB primer (˜36 nmol); 1 μL of TthIII/npr-sacB primer (−35 nmol); and 71 μL of sterile deionized water (NANOpure® Water System, Barnstead/Thermolyne). The PCR protocol was then performed on a 9600 GeneAmp® PCR System (Perkin Elmer), according to the thermocycling parameters below:
1 cycle: 94° C. (5 min);
1 cycle: 94° C. (5 min), 60° C. (2 min), 72° C. (3 min);
35 cycles: 94° C. (1 min), 60° C. (2 min), 72° C. (3 min);
1 cycle: 94° C. (1 min), 60° C. (2 min), 72° C. (10 min); and
Hold −4° C. (∞).
   Afterward, the PCR product was ligated into the pCR2.1TOPO vector per the manufacturer's instructions (Invitrogen; Carlsbad, Calif.). The ligation mixture was transformed into TOP10 One Shot™ calcium chloride competent cells and transformants were screened as recommended by Invitrogen. Plasmid DNA was isolated from positive clones (white colonies in a blue/white screen) using the QIAprep® Spin Mini-prep Kit (Qiagen; Valencia, Calif.) and the DNA was digested according to the manufacturer's instructions with restriction endonucleases DrdI and TthIII (New England Biolabs; Boston, Mass.). Initially, this PCR product was to be inserted into pGP704 digested with DrdI and TthIII; however, there were difficulties in cloning the DrdI/TthIII PCR product.

A modified cloning strategy was adopted, such that the PCR reaction described above was “repeated” using the Pfu DNA polymerase (Stratagene; LaJolla, Calif.). Specifically, the PCR reaction and protocol were performed exactly as described above, with the exception that Pfu polymerase and buffers from Stratagene were used. A PCR product having flush or blunt ends was produced. This PCR product was ligated directly into the Xcal site of pGP704. The ligation mixture was transformed into calcium chloride competent E. coli SY327 cells (Miller, V. L. and Mekalanos, J. J., Proc. Natl. Acad. Sci., 81(11):3471-3475 (1984)). The transformants were screened using the DrdI/npr-sacB and TthIII/npr-sacB PCR primers (SEQ ID NOs:9 and 10, respectively) to identify vectors containing the npr-sacB insert. The PCR products were analyzed on a 0.8% agarose gel. Plasmid DNA was isolated from cells containing the pGP704::sacB vector.

### Example 3

### Construction of pGP704::sacB::ΔCarotenoid White Mutant Strain

The present Example describes the creation of crt integration vectors that enable production of deletions within the native C₃₀ biosynthetic pathway of the Methylomonas genome (U.S. Ser. No. 10/997,844; hereby incorporated by reference). Specifically, a construct was made based on the positive selection vector pGP704::sacB that enable a chromosomal deletion within the crtN3 gene. Additionally, since the crtN1, ald, crtN2 genes (crtN1aldcrtN2 cluster) exist in an operon and these genes are co-transcribed from the same promoter, an additional construct was created that would permit deletion of the promoter for the crtN1aldcrtN2 cluster. These constructs (i.epGP704::sacB::ΔcrtN3, and pGP704::sacB::Δ promoter crtN1aldcrtN2 cluster) were generated using standard PCR and cloning methods, as described below.

PCR Amplification and Cloning of the Carotenoid Deletion DNA Fragments into pGP704::sacB.

For amplification of the subsequent PCR fragments [crtN3 deletion fragment #1 (˜1.2 kB), crtN3 deletion fragment #2 (˜1.1 kB), crtN1aldcrtN2 cluster promoter deletion fragment #1 (˜2.6 kB) and crtN1aldcrtN2 cluster promoter deletion fragment #2 (1.1 kB)], the following DNA primers (Table 3) were used, along with Methylomonas sp. 16a chromosomal DNA as template. The methodology used for PCR reactions and cloning into E. coli TOP10 One Shot™ cells were the same as previously described in Example 2. Several colonies from each transformation were screened for the proper insert DNA fragments using the QIAprep® Spin Mini-prep Kit for plasmid isolation.

TABLE 3
Primers Utilized for Cloning of the Carotenoid Deletion DNA Fraaments

| | | | |
|---|---|---|---|
| | | | Size of |
| Deletion | | | PCR |
| Fragment | Forward Primer | Reverse Primer | Fragment |

| | | | | | |
|---|---|---|---|---|---|
| aldehyde | Bg/II/aldehyde | (SEQ ID NO:31) | SphI-XhoI/ | (SEQ ID NO:32) | ˜1.1 kB |
| deletion | (deletion) #1: | | aldehyde (deletion) #1 | | |
| fragment #1 | 5′AGATCTTTGCA | | 5′GCATGCCTCGAGTG | | |
| | ACGGGTATTCG | | CTATCGTCGTCATACT | | |
| | ACGAAGG3′ | | CAGGCTTTG3′ | | |
| crtN3 deletion | Bg/II/crtN3 | (SEQ ID NO:23) | Bg/II-NotI/crtN3 | (SEQ ID NO:24) | ˜1.2 kB |
| fragment #1 | (deletion) #1 | | (deletion) #1 | | |
| | 5′AGATCTCCGT | | 5′AGATCTGCGGCGCC | | |
| | TCTGTACACTGA | | CATTTGTTGCTGATAG | | |
| | TCC | | AATCCGGC3′ | | |
| | G3′ | | | | |
| crtN3 deletion | 5′NotI/crtN3 | (SEQ ID NO:25) | 3′NotI/crtN3 (deletion) #2 | (SEQ ID NO:26) | ˜1.1 kB |
| fragment #2 | (deletion) #2 | | 5′GCGGCCGCCGAATA | | |
| | 5′GCGGCCGCG | | CCTCGACATTCAAGC | | |
| | CAAGCCGGCCA | | 3′ | | |
| | ACAGGGATTCC | | | | |
| | 3′ | | | | |
| crtN1aldcrtN2 | Bg/II(truncated | (SEQ ID NO: 27) | SphI(promoter deletion): | (SEQ ID NO:28) | ˜2.6 |
| cluster | crtN1): | | 5′GCATGCCGACATCTA | | |
| promoter | 5′AGATCTAACT | | GTTGTCCAGC3′ | | |
| deletion | GTGCGAGCGCC | | | | |
| fragment #1 | GTAGC3′ | | | | |
| crtN1aldcrtN2 | Bg/II(promoter | (SEQ ID NO:29) | NotI(promoter deletion): | (SEQ ID NO:30) | ˜1.1 kB |
| cluster | deletion): | | 5′GCGGCCGCTGTCGT | | |
| promoter | 5′AGATCTTGGC | | GCGAATGCATCAGC3′ | | |
| deletion | GCTTGATCGAA | | | | |
| fragment #2 | ATCGTCG3′ | | | | |

**Underlined sequences represent restriction endonuclease recognition sites.

Construction of Integration Vector pGP704::sacB::ΔcrtN3

The re-NSI (replacement-Nucleotide Sequence of Interest) used to delete the crtN3 gene from the Methylomonas genome was generated by ligating two PCR fragments (i.e., crtN3 deletion fragment #1 and crtN3 deletion fragment #2) into pGP704::sacB. Through ligating these two fragments, a deletion is produced in the crtN3 gene.

The crtN3 deletion fragment #1 (˜1.1 kB) was excised from the pCR2.1 (TOPO TA vector) by restriction digestion with BamHI and XhoI. The restriction digestion mixture was separated on a 0.8% agarose gel and the crtN3 deletion fragment #1 was extracted using the Qiaquick® Gel Extraction Kit (Qiagen). This BamHI and XhoI fragment was then digested with Bg/II and was ligated into the Bg/II site of dephosphorylated pGP704::sacB. After an overnight incubation at room temperature, the ligation mixture was used to transform calcium chloride competent E. coli SY327 cells (Miller, V. L. and Mekalanos, J. J., supra). The transformation mixture was plated onto LB+Amp²⁵ agar plates. Individual colonies were screened for the appropriate insert DNA using PCR methodology and PCR primers Bg/II/crtN3 (deletion) #1 (SEQ ID NO:23) and Bg/II-NotI/crtN3 (deletion) #1 (SEQ ID NO:24) with plasmid DNA as template. Plasmid DNA was isolated from the positive clones, pGP704::sacB::crtN3 deletion fragment #1.

The crtN3 deletion fragment #2 was isolated from the TOPO TA vector by digestion with EcoRI and was separated on a 0.8% agarose gel. The ˜1.1 kB DNA fragment was extracted from the gel using the Qiaquick® Gel Extraction Kit. The crtN3 deletion fragment #2 was digested with NotI and ligated into the dephosphorylated NotI site of pGP704::sacB::crtN3 deletion fragment #1. The ligation mixture was used to transform E. coli SY327 cells. Several colonies were screened using PCR methodology (Perkin Elmer AmpliTaq® and Epicentre Fail-Safe™ enzymes) using the Bg/II/crtN3 (deletion) #1 (SEQ ID NO:23) and the 3′NotI/crtN3 (deletion) #2 (SEQ ID NO:26) primers and plasmid template DNA. By using the forward primer for fragment #1 and the reverse primer for fragment #2, the desired plasmid with the two fragments in the same orientation was identified. Plasmid DNA was isolated from the positive clone and digested with M/uI and NdeI to confirm the presence of the correct insert DNA fragment. E. coli cells containing pGP704::sacB::ΔcrtN3 were streaked onto fresh medium to obtain isolated colonies.

Construction of Integration Vector pGP704::sacB::Δ promoter crtN1aldcrtN2

To prepare for the construction of the crtN1aldcrtN2 cluster promoter deletion vector (pGP704::sacB::Δ promoter crtN1aldcrtN2 cluster), an intermediary vector was generated, pGP704::sacB::hybrid. The components of pGP704::sacB::hybrid were pGP704::sacB, aldehyde deletion fragment #1 and crtN3 deletion fragment #2. The purpose of this vector was to make it easier to distinguish between fragments that had been cut with two restriction endonucleases as opposed to only one. This can be visualized on an agarose gel with the presence of an ˜1.1 kB fragment when digested with Bg/II and SphI.

The Bg/II and SphI digested pGP704::sacB::hybrid was ligated with the crtN1aldcrtN2 cluster promoter deletion fragment #1 (˜2.6 kB) which had been prepared using methods similar to those described above. The ligation mixture was used to transform E. coli SY327 cells and the transformation mixture was plated onto LB+Amp²⁵ agar plates. Colonies containing the correct insert DNA fragment were identified though screening using plasmid isolation, restriction digestion and agarose gel electrophoresis.

The pGP704::sacB:: ΔcrtN1aldcrtN2 cluster promoter deletion fragment #1 was digested with Bg/II and NotI, separated on a 0.8% agarose gel and extracted from the agarose gel using the Qiaquick® Gel Extraction Kit. The Bg/II and NotI digested pGP704::sacB::ΔcrtN1aldcrtN2 cluster promoter deletion fragment #1 was ligated with the crtN1aldcrtN2 cluster promoter deletion fragment #2. The ligation mixture was used to transform E. coli SY327 cells and was plated onto LB+Amp²⁵ agar plates as described above. Colonies containing the correct insert DNA fragment were identified by plasmid isolation and restriction digestions using methods similar to those described above. Cells containing positive vectors (pGP704::sacB::Δ crtN1aldcrtN2 cluster promoter) were streaked for isolated colonies. Through ligating the crtN1aldcrtN2 cluster promoter deletion fragment #1 and #2, a deletion of is produced in the crtN1aldcrtN2 gene cluster promoter.

### Example 4

### Tri-Parental Conjugation of crt Integration Vector Into Methylomonas sp. 16a

The crt integration vector pGP704::sacB::Δ crtN1aldcrtN2 cluster promoter from Example 3 was transferred into Methylomonas sp. 16a via triparental conjugation. Specifically, the following were used as recipient, donor, and helper, respectively: Methylomonas sp. 16a, E. coli SY327 containing the crt integration vector, and E. coli containing pRK2013 (ATCC No. 37159).

Theory of the Conjugation

The mobilization of vector DNA into Methylomonas occurs through conjugation (tri-parental mating). The pGP704::sacB vector used to make chromosomal mutations in Methylomonas has a R6K origin of replication, which requires the π protein. This vector can replicate in E. coli strain SY327, which expresses the π protein. However, this protein is not present in the Methylomonas genome. Therefore, once the vector DNA has entered into Methylomonas, it is unable to duplicate itself. If the vector also contains a DNA segment that shares homology to a region of the Methylomonas genome, the vector can be integrated into the host's genome through homologous recombination. The homologous recombination system of Methylomonas appears to be similar to that of other Gram-negative organisms.

In the case of Methylomonas, the mobilizable plasmid (pGP704::sacB) was used to transfer re-NSI into this bacterium. The conjugative plasmid (pRK2013; ATCC No. 37159), which resided in a strain of E. coli, facilitated the DNA transfer.

Growth of Methylomonas sp. 16a

The growth of Methylomonas sp. 16a for tri-parental mating initiated with the inoculation of an −80° C. frozen stock culture into 20 mL of BTZ medium containing 25% methane, as described in Example 1. The culture was grown at 30° C. with aeration until the density of the culture was saturated. This saturated culture was in turn used to inoculate 100 mL of fresh BTZ medium containing 25% methane. The 100 mL culture was grown at 30° C. with aeration until the culture reached an OD₆₀₀ between 0.7 and 0.8. To prepare the cells for the tri-parental mating, the Methylomonas sp. 16a cells were washed twice in an equal volume of BTZ medium. The Methylomonas cell pellets were re-suspended in the minimal volume needed (approximately 200 to 250 μL). Approximately 40 μL of the re-suspended Methylomonas cells were used in each tri-parental mating experiment.

Growth of the Escherichia coli Donor and Helper Cells

Isolated colonies of the E. coli donor (pGP704::sacB::Δ crtN1aldcrtN2 cluster promoter) and helper (containing conjugative plasmid pRK2013) cells were used to separately inoculate flasks with 5 mL of LB broth containing 25 μg/μL Kan. These cultures were grown overnight at 30° C. with aeration. The following day, the E. coli donor and helper cells were mixed together and incubated at 30° C. for ˜2 hr. Subsequently, the cells were washed twice in equal volumes of fresh LB broth to remove the antibiotics.

Tri-parental Mating: Mobilization of the Donor Plasmid into Methylomonas Strain 16a

Approximately 40 μL of the re-suspended Methylomonas cells were used to re-suspend the combined E. coli donor and helper cell pellets. After thoroughly mixing the cells, the cell suspension was spotted onto BTZ agar plates containing 0.05% yeast extract. The plates were incubated at 30° C. for 3 days in a jar containing 25% methane.

Following the third day of incubation, the cells were scraped from the plate and re-suspended in BTZ broth. The entire cell suspension was plated onto several BTZ agar plates containing Amp³⁵. The plates were incubated at 30° C. in a jar containing 25% methane until colonies were visible (˜4-7 days).

Individual colonies were streaked onto fresh BTZ+Amp³⁵ agar plates and incubated 1-2 days at 30° C. in the presence of 25% methane. These cells were used to inoculate bottles containing 20 mL of BTZ and 25% methane. After overnight growth, 5 mL of the culture was concentrated by centrifugation using a tabletop centrifuge. Then, to rid the cultures of E. coli cells that were introduced during the tri-parental mating, the cells were inoculated into 20 mL of BTZ liquid medium containing nitrate (10 mM) as the nitrogen source, methanol (200 mM), and 25% methane and grown overnight at 30° C. with aeration. Cells from the BTZ (nitrate) cultures were again inoculated into BTZ and 25% methane and grown overnight at 30° C. with aeration. The cultures were monitored for E. coli growth by plating onto LB agar plates to verify the success of the E. coli elimination.

### Example 5

### Evaluation of Methylomonas Transconjugants Containing the crtN1aldcrtN2 Cluster Promoter Deletion Integration Vector

Following the mobilization of the crtN1aldcrtN2 cluster promoter deletion integration construct into Methylomonas sp. 16a, as described in Example 4, a two-step selection strategy was applied as described below to identify the Δ crtN1aldcrtN2 cluster promoter allelic exchange mutants. A “white” or “pigmentless” mutant was produced comprising the Δ crtN1aldcrtN2 cluster promoter.

Preliminary Screening for Allelic Exchange Mutants

Cultures free of E. coli cells were passaged several times in fresh medium (1 mL of culture into 20 mL of fresh BTZ medium), to increase the probability of occurrence of a second crossover event. Subsequently, cells were plated onto BTZ and sucrose (5%) agar plates. Those cells grown on plates containing sucrose had lost the integration vector, which contained the sacB gene. However, the loss of the vector sequences could be due to the second crossover event occurring either on the same or opposite side of the re-NSI that was present on the insert DNA. If the second crossover event had occurred on the same side of the re-NSI as the first crossover event, the wildtype gene of interest would be regenerated. In contrast, if the second crossover event occurred on the opposite side of the re-NSI as the first crossover event, the deletion of the gene of interest would be established in the Methylomonas genome.

Verification of the Chromosomal Deletion of the Methylomonas sp. 16a ΔcrtN1aldcrtN2 Cluster Promoter

Chromosomal DNA was purified from several cultures that had grown on the sucrose plates using the MasterPure™ DNA Purification Kit (EPICENTRE®; Madison, Wis.). Then, PCR amplification methods were applied to confirm each suspected deletion, using the primers described below in Table 4.

TABLE 4

Primers Used to Verify the Deletion of the Methylomonas sp.
16a crtN1aldcrtN2 gene cluster promoter

| | | | | |
|---|---|---|---|---|
| Carotenoid | | | Intact | Deletion |
| Gene | Forward Primer | Reverse Primer | Fragment | Fragment |

| | | | | | | |
|---|---|---|---|---|---|---|
| crtN1ald | Bg/II | (SEQ ID NO: 27) | NotI | (SEQ ID NO:30) | ˜4.3 kB | ˜2.1 kB |
| crtN2 | (truncated crtN1): | | (promoter deletion) | | | |
| cluster | 5′-AGATCTAACT | | 5′-GCGGCCGCTG | | | |
| promoter | GTGCGAGCGCC | | TCGTGCGAATGC | | | |
| | GTAGC-3′ | | ATCAGC-3′ | | | |

**Underlined sequences represent restriction endonuclease recognition sites.

Δ crtN1aldcrtN2 Cluster Promoter Mutant Phenotype

The Methylomonas strain with the Δ crtN1aldcrtN2 cluster promoter had a “white” phenotype, was designated herein as MWM100, and was easily distinguished from the wild-type cells. However, the construction of this strain was still verified via PCR amplification using PCR primers Bg/II (truncated crtN1) (SEQ ID NO:27) and NotI (promoter deletion) (SEQ ID NO:30). Cells that contained an intact promoter region for the crtN1aldcrtN2 cluster had the expected PCR product size of ˜4.3 kB. In contrast, cells in which the promoter region of the crtN1aldcrtN2 cluster had been deleted, gave rise to PCR products that were −2.1 kB (Table 4).

### Example 6

### Combination of crtN3 Deletion with crtN1aldcrtN2 Cluster Promoter Deletion in Methylomonas

Addition of crtN3 Deletion Mutation to Δ crtN1aldcrtN2 Cluster Promoter Strain

The pGP704::sacB::ΔcrtN3 integration plasmid described above was transferred into MWM1100 via conjugation using the same procedures described above in Example 4. Once inside the Methylomonas, the crtN3 gene was deleted via homologous recombination using the same two step strategy described in Example 5. The deletion of the crtN3 gene was confirmed using PCR methodology and PCR primers Bg/II/crtN3 (deletion) #1(SEQ ID NO:23) and 3′ NotI/crtN3 (deletion) #2 (SEQ ID NO:26) (Table 3). If the “white” mutants still contained the intact crtN3 gene, a PCR fragment that was −3.5 kB was produced. In contrast, cells in which the crtN3 gene was deleted produced an ˜2.3 kB PCR fragment (Table 4). The new Methylomonas strain that was produced is referred to herein as MWM1200 (ΔcrtN1aldcrtN2 cluster promoter+ΔcrtN3). This is the parent strain for integration of crt genes cluster via homologous recombination.

### Example 7

### Construction of Vectors for Integrating Genes in Methylomonas

Integration vectors for Methylomonas are those that can be transferred into, but cannot replicate in this organism. Common vectors used in E. coli can be modified for this purpose by the insertion of a transfer region. Vector pGP704 has been used for deletions as described before. However, cloning of gene clusters involved in canthaxanthin production into this vector can sometimes be problematic. In this experiment, a medium copy number plasmid, pTrcHis2 (Invitrogen) and a low copy number vector, pACYC, were chosen as the backbones for the construction of new integration vectors. The following elements were added to these vectors: a multiple cloning site, mob region for gene transfer from pGP704, Kan resistance marker for antibiotic selection and sacB gene for sucrose selection.

Modification of pTrcHis2 Vector

The first step of the modification was the introduction of a polylinker containing several unique restriction sites. This linker (SEQ ID NOs:44 and 45) contained an MfeI site on each end and internal NotI, SpeI, XbaI, EcoRI Bg/II, BamHI, KpnI and PacI sites. The linker oligonucleotides wwere annealed and cut with MfeI, then cloned into the EcoRI site of pTrcHis2.

SEQ ID NO:44

5′-ATCCAATTGGCGGCCGCGACTAGTTCTAGACGAATTCAGATCTTTAA
TTAAGGATCCGGTACCGCGGCCGCCAATTGATC-3′:

SEQ ID NO:45

5′-GATCAATTGGCGGCCGCGGTACCGGATCCTTAATTAAAGATCTGAAT
TCGTCTAGAACTAGTCGCGGCCGCCAATTGGAT-3′:

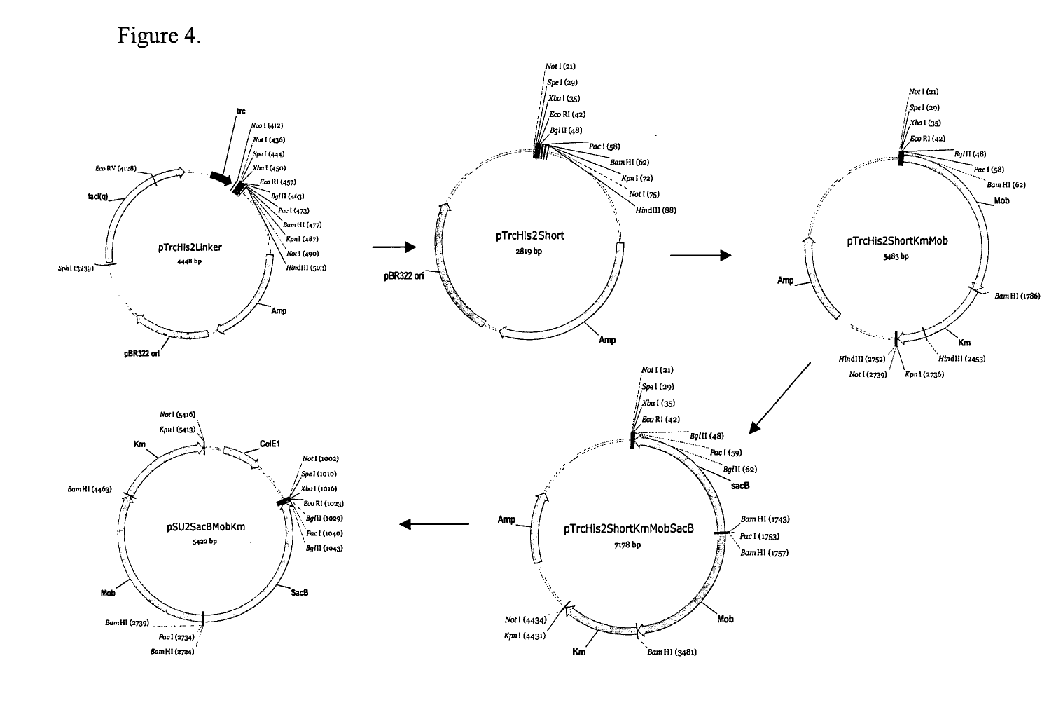

Plasmids containing the linker were identified by restriction enzyme digestion and gel electrophoresis. The orientation of the inserted polylinker was determined by the release of a 53 bp Hind/III and XbaI fragment and a plasmid with the linker inserted such that the XbaI-EcoRI-Bg/II sites are in the same direction as the amp coding region was called pTrcHis2Linker (FIG. 4). The Hind/III site was from the original vector, but not part of the linker.

This resulting vector was further modified by deleting the SphI to NcoI region. This was done by cutting the vector with these two restriction enzymes, followed by removal of the overhang regions with mungbean nuclease using the conditions recommended by the manufacture (New England Biolab) and subsequent ligation. The resulting vector was named pTrchis2Short (FIG. 4). The kanamycin resistance gene (including the promoter) was PCR amplified using standard conditions (Example 2) from vector pBHR1 (MoBiTec GmbH, Goettingen, Germany) using primers Kam F-SpeBgI and KamR-speBamHI (SEQ ID NOs:46 and 47).

SEQ ID NO:46

5′-GACTAGTAGATCTTCTGATTAGAAAAACTCATCGAGCA-3′:

SEQ ID NO:47

5′-GACTAGTGGATCCGGAAAGCCACGTTGTGTCTCAAAATC-3′:

The PCR product was cut with Bg/II and BamHI and cloned into the BamHI site of pTrcHis2Short. A resulting plasmid with the Kan coding region in the same orientation as the Amp coding region was identified by restriction enzyme digestion followed by gel electrophoresis and chosen for further use. The 1.7 kB mob transfer region was isolated from pGP704 (V. L. Miller, V. L., and Mekalanos, J. J., J. Bacteriol., 170:2575-2583 (1988)) as a BamHI fragment and cloned into the BamHI site next to the Kan resistance marker. A plasmid with the mob fragment in the same orientation as the kan coding region was identified by restriction enzyme digestion followed by gel electrophoresis and this resulting construct was named pTrchis ShortKmMob (FIG. 4).

Cloning of the npr-sacB Gene

The npr-sacB gene was amplified from pGP704::sacB, constructed in EXAMPLE 2, with primers SacB F-PacBamHIand SacBR-PacIBgI (SEQ ID NOs:48 and 49) using the PCR conditions described in Example 2.

SEQ ID NO:48

5′-CCTTAATTAAGGATCCGATCTTAACATTTTTCCCCTATCATT-3′:

SEQ ID NO:49

5′-CCTTAATTAAGATCTGTTATTAGTTGACTGTCAGCTGTC-3′:

The PCR product was cut with the restriction enzyme PacI and cloned in the corresponding site in the polylinker of pTrcHisKmMob. A plasmid with the npr-sacB gene in the opposite orientation to the kan coding region was identified by restriction enzyme digestion and gel electrophoresis. The resulting construct was named pTrchis ShortKmMobSacB (FIG. 4).

Modification of Low Copy Number Plasmid pACYC

In additional to pTrcHis2, the low copy number plasmid pACYC was also modified to create an integration vector. The origin of replication for this plasmid was amplified using standard conditions (Example 2) with primers that each incorporated a NotI site: pSU NotI Rev and pSU NotI For 2 (SEQ ID NOs:50 and 51).

SEQ ID NO:50

5′-ATTTGCGGCCGCCATACGAGCCGGAAGCATAAAGTG-3′:

SEQ ID NO:51

5′-ATTTGCGGCCGCCTGATTAATAAGATGATCTTCTTG-3′:

The PCR product was ligated with the NotI fragment from pTrchis ShortKmMobSacB containing the Kan resistance marker, mob region and npr-sacB gene. The resulting integration vector was named pSUSacBMobKm.

### Example 8

### Construction of Vector Containing crtEWYIB Gene Cluster

Our objective was to identify chromosomal regions in Methylomonas that could support a level of gene expression that would result in a high level of canthaxanthin production. The strategy was to randomly integrate the promoterless crtEWYIB gene cluster into the chromosome and screen for high canthaxanthin production.

Construction of crtEWYIB Cluster

Pantoea stewartii ATCC #8199 (WO 03/016503 corresponding to U.S. Ser. No. 10/218,118; hereby incorporated by reference) contains the natural gene cluster crtEXYIBZ. The genes required for β-carotene synthesis (i.e., crtE and crtYIB) were joined together by PCR. Specifically, the crtE gene (SEQ ID NO: 35) and crtYIB genes (SEQ ID NO: 36) were each amplified using chromosomal DNA as template and the primers given in Table 5.

TABLE 5
Primers Used for Creation of the crtEYIB Reporter Construct

| | | |
|---|---|---|
| Gene(s) | Forward Primer | Reverse Primer |

| | | | | |
|---|---|---|---|---|
| crtE | pBHRcrt_1F: | (SEQ ID NO: 37) | pBHRcrt_1R: | (SEQ ID NO: 38) |
| | 5′-GAATTCGCCCTTGACG | | 5′-CGGTTGCATAATCCTGCC | |
| | GTCT-3′ | | CACTCAATTGTTAACTGACGGCA | |
| | | | GCGAGTTTT-3′ | |
| crtYIB | pBHRcrt_2F: | (SEQ ID NO: 39) | pBHRcrt_2R: | (SEQ ID NO: 40) |
| | 5′-AAAACTCGCTGCCGTC | | 5′-GGTACCTAGATCGGGC | |
| | AGTTAACAATTGAGTGGGC | | GCTGCCAGA-3′ | |
| | AGGATTATGCAACCG-3′ | | | |

Note:
Underlined portions within each primer correspond to restriction sites for EcoRI, MfeI.

The PCR reactions were performed with Pfu DNA polymerase in buffer supplied by the manufacturer containing dNTPs (200 μM of each). Parameters for the thermocycling reactions were: 92° C. (5 min), followed by 30 cycles of: 95° C. (30 sec), 55° C. (30 sec), and 72° C. (5 min). The reaction concluded with 1 cycle at 72° C. for 10 min. The two PCR products were gel purified and joined together by a subsequent PCR reaction using the primers pBHRcrt_{—}1F (SEQ ID NO:37) and pBHRcrt_{—}2R (SEQ ID NO:40). Parameters for the thermocycling reaction were: 95° C. (5 min), followed by 20 cycles of: 95° C. (30 sec), 55° C. (1 min) and 72° C. (8 min). A final elongation step at 72° C. for 10 min completed the reaction. The final 4511 bp PCR product was cloned into the pTrcHis2-Topo vector (Invitrogen, Carlsbad, Calif.) in the forward orientation, resulting in plasmid pDCQ300. The ˜4.5 kB EcoRI fragment of pDCQ300 containing the crtEYIB gene cluster was ligated into the unique EcoRI site of vector pBHR1 (MoBiTec GmbH, Goettingen, Germany), to create construct pDCQ301. In pDCQ301, a unique MfeI site was engineered in the intergenic region of crtE and crtY through the primers in the procedure described above.

A codon optimized crtW gene was added to the crtEYIB gene cluster. The sequence of the crtW gene from Agrobacterium aurantiacum was optimized for expression in Methylomonas sp. 16a by altering codons to those most commonly found in highly expressed Methylomonas sp. 16a genes (U.S. Ser. No. 10/997,844 hereby incorporated by reference). Additionally, most strong hairpin structures were disrupted by replacement with alternative sub-optimal codons. The AT-rich mRNA instability region (Guhaniyogi, G. and J. Brewer, Gene 265(1-2):11-23 (2001)) and the long runs of the same nucleotide were also eliminated. In the case of a string of more than 3 or 4 of the same amino acids, a sub-optimal codon was also introduced to prevent shortage of the most preferred codon pool for this amino acid. The ribosomal binding site (RBS) was engineered upstream of the start codon as the RBS sequence from pTrcHis2-TOPO vector (Invitrogen). Several restriction sites were also engineered at the 5′ and 3′ ends of the gene to facilitate cloning. The resulting designed crtW gene sequence (SEQ ID NO: 7) was synthesized by Aptagen Inc. (Herndon, Va.) and cloned onto the pCRScript vector to form pCRScript-Dup1. There is 84% nucleotide identity between the native gene (SEQ ID NO: 8) and the synthetic gene, with no changes in the encoded amino acid sequence (SEQ ID NO:33).

The ˜0.8 kB EcoRI fragment of pCRScript-Dup1 containing the synthetic codon-optimized crtW gene was ligated to the unique MfeI site in pDCQ301. In the resulting construct pDCQ307, the crtEWYIB genes were under the control of the chloramphenicol resistance gene promoter of the vector.

The 5.3 kB EcoRI fragment containing the crtEWYIB region was isolated from pDCQ307 and cloned into the EcoRI site in the integration vector pTrcH is ShortKmMob. A plasmid with the EcoRI fragment inserted such that the coding regions were in opposite orientation to the kan coding region was identified by restriction digestion and gel electrophoresis. Genomic DNA fragments of Methylomonas sp. 16a ranging from about 1 to 2 kB were obtained by Sau3A partial digestion and gel purification. These fragments were then cloned into the Bg/II site immediately upstream from the crtEWYIB cluster in pTrcH is ShortKmMob creating a library of random genomic fragments, using E. coli as the host.

### Example 9

### Integration of the crtEWYIB Gene Cluster Through Single-Crossover Using the Genomic Fragment Library

The library of random genomic fragments inserted in the pTrchis ShortKmMob vector also containing crtEWYIB was transferred from E. coli into Methylomonas by triparental conjugation as described in Example 4. The helper strain was E. coli containing pRK2013. The Methylomonas sp. 16a recipient strain was the white mutant strain MWM1200 described in Examples 2-6.

The presence of the Methylomonas sp. 16a genomic DNA fragments allowed the integration of the entire vector into the recipient genome through a single-crossover event. The integration was directed by the homology between the genomic DNA fragment within the vector and the same sequence in the genome. Thus the integration was expected to occur at a location in the genome adjacent to the genomic DNA fragment insert sequence. After conjugation, colonies were grown on BTZ medium containing 50 μg/mL kanamycin, and visually screened for the presence of canthaxanthin, which is seen as an orange color. Approximately 400 (only about 400 due to its low frequency) colonies were screened. Three colonies had a strong orange color indicating production of high levels of canthaxanthin. These three colonies, L1, L2, and L6, were selected for further investigation.

### Example 10

### Identification of Chromosmal Integration Sites

To locate the insertion sites of the crtEWYIB gene cluster for the three clones selected in Example 9, the genomic DNA from each of these strains was isolated. Genomic DNA was prepared using the Fast DNA Kit (Bio 101; Carlsbad, Calif.). A single primer amplification procedure (Karlyshev et al., BioTechniques, 28:1078-1081 (2000)) was used to amplify the chromosomal DNA region upstream from the first gene (crtE) of the integrated crtEWYIB gene cluster for each of L1, L2, and L6 samples. The amplification primer was CrtE-Chrom (SEQ ID NO:34) which is located at the 5′ end of crtE.

SEQ ID NO:34 5′-TGCCCGGTGCCAGCGTGCCTTC-3′:

The single primer amplification procedure consisted of three rounds of amplification. The first round was for linear amplification of single-stranded DNA with the CrtE-Chrom primer. This step consists of 30 cycles and was carried out at a standard annealing temperature (94° C., 30 sec; 50° C., 30 sec; and 72° C. for 3 min). The second round of amplification involved a low annealing temperature (30° C.). Other amplification conditions were the same as the first round. The purpose of this round was to obtain a mixture of specific and non-specific double stranded DNA. The third round of PCR amplification was carried out to further enrich the specific PCR products from round two. The amplification conditions were the same as the first round with a final extension period added (72° C. for 7 min).

The PCR products were then sequenced with two other primers: CrtE1 and CrtE2 (SEQ ID NOs:52 and 53). These sequencing primers were designed based on the 5′-end DNA sequence of the crtE coding region upstream from the CrtE-Chrom primer.

| | |
|---|---|
| SEQ ID NO:52 | 5′- AGTAACTGATCAAGGCGGCTATCG-3′: |
| SEQ ID NO:53 | 5′- TATCGATATCAGCCAGCAACTGC-3′: |

The integration sites for L1, L2, and L6 in the Methylomonas sp. 16a chromosome were identified by comparing the sequence of each amplified adjacent fragment to the total genomic sequence In both L1 and L2 strains, the crtEWYIB gene cluster along with the vector was inserted in the putative Ion gene (FIG. 2); an ORF (SEQ ID NO:17) encoding a protein (SEQ ID NO:18) with amino acid sequence similarity to the Lon protease. The L6 insertion was at a different location, and is not included in this invention. Further sequence analysis of the L1 and L2 insertion region showed that this putative Ion gene is one ORF in a gene cluster that includes six ORFs that all appear to be involved in protein metabolism. The first ORF (SEQ ID NO:11) of this cluster encodes a protein (SEQ ID NOL 12) with sequence similarity to trigger factor, tig. The second ORF (SEQ ID NO:13) in the cluster encodes a protein (SEQ ID NO:14) with similarity to clpP, the third ORF (SEQ ID NO:15) in the cluster encodes a protein (SEQ ID NO:16) with similarity to clpX, the fourth ORF (SEQ ID NO:17) in the cluster encodes a protein (SEQ ID NO:18) with similarity to Ion, the fifth ORF (SEQ ID NO:19) in the cluster encodes a protein (SEQ ID NO:20) with similarity to himA, and the sixth ORF (SEQ ID NO:21) in the cluster encodes a protein (SEQ ID NO:22) with similarity to ppiC. This tig-clpP-clpX-lon-himA-ppiC gene cluster including non-coding sequences between the ORFs, herein called the Tig region (SEQ ID NO:1), was chosen as a target region of the Methylomonas sp. 16a genome for integration of genes to obtain high levels of expression.

### Example 11

### Constructions for Integration of the crtEWYIB and crtWIdiEYIB Gene Clusters Through Double-Crossover

Random integration via single-crossover as decribed in examples 9 and 10 serves the purpose of identifying chromosomal regions that can support expression of the gene of interests. However, these single crossover strains requires selection with antibiotics. To obtain a stable strain that do not contain the antibiotic marker, double-crossover recombination was used to integrate the crt EWYIB and crtWIdiEYIB gene clusters in the tig region.

Construction of Integration Vectors

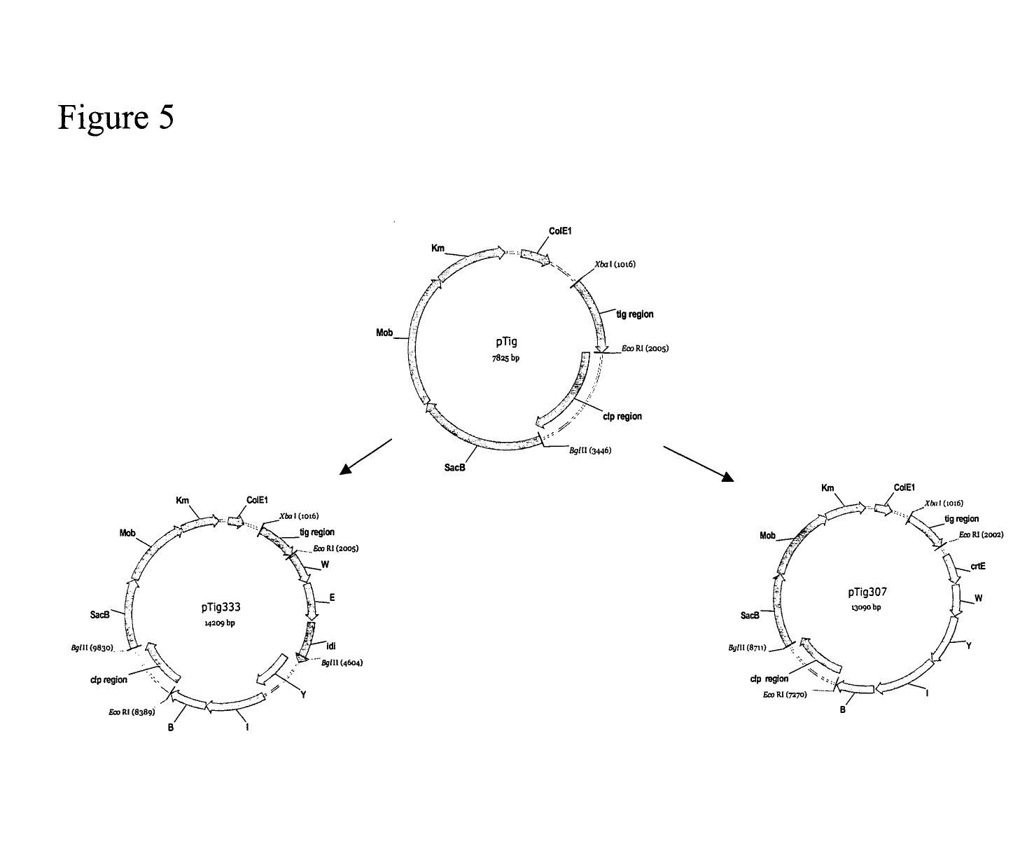

Due to its proximity to the promoter, the DNA region between the putative tig and clpP genes in the Methylomonas chromosome was used as a target for integration of two different crt gene clusters through double-crossover recombination. DNA regions from tig and clpP genes were used as the homology regions (h-NSI) in preparing the integration vectors pTig307 and pTig333 (FIG. 5). Both vectors were constructed based on the vector pSUSacBMobKm that was described in Example 7. The two homology regions were amplified by a sewing PCR method. In the first step, an approximately 1.0 kb DNA region from the tig gene was amplified with primers Trigger For XbaI and Trigger RevEcorl (SEQ ID NOs:54 and 55), Primer Trigger For XbaI was designed based on DNA sequences about 1 kB upstream from the end of the tig coding region and an XbaI site was included in the primer. Primer Trigger RevEcorl was designed based on DNA sequence at the end of the ORF of the tig gene and an EcoRI restriction site was included. The second homology region was about 1.4 kB and contained the clpP gene and a portion of clpX. This region was amplified with the primers Trigger ForEcorl and Trigger Rev BgII (SEQ ID NOs:56 and 57). The 5′ end of primer Trigger ForEcorl was complimentary to the entire sequence of primer Trigger RevEcorl. The primer Trigger Rev BgIII was designed based on a DNA sequence in the clpX gene and the restriction enzyme site Bg/II was added for cloning purposes.

SEQ ID NO:54

5′-GCTCTAGAGAAGTTTACCCTGAAATCGGTCTG-3′:

SEQ ID NO:55

5′-GAATTCTTCCTATGCTTGCTGCCGTTCCATG-3′:

SEQ ID NO:56

5′-CATGGAACGGCAGCAAGCATAGGAAGAATTCACTGAATGATTGATCT
AACTGGCATG-3′:

SEQ ID NO:57

5′-GAAGATCTGCTGCGGATGCTTGCGTCCACCTTG-3′:

After gel purification, one-fourth of the PCR products from the above first two PCR reactions were combined for the second step of PCR (no primers added). PCR amplification conditions for second step were: 94° C., 2 min to denature the DNA followed by 10 cycles of amplification under the following conditions: 94° C., 30 sec; 50° C., 30 sec; 72° C. for 4 min. Then primer set Trigger For XbaI and Trigger Rev BgII (SEQ ID NOs:54 and 57) was added. The PCR reaction was allowed to proceed for another 25 rounds under the same conditions. The resulting PCR product contained DNA regions from both tig and clpP genes and was cloned into the XbaI and Bg/II sites in the pSUSacBMobKm vector creating pTig.

The crtEWYIB gene cluster described in Example 8 was isolated as an EcoRI fragment from pDCQ307, and cloned into the EcoRI site of pTig. This EcoRI site was created by the PCR described above, and lies between the tig and clpP genes. A plasmid with the crtEWYIB genes in the same orientation as the tig and clpP coding sequences was identified by restriction enzyme digestion and gel analysis. The resulting vector was called pTig307.

Construction of crtWEidiYIB Gene Cluster

The crtWEidiYIB gene cluster containing natural crtEidiYIB genes and the codon optimized crtW gene was prepared as follows. The carotenoid synthesis gene cluster crtEidiYIBZ (SEQ ID NO:41), as described by Cheng in copending U.S. patent application Ser. No. 10/808,807, was isolated from the environmental isolate P. agglomerans DC404. The soil from a residential vegetable garden in Wilmington, Del. was collected and resuspended in LB medium. A 10 μL loopful of resuspension was streaked onto LB plates and the plates were incubated at 30° C. Pigmented bacteria with diverse colony appearances were picked and streaked twice to homogeneity on LB plates and incubated at 30° C. From these colonies, one that formed pale yellow smooth translucent colonies was designated as “strain DC404”.

P. agglomerans strain DC404 was grown in 25 mL of LB medium at 30° C. overnight with aeration. Bacterial cells were centrifuged at 4,000×g for 10 min. The cell pellet was gently resuspended in 5 mL of 50 mM Tris-10 mM EDTA (pH 8.0) and lysozyme was added to a final concentration of 2 mg/mL. The suspension was incubated at 37° C. for 1 hr. Sodium dodecyl sulfate was then added to a final concentration of 1% and proteinase K was added at 100 μg/mL. The suspension was incubated at 55° C. for 2 hr. The suspension became clear and the clear lysate was extracted twice with an equal volume of phenol:chloroform:isoamyl alcohol (25:24:1) and once with chloroform:isoamyl alcohol (24:1). After centrifuging at 4,000 rpm for 20 min, the aqueous phase was carefully removed and transferred to a new tube. Two volumes of ethanol were added and the DNA was gently spooled with a sealed glass Pasteur pipet. The DNA was dipped into a tube containing 70% ethanol. After air drying, the DNA was resuspended in 400 μL of TE (10 mM Tris-1 mM EDTA, pH 8.0) with RNaseA (100 μg/mL) and stored at 4° C. The concentration and purity of DNA was determined spectrophotometrically by OD₂₆₀/OD₂₈₀.

A cosmid library of DC404 was constructed using the pWEB cosmid cloning kit from Epicentre (Madison, Wis.) following the manufacturer's instructions. Genomic DNA was sheared by passing it through a syringe needle. The sheared DNA was end-repaired and size-selected on low-melting-point agarose by comparison with a 40 kB standard. DNA fragments approximately 40 kB in size were purified and ligated into the blunt-ended cloning-ready pWEB cosmid vector. The library was packaged using ultra-high efficiency MaxPlax Lambda Packaging Extracts, and plated on EPI100 E. coli cells. Two yellow colonies were identified from the cosmid library clones. The cosmid DNA from the two clones had similar restriction digestion patterns. This cosmid DNA, referred to herein as pWEB-404, contained the crtWEidiYIBZ gene cluster, given as SEQ ID NO: 41.

Primers pWEB404F: 5′-GAATTCACTAGTCGAGACGCCGGGTACCAACCAT-3′ (SEQ ID NO:42) and pWEB404R: 5′-GAATTCTAGCGCGGGCGCTGCCAGA-3′ (SEQ ID NO:43) were used to amplify a fragment from DC404 containing the crtEidiYIB genes (SEQ ID NO:6) by PCR. Cosmid DNA pWEB-404 was used as the template with PfuTurbo™ polymerase (Stratagene, La Jolla, Calif.), and the following thermocycler conditions: 92° C. (5 min); 94° C. (1 min), 60° C. (1 min), 72° C. (9 min) for 25 cycles; and 72° C. (10 min). A single product of approximately 5.6 kB was observed following gel electrophoresis. Taq polymerase (Roche Appled Science, Indianapolis, Ind.) was used in a ten minute 72° C. reaction to add additional 3′ adenosine nucleotides to the fragment for TOPO® cloning into pTrcHis2-TOPO (Invitrogen, Carlsbad, Calif.). Following transformation to E. coli TOP10 cells, several colonies appeared bright yellow in color, indicating that they were producing a carotenoid compound. The gene cluster was then subcloned into the broad host range vector pBHR1 (MoBiTec, LLC, Marco Island, Fla.), and electroporated into E. coli 10G cells (Lucigen, Middletown, Wis.). The transformants containing the resulting plasmid pDCQ330 were selected on LB medium containing 50 μg/mL kanamycin. The PCR primers used generated a unique SpeI site upstream of crtE in pDCQ330.

The ˜0.8 kB EcoRI fragment of pCRScript-Dup1, prepared as described in Example 8, containing the synthetic, codon-optimized crtW gene was first blunt-ended and then ligated to pDCQ330, which was digested by SpeI and blunt-ended. In the resulting construct pDCQ333, the crtW gene (SEQ ID NO:7) was cloned upstream of and in the same orientation as the genes of the crtEidiYIB cluster, and the crtWEidiYIB genes were under the control of the chloramphenicol resistance gene promoter of the vector.

The crtWEidiYIB gene cluster was isolated as an EcoRI fragment from pDCQ333 and cloned into the EcoRI site of pTig. A plasmid with the crtWEidiYIB genes in the same orientation as the tig and clpP coding sequences was identified by restriction enzyme digestion and gel analysis. The resulting vector was called pTig333.

### Example 12

### Integration of the crtEWYIB and crtWIdiEYIB Gene Clusters Through Double-Crossover

The integration vectors pTig307 and pTig333 were each conjugated into Methylomonas sp. 16a strain MWM1200, described in Examples 2-6, through triparental matings as described in Example 4. After conjugation, colonies with a single-crossover were selected on BTZ medium plates containing 50 μg/mL kanamycin and confirmed by primer set: CrtE-Chrom (SEQ ID NO:34) and Trigger Chromosome Up (SEQ ID NO:58).

SEQ ID NO:58 5′- GCCCGCGGACAAAAGCGAAGG-3′:.

These single-crossover strains were then grown on BTZ medium without any antibiotic and subcultured several times before testing for kanamycin sensitivity by plating on sucrose plates (BTZ+0.5% sucrose, freshly prepared) without kanamycin. Strains that had lost the kanamycin resistance were expected to have undergone a second cross over recombination event, thereby eliminating the kan gene and all of the vector DNA other than the crt gene cluster that lies between the tig and clpP homology regions. Genomic DNA was isolated from those strains that did not grow on kanamycin and the double-crossover integration was confirmed by PCR using primers CrtB Chrom and Trigger Chromosome Down (SEQ ID NOs:59 and 60).

| | |
|---|---|
| SEQ ID NO:59 | 5′-AGTTACTTCCCGGATGAAGAC-3′: |
| SEQ ID NO:60 | 5′-AACAGAATATTGGCGGTATTC-3′:. |

After confirmation, the integration strains Tig333-16 and Tig307-164 were chosen for further characterization.

### Example 13

### Characterization of Carotenoid Production in the Integration Strains

The integration strains Tig333-16 and Tig307-164, obtained by double-cross over, were sensitive to kanamycin and the cells were orange in color. To further characterize the ability of these strains to produce canthaxathin, cells of each strain were grown in liquid BTZ medium and the canthaxanthin titer was measured.

Two 500 mL bottles containing ˜60 mL of BTZ and 25% methane were grown until saturation (˜24 hr) for each of the Methylomonas canthaxanthin-producing cultures to be analyzed. After growth, the cells were concentrated by centrifugation at 8000 rpm for 10 minutes. The pellet was then either frozen at −80° C. or processed directly following centrifugation. To the cell pellet was added 0.5 mL of 0.1 mm glass beads, 4 mL of ethanol and 6 mL of dicloromethane. This mixture was vortexed for ˜2 min, then centrifuged at 8000 rpm for 10 min. The supernatant was transferred to a new tube and dried under nitrogen. The residue was dissolved in 5 mL chloroform/hexane (4.5% chloroform). The sample was filtered with a 0.2 μm Gelman Teflon® syringe filter and analyzed using HPLC-photodiode array.

A Beckman System Gold® HPLC with Beckman Gold Nouveau Software (Columbia, Md.) was used for the study. The prepared extract (20 μL) was loaded onto a Brownlee, Sheri-silica (5 μm particles; internal diameter 4.6 mm; length 250 mm) column (Perkin Elmer). The flow rate was 1.5 mL/min. The mobile phase contained acetone, n-hexane and benzene at a ratio of 2:5:94. Each sample was run for 20 min. The spectral data was collected by a Beckman photodiode array detector (model 168) at 470 nm.

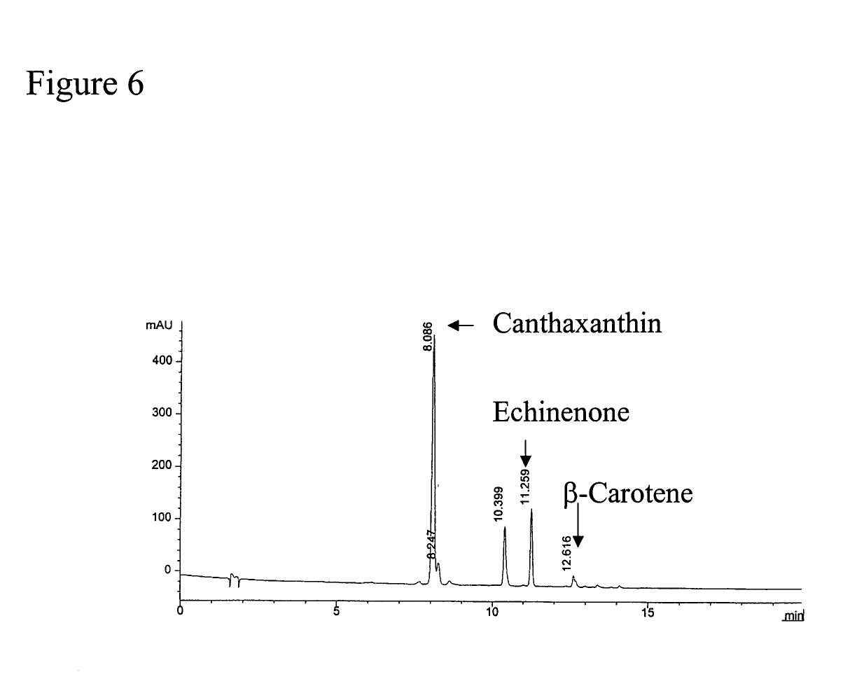

In the HPLC analysis of strain Tig333-16, shown in FIG. 6, a large peak representing cathaxanthin was present. The HPLC analysis of the Tig307-164 strain showed a similar profile. The titers for canthaxanthin produced by the Tig333-16 and Tig307-164 strains were about 629 and 700 ppm, respectively. Under the same growth conditions, approximately 900 ppm canthaxanthin was produced by multi-copy plasmid bearing strains. This result indicates that the single copy genes integrated in the tig region have very reasonable level of expression. More importantly, no antibiotic selection was necessary to maintain these double-crossover strains. Furthermore, based on continuous fermentation analysis (Example 15), the Tig333 strain is very stable.

### Example 14

### Continuous Fermentation of Methylomonas Strain Tig333-16

The ability of the Methylomonas sp. 16a integration strain Tig333-16 to produce canthaxnthin under fermentation conditions was tested as follows.

Cultures for inoculation of the fermenter were started from single colonies of Tig333-16 grown on initial fermentation media plates containing 17 g/L of agar.

TABLE 6
Initial Fermentation Media Composition

| | Amount | |
|---|---|---|
| Component | (g/L) | |
| NH₄Cl | 1.07 | |
| KH₂PO₄ | 1 | |
| MgCl₂*6H₂O | 0.4 | |
| CaCl₂*2H₂O | 0.2 | |
| 1M HEPES Solution (pH 7) | 50 | mL/L |
| Trace elements solution | 30 | mL/L |
| Na₂SO₄ | 1 | |

TABLE 7
Trace Elements Solution Composition for Initial Fermentation Media

| | Amount |
|---|---|
| Component | (g/L) |
| Nitrilotriacetic | 12.8 |
| acid | |
| FeCl₂*4H₂O | 0.3 |
| CuCl₂*2H₂O | 0.0254 |
| MnCl₂*4H₂O | 0.1 |
| CoCl₂*6H₂O | 0.312 |
| ZnCl₂ | 0.1 |
| H₃BO₃ | 0.01 |
| Na₂MoO₄*2H₂O | 0.01 |
| NiCl₂*6H₂O | 0.184 |

Colonies were inoculated into four 500 mL Wheaton bottles containing 65 mL of initial fermentation media and sealed with a butyl rubber stopper and aluminum crimp cap. Enough colonies were picked so as to give an initial optical density of 0.05 to 0.100. Methane was added to the culture by piercing the rubber stopper with a 60 mL syringe fitted with a 21 gauge needle to give a final methane concentration in the headspace of 25% (vol/vol). The inoculated medium was shaken for approximately 24 hr at 30° C. and 200 RPM in a controlled environmental rotary shaker. When cell growth reached saturation, 60 mL of each culture was used to inoculate the fermenter.

Continuous Fermentation

Continuous fermentations were performed under ammonia limitation using a 2 liter, vertical, stirred tank fermentor (B. Braun Biotech Inc., Allentown, Pa.) with a working volume of 1.6 liters. The fermentor was equipped with 3 six-bladed Rushton turbines and stainless steel headplate with fittings for pH, temperature, and dissolved oxygen probes, inlets for pH regulating agents, sampling tube for withdrawing liquid samples, and condenser. The fermentor was jacketed for temperature control with the temperature maintained constant at 30° C. through the use of an external heat exchanger. Dissolved oxygen was maintained constant at 10%+/−2% of air saturation at atmospheric pressure by feedback control using stirrer speed as the manipulated variable. The pH of the culture was maintained constant at 6.95 through the use of 5M NaOH as needed. Polypropylene glycol MW2000 was used at 0.405 mL/L to suppress foam formation. Slip streams were taken for the fermenter inlet and outlet gas lines for automated GC analysis of methane, O₂, and CO₂ concentrations using an Agilent Micro 3000 GC (Agilent Technologies Inc., Wilmington, Del.).

Methane was used as the sole carbon and energy source for all fermentations. The flow of methane to the fermentor was metered using a Brooks MFX50 series 11 mass flow controller (Brooks Instrument, Hatfield, Pa.). Separate Brooks mass flow controllers were used to regulate the flows of nitrogen and oxygen to the fermentor. In this system the ratios of methane and oxygen to total gas flow could be adjusted to ensure that mass transfer remained in excess. Prior to entering the fermentor, the individual gas flows were mixed and filtered through a 0.2 μm in-line filter (Millipore, Bedford, Mass.) giving a total gas flowrate of 850 mL min⁻¹, (0.52 vol/vol/min) which was held constant for all fermentations. The methane to oxygen ratio was kept constant at 2:1. The oxygen flowrate was varied so as to provide 10% dissolved oxygen in the liquid and a stirrer rate in the range of 1000 RPM. The gas was delivered to the medium 3 cm below the lower Rushton turbine through a perforated pipe. 1.6 liters of a minimal salts medium of the composition given in Tables 6 and 7 were used for the start up of the fermentation. Before inoculating, the fermentor and it contents were sterilized by autoclaving for 1 hr at 121° C. and 15 psig. No antibiotics were used in the M. sp. Tig333-16 fermentation.

Upon inoculation, the fermentr was allowed to proceed as a batch fermentation until the optical density reached 34 optical density units. Samples were taken at 3-4 hr intervals during this time frame to calculate the initial growth rate of the culture. Upon reaching an optical density of 3-4 the feed and effluent pumps were turned on to provide for continuous operation. The feed delivered to the fermenter was split into two fractions, the compositions of which are given in Table 8. The two feed fractions were fed independently to the fermenter at equal flowrates. The pumps were initially started to give a dilution rate of 0.05 hr⁻¹ until the optical density reached a value of ˜20 and no ammonia was detected in the fermenter by ion chromatography (see below). At this point the culture is defined as being ammonia limited. Once ammonia limitation was established the feed rate was increased until the point of ammonia limitation was surpassed and then finely adjusted until the point of ammonia limitation was just reached.

TABLE 8
Feed Composition of Continuous Fermentation Media

| | | Amount |
|---|---|---|
| | Component | (g/L) |
| Feed 1 | (NH₄)₂HPO₄ | 2.25 |
| | (NH₄4)₂SO₄ | 0.5 |
| | NH₄Cl | 3.0 |
| | KCl | 0.6 |
| | NaCl | 0.2 |
| Feed 2 | MgSO₄ | 0.2 |
| | MgCl₂*6H₂O | 0.6 |
| | CaSO₄*2H₂O | 0.3 |
| | CuSO₄*2H₂O | 0.023 |
| | ZnSO₄*7H₂O | 0.0082 |
| | H₃BO₃ | 0.00098 |
| | MnSO₄*H₂O | 0.0018 |
| | CoCl₂*6H₂O | 0.0012 |
| | Na₂MoO₄*2H₂O | 0.00076 |
| | FeSO₄*7H₂O | 0.095 |
| | NiSO₄*6H₂O | 0.0082 |

Ammonia Concentration Determination

10 mL culture samples for ammonia analyses were taken from the fermenter and centrifuged at 10,000×g and 4° C. for 10 min. The supernatant was then filtered through a 0.2 μm syringe filter (Gelman Lab., Ann Arbor, Mich.) and placed at −80° C. until analyzed. Ammonia concentration in the fermentation broth was determined by ion chromatography using a Dionex System 320 Ion Chromatograph (Dionex, Sunnyvale, Calif.) equipped with an AS50 Autosampler, and ED40 Electrochemical Detector operating in conductivity mode with an SRS current of 50 mA. Separation of ammonia was accomplished using a Dionex CS12A column fitted with a Dionex CG12A Guard column. The columns and the chemical detection cell were maintained at 35° C. Isocratic elution conditions were employed using 12 mN H₂SO₄ and 9% acetonitrile as the mobile phase at a flowrate of 1.5 mL/min. The presence of ammonia in the fermentation broth was verified by retention time comparison with an NH₄C1 standard. The concentration of ammonia in the fermentation broth was determined by comparison of area counts with a previously determined NH₄Cl standard calibration curve. When necessary, samples were diluted with de-ionized water so as to be within the bounds of the calibration curve.

### Example 15

### Analysis of Methylomonas Strain Tiq333-16 Continuous Fermentation Production

Growth of Strain Tig333-16 and Production of Carotenoids During Fermentation were Assayed to Assess the Stability of the Strain.

The initial growth rate of strain Tig333-16 was assayed by determining the slope of a semi-log plot of optical density vs. time. The initial growth rate was 0.22 hr⁻¹, which was 38% faster than the growth rate of a carotenoid-producing strain that bears the crtEWYIB genes (Example 8) on a plasmid.

The following plate count assay was used to monitor the stability of the Tiq333-16 strain.

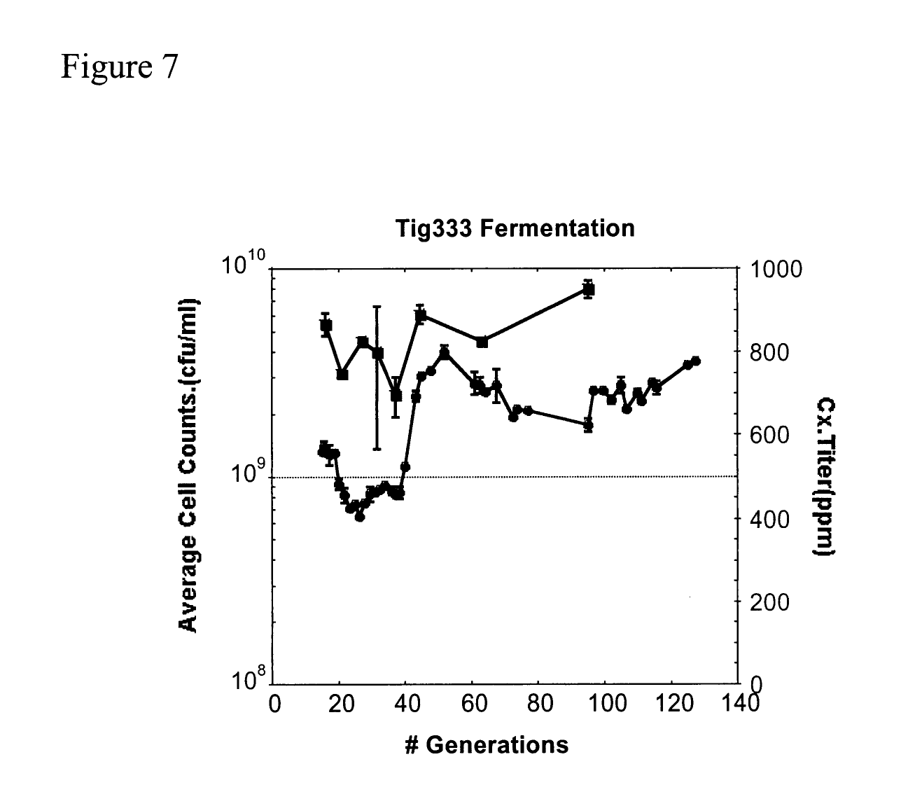

1-mL samples were taken from the fermentor and serially diluted in fermenter medium to give 10^{-b 6} to 10⁻⁷ dilutions. 25-100 μL of final dilutions were plated on fermenter media plates containing 17 g/L agar, incubated for growth, and the number of colonies counted. The results shown in FIG. 7 demonstrated that this strain had maintained complete stability over the 100 generations assayed.

Total Carotenoid Extraction and Identification by High Performance Liquid Chromatography (HPLC)

A 10-mL sample of Methylomonas culture was centrifuged at 10,000×g and 4° C. for 10 minutes in a 50 mL Corning polypropylene disposable centrifuge tube. The supernatant was decanted and the cell pellet frozen at −80° C. The frozen cell pellet was thawed at room temperature and the following added: ˜0.5 mL of 100 μm diameter glass beads, 150 μL ethyl-β-apo-8′carotene(trans) (Sigma Chemical Co., St. Louis, Mo.) (internal standard, 100 mg/L stock solution) and 5 mL 50/50 tetrahydrofuran/methanol (THF/MeOH) solution. The sample was vortexed (Vortex-Genie 2, VWR) for 2 minutes. It was again centrifuged at 10,000×g and 4° C. for 10 min. The supernatant was carefully poured into a new 50-mL Corning disposable polypropylene tube and the cell pellet was resuspended with another 5 mL of 50/50 THF/MeOH solution. The afore mentioned extraction process, without internal standard addition, was repeated 2 more times to maximize canthaxanthin recovery. The supernatants from the 3 extractions were pooled and dried to completion under a stream of N₂. The dry residue was reconstituted in 1.5 mL of 50/50 THF/MeOH solution, filtered through a 0.2 μm Gelman Acrodisc® CR 25 syringe filter into a vial and analyzed by HPLC-MS. The sample filtrate containing the canthaxanthin and intermediates was analyzed using an Agilent 1100 System HPLC (Agilent Technologies Inc., Wilmington, Del.) equipped with a model 1100 Quaternary pump, model 1100 Autosampler, model 1100 Column thermostat, model 1100 Auto sampler, model 1100 Diode-Array detector and model 1100 LC Mass Spectrometer in APCI mode. 20 μL of concentrated extracts were injected onto a 3.5 μm particle size, 4.6×150 mm Zorbax, SB-C18 reverse phase HPLC column (Agilent Technologies Inc.). Peaks were integrated using HP Chem Station software (Agilent Technologies Inc.).

Retention time, spectral comparison in the wavelength range from 220 to 600 nm, and mass to charge ratio (m/z) were used to confirm peak identity with carotenoid standards. Echinenone, m/z 551; 3-hydroxyechinenone, m/z 567; and β-carotene, m/z 537; intermediates in addition to ethyl-β-apo-8′carotene(trans), m/z 460; were identified by their m/z ratio. Canthaxanthin was quantified by comparison of area counts with a previously determined calibration curve as described below. A wavelength of 470 nm, corresponding to the maximum absorbance wavelength of canthaxanthin in 50/50 THF/MeOH, was used for quantitation. A mobile phase consisting of two solvents: 95% Acetonitrile/5% H₂O and 100% THF, Solvent A and Solvent B, respectively was used for reverse phase separation of the carotenoid intermediates. The separation of canthaxanthin was accomplished using a linear gradient elution profile at a flowrate of 1.0 mL/min over 20 minutes. Canthaxanthin calibration curves were prepared from stock solutions by dissolving 1 mg of canthaxanthin (Carotenature, Lupsingen, Switzerland) in 10 mL of 50/50 THF/MeOH. Appropriate dilutions of this stock solution spiked with 150 μL internal standard were made to span the canthaxanthin concentrations encountered in the extracts. Calibration curves constructed in this manner were linear over the concentration range examined.

This analysis showed that production of canthaxanthin was maintained over the 130 generations assayed (FIG. 7). In the plate count assay results of FIG. 7 the only cells detected were those that produced canthaxanthin. This result indicates that Tig333 strain is very stable. At generation 40 the dilution rate was increased from 0.05 hr⁻¹ to 0.135 hr⁻¹, which resulted in an increase in canthaxanthin titer from 450 ppm to around 725 ppm. Although a complete dilution rate profile was not obtained it is anticipated that further increases in dilution rate would yield higher titers of canthaxanthin than measured here.

The plating assay and canthaxnthin analysis indicated that the Tig333-16 strain was completely structurally stable. Integration of the genes for carotenoid production in the tig region of the chromosome resulted in this stability.

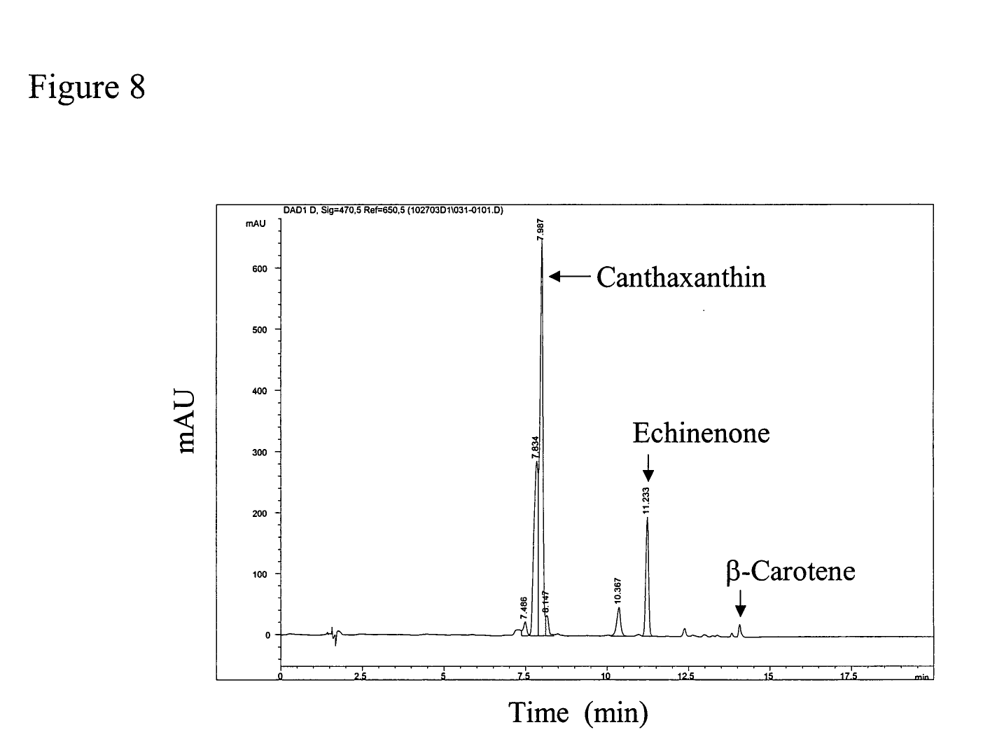

Conversion of β-carotene to canthaxanthin by Methylomonas. sp. Tig333-16 was roughly 80% as determined by normalization of peak areas in the HPLC chromatograms of FIG. 8. FIG. 8 contains an HPLC plot of the uv/visible intermediates of the C₄₀ carotenoid pathway. Of the intermediates to be found, only echinenone and α-carotene were identifiable by comparison of retention time, uv/visible spectrum, and mass to charge ratio. Echinenone appeared to be the major accumulating intermediate.

### Example 16

### Analysis of Canthaxanthin Isomers Produced in Strain Tiq333-16 Fermentation

The isomers of canthaxanthin produced by strain Tig333-16 during fermentation were analyzed to determine whether the preferred E isomers were present.

Extraction and Determination of Canthaxanthin Isomers by HPLC

A 10-mL Methylomonas sample from the fermentor was centrifuged at 10,000×g and 4° C. for 10 minutes in a 50-mL Corning polypropylene disposable tube. The supernatant was decanted and the cell pellet frozen at −80° C. The frozen cell pellet was thawed at room temperature and ˜0.5 mL of 100 μm diameter glass beads (BioSpec Products Inc., Bartlesville, Okla.), 4 mL ethanol, and 6 mL dichloromethane were added. The sample was vortexed for 2 min. and again centrifuged at 10,000×g and 4° C. for 10 min. The supernatant was decanted and saved. Visual observation of the cell pellet revealed that all the canthaxanthin had been removed from the cells. The supernatant was dried under a stream of N₂. The dried sample residue was dissolved in 5 mL of 4.5% chloroform/94.5% n-hexane and filtered through a 0.2 μm Gelman Acrodisc® CR 25 syringe filter and analyzed by HPLC.

The sample filtrate was analyzed using a Beckman System Gold HPLC (Beckman Coulter, Fullerton, Calif.) equipped with a model 125 ternary pump system, model 168 diode array detector, and model 508 autosampler. 20 μL of concentrated cell extracts were injected onto a 250×4.6 mm Brownlee, Sheri-5 Silica-5m normal phase HPLC column (Perkin Elmer, Norwalk, Conn.). Chromatographic peaks were integrated using Beckman Gold software (Beckman Coulter, Fullerton, Calif.). Retention time and spectral comparison confirmed peak identity with all-E canthaxanthin standards (CaroteNature, Lupsingen, Switzerland) in the wavelength range from 220 to 600 nm. A mobile phase consisting of acetone:n-hexane:benzene (2:5:94) was used for normal phase separation of all-E and various Z canthaxanthin isomers. The separation of canthaxanthin was accomplished isocratically at a flowrate of 1.5 mL/min for 20 minutes.

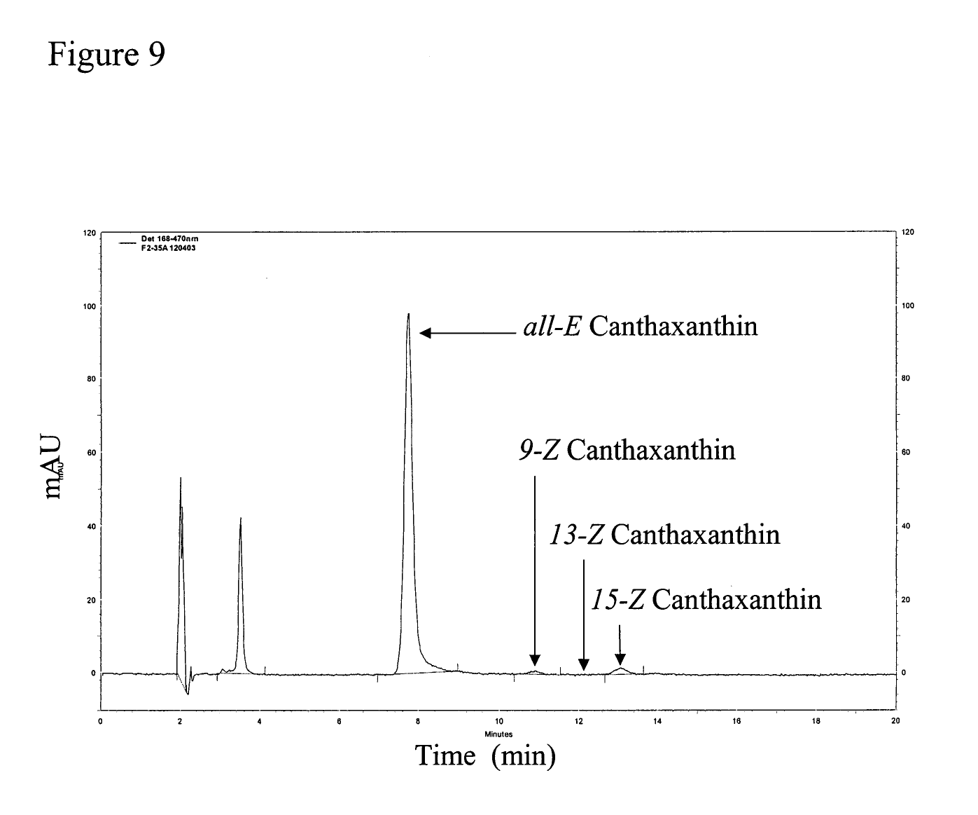

The vast majority of canthaxanthin isomer produced by the Tig333-16 strain was the all-E isomer as shown in FIG. 9. Only minor amounts of 9-Z, 13-Z, and 15-Z isomers were detected. The all-E isomer is required for commercial canthaxathin production for use in salmon feed, as ony the all-E isomer is absorbed and taken up in salmon muscle tissues.

1. A method for stably expressing a nucleic acid molecule in a C1 metabolizing microorganism comprising:
a) providing a C1 metabolizing microorganism having a tig region in the genome;
b) providing at least one nucleic acid molecule to be stably-expressed
c) integrating the at least one nucleic acid molecule of (b) into said tig region of the genome of said C1 metabolizing microorganism; and
d) growing the C1 metabolizing microorganism of c) under conditions whereby the at least one nucleic acid molecule is stably-expressed.

2. The method according to claim 1 wherein the at least one nucleic acid molecule is transcribed using the tig promoter.

3. The method according to claim 1 wherein the at least one nucleic acid molecule is operably integrated.

4. The method according to claim 1 wherein the nucleic acid molecule comprises multiple tandem genes in a single fragment.

5. The method according to claim 1 wherein the at least one nucleic acid molecule is a gene.

6. The method according claim 5 wherein multiple unlinked genes are integrated at different positions within the tig region.

7. The method according to claim 1 wherein the at least one nucleic acid molecule is integrated into the tig region downstream of the tig promoter.

8. The method according to claim 1 wherein the at least one nucleic acid molecule is integrated into the tig region downstream of any gene of the tig region.

9. The method according to claim 1 wherein the at least one nucleic acid molecule is integrated downstream of the tig open reading frame.

10. The method according to claim 1 wherein the at least one nucleic acid molecule is integrated within the Ion open reading frame.

11. The method according to claim 1 wherein the at least one nucleic acid molecule is integrated downstream of the clpP open reading frame.

12. The method according to claim 1 wherein the at least one nucleic acid molecule is integrated downstream of the clpX open reading frame.

13. The method according to claim 1 wherein the at least one nucleic acid molecule is integrated downstream of the himA open reading frame.

14. The method according to claim 1 wherein the tig region is defined according the sequence given in SEQ ID NO:1.

15. The method according to claim 1 wherein the at least one nucleic acid molecule is selected from the group consisting of genes encoding: transaldolase, fructose bisphosphate aldolase, keto deoxy phosphogluconate aldolase, phosphoglucomutase, glucose-6-phosphate isomerase, phosphofructokinase, 6-phosphogluconate dehydratase, 6-phosphogluconate-6-phosphate-1 dehydrogenase, dxs, dxr, ispA, ispD, ispE, ispF, crtE, crtX, crtY, crtI, crtB, crtZ, crtD, crtO, crtW, crtidi, genes encoding limonene synthase, ugp, gumD, wza, espB, espM, waaE, espV, gumH, genes encoding glycosyltransferase genes, aroG, aroB, aroQ, aroE, aroK, 5-enolpyruvylshikimate-3-phosphate synthase, aroC, trpE, trpD, trpC, trpB, pheA, tyrAc, pds, phaC, phaE, efe, pdc, adh, pinene synthase, bornyl synthase, phellandrene synthase, cineole synthase, sabinene synthase, and taxadiene synthase.

16. The method according to claim 1 wherein the at least one nucleic acid molecule encodes at least one enzyme in the carotenoid biosynthetic pathway.

17. The method according to claim 16 wherein the at least one at least one enzyme in the carotenoid biosynthetic pathway is selected from the group consisting of: geranylgeranyl pyrophosphate synthase, zeaxanthin glucosyl transferase; lycopene cyclase, phytoene desaturase, phytoene synthase, β-carotene hydroxylase, β-carotene ketolase and isopentenyl diphosphate isomerase.

18. The method according to claim 1 wherein the C1 metabolizing microorganism is selected from the group consisting of methanotrophs and methylotrophs.

19. The method according to claim 18 wherein C1 metabolizing microorganism is selected from the group consisting of Methylomonas, Methylobacter, Mehtylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, and Pseudomonas.

20. The method according to claim 19 wherein the C1 metabolizing microorganism is Methylomonas 16a.

21. The method according to claim 20 wherein the C1 metabolizing microorganism has the ATCC designation ATCC PTA 2402.

22. A method for the production of a carotenoid compound comprising:
a) providing a C1 metabolizing microorganism comprising a gene cluster comprising genes encoding the carotenoid biosynthetic pathway operably inserted into the tig region of the genome;
b) contacting the C1 metabolizing microorganism of (a) with a C1 carbon substrate selected from the group consisting of methane and/or methanol under conditions where said gene cluster is expressed and at least one carotenoid compound is produced; and
c) optionally recovering said carotenoid compound of (b).

23. The method according to claim 22 wherein the C1 metabolizing microorganism is selected from the group consisting of Methylomonas, Methylobacter, Mehtylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, and Pseudomonas.

24. The method according to claim 23 wherein the C1 metabolizing microorganism has the ATCC designation ATCC PTA 2402.

25. The method according to claim 22 wherein the genes encoding the carotenoid biosynthetic pathway encode at least one enzyme selected from the group consisting of: geranylgeranyl pyrophosphate synthase, zeaxanthin glucosyl transferase; lycopene cyclase, phytoene desaturase, phytoene synthase, β-carotene hydroxylase, β-carotene ketolase and isopentenyl diphosphate isomerase.

26. The method according to claim 22 wherein said carotenoid compound is selected from the group consisting of antheraxanthin, adonixanthin, astaxanthin, canthaxnthin, aanthaxanthin, capsorubrin, alpha-cryptoxanthin alpha-carotene, beta-carotene, epsilon-carotene, echinenone, gamma-carotene, zeta-carotene, alpha-cryptoxanthin, diatoxanthin, 7,8-didehydroastaxanthin, fucoxanthin, fucoxanthinol, isorenieratene, lactucaxanthin, lutein, lycopene, neoxanthin, neurosporene, hydroxyneurosporene, peridinin, phytoene, rhodopin, rhodopin glucoside, siphonaxanthin, spheroidene, spheroidenone, spirilloxanthin, uriolide, uriolide acetate, violaxanthin, zeaxanthin-β-diglucoside, zeaxanthin, and canthaxanthin.

27. A C1 metabolizing microorganism comprising at least one nucleic acid molecule integrated in the tig region of the genome.

28. The C1 metabolizing microorganism according to claim 27 wherein the at least one nucleic acid molecule lacks an antibiotic selection marker.

29. A method for identifying an integration site in a genome for high level expression of a nucleic acid molecule in a microorganism comprising:
a) providing a microorganism;
b) providing an integration vector comprising a gene cluster encoding at least the following enzymes geranylgeranyl pyrophosphate synthase, zeaxanthin glucosyl transferase; lycopene cyclase, phytoene desaturase, phytoene synthase, β-carotene hydroxylase, β-carotene ketolase and isopentenyl diphosphate isomerase, wherein said integration vector is designed to facilitate the integration of the gene cluster in to the genome of the microorganism;
c) contacting the integration vector of (b) with the microorganism of (a) under conditions which allow for random integration of the gene cluster into the microorganism genome to create random tranformants;
d) screening the random transformants for expression of the gene cluster on the basis of the production of a C₄₀ carotenoid; and
e) identifying sites of integration of the gene cluster into the genome of the random transformants.


SEQUENCE LISTING (60 sequences)

SEQ ID NO: 1 - DNA, 9010 bp, Methylomonas sp. 16a
caggcccagggcgcgtagcgcttcggtgtcgacgatttcgatatcgatgcggtgattattggcgaacgtcgtgctcccccattgcgtgaggtcttggctcgaagcggattgcacgctcaagcttgtcattaggacgaggaatccgcaaaaaatggccgtcagtggagtgttcatgtcagggaagattgatttttggttgtttttgtggggcggccttattataatgccgcgttcatttatattgcgagcgtggcgaaattggtagacgcgctggatttaggttccagtggtaaccccgtgagagttcgagtctctccgctcgcaccatttctttctgttattcgcagcgatccagcatcttttcctgagtcatgaatcccgctgatcgaccagtctcccaattctttctttttaacttttgaggtaaaccaatgcaagtttctgtcgagaagacatcagaattaagccgaaaaatgactgttagtgtgcctgatgcggtgcttcaggaaaaaatggaaactcgctttaaaaaactggctcgagaagttaaggttgatggttttcgtcccggtaaagtgccagtcagcacggttaaaaagctttatggcgagcgcgtcaaacacgaagtggccggcgatctgattcaatcgacttatttcgaagcgttgcagcaacaagaattggtgcctgccggccatccgctgatcacgcccgcggacaaaagcgaaggttttgagtatgtggctgaattcgaagtttaccctgaaatcggtctggactcggtcaatggcctggaaatcagtcgtccagtcgctagtgtgaccgacgctgatgtcgaaaacatgatcgaaaagttgaggcagcaaaagaaaacatggcaggtcgtcgaacgggcctcacaagaaggcgacaaagtcaccattcatttctcaggtgtgtctgaaggcgaaaatttcaccgacggcaaggtggaaaattacgcgatcgagatcggcggcaagcagatgattcccggctttgaagacgagctgaaaggcttggccgccggtgaacgcaaaagcttcaacatcacttttccggaaaaatacaacagcgaaaaattggccggcaaagccgctgatttcgagatcgagatgatcaaggtcgaagagccggtattgcctgaactggatgccgacttcatcaaggcctatggtgtagaagcgggcgatgtcgtcagttttcgcgccgacgtcaaggaaaacatggaaagagagttggcgcaaggtctgaaaaacaagctcaaaaccgccgtgttggatgcgctgtacgaaaacgtcaagatcacgctgcccaacgcgttgatcgatcaagaagttcaagcgttgatgaagccttatgcggaacgtgccggcaaggccaagttgaaattggaagatttgaatttgccgcgcgatgtcttcgaagaacaagccaagcgccgcgtggcattgggtttgattttgggcgaaatcatccaaaagaacgacatcaaggtggatgccgataaagtgcgtgccgtgattgacgacatggcgaaaagttacgaaaagccagaagacgttgtaaattggtattatgccgacaatagccgcttaaacgacgttcagcaaatggtgctggaagatcaggccgtggactgggttgtcgagcgtgctcgggttaccgaccaaaatgtcggttttaacgatgtcatggaacggcagcaagcataggaaactgaatgattgatctaactggcatgaatactatggttccccaggccgcgggcggtctggtgcccatcgtggtggagcaaacggcgcgcggcgagcggtcgtttgatatttattcccgcttgttgaaagagcgggttatttttctggtgggacaggtcgaagactacatggccaacctggtcgtggctcaattgctgtttctggagtcggaaaatccggacaaggatatacacctgtatatcaattcgcccggcggttccgtgacggcaggcatgtcgatctatgacaccatgcaattcatcaagcccgatgtcagtaccatgtgcatcggtcaggcggccagtatgggggccttgttgttggcgggtggcgcggctggcaagcgttattgcctgccgcattcacgtgtgatgatccatcaacccttgggcggtttccaggggcaggcatcggacatcgccattcatgccaaggaaattttgtccatccgcgataagttgaacaagattctggcgcatcatacggggcagccgttggaaaaaatccagcaagacaccgatagggataattttttgagttccgaacaagcggtcgaatatggtctgatcgataaagttctgagcagccgtaacgtttaagccaaactcgccgtaatgcggcgagttaattgggaaagttgatatttttgatatattcttcagccactgaagcaatgtattaccccggggtctgttcatgagtagagacaaaaaaggtaaagacgaagataaacttctttactgttctttttgcggcaaaagccaaaacgaagttagaaagctgattgccgggccgtccgtgtatgtctgcgacgaatgcgtggagctttgtaacgacatcattcgtgacgaattggccgaagacgaaaaaagcgtcggcagcggtgccttgccgaaaccaaaagaaatcaagcaagagctggataattacgtcattggtcaggaaaaggccaagaaaattctatccgtcgcggtttacaaccactataagcggttacgcagcaacagcaaaaaaagcgatgtcgagctggcgaaaagcaacattctgttgatcggcccgaccggctccggtaagacgctgttggcggaaacgctggcacgcttgttggacgtgcctttcacgatcgccgacgccacgacgctgacggaagccggttatgtcggtgaggacgtcgagaatatcattcagaaaattctgcaaaagtgcgattacgatgtcgagaaagcggaaaccggtattgtctacatcgatgaaatcgacaaaatttcacgcaaggccgacaatccatccatcacgcgcgatgtgtccggcgaaggcgtgcagcaagccttgctgaagctgatcgaaggcacggtggcttcagtgccgccgcaaggtggacgcaagcatccgcagcaggaattcttgcaggtgaataccgccaatattctgttcatcgtcggtggcgcttttgccggcttggataaagtcatcaaaaatcgtaccgaaaaaggcggcattggtttttcggcggagctcaaaccgaaggaggagagtcaaaatgtcggtgagatttttgccgatgtgcgcgccgaagacttgatcaaatacggattgatcccggagtttgtcggtcgattgccggtggtggccacgctggacgaattggatgaacacgccttggtgcaaatcctgacccagcctaaaaacgccctgatcaaacaatacaggcatttgttcgaaatggaaggggtggaactggagttcagagaagactccttggcggcgattgcgcgcaaatccatggaaagaaaaaccggcgcgcgtggattgcgcaccatcgtcgaaaacgtgctgctggataccatgtacgaactgccttccagcgacagcatcaccaaggtcgtggtcgacgaaagtgtcatcaatggtcaatccgagccgattctggtgtatgagccggaattgaagcgtgcgtcatccgattgaagctttcggcgctgatgcgtgacaaggaaaggggctttaccgcaaagccccttttttttgccgaatggacgaagatacttgttttctctgcgatcgccctcatatacgattgcagctaaattgctttcctgccaaaggacacgaatcaaatgactcaagtaaaaaataaagacgatcttattcctgtgttgccgcttcgggatgtggtggtatatccgcatatggtgatcccgctgttcgtcggccggggaatgtcgatcgatgcgctcgatgccgccatcaaacaggacaaacaagtgctgttgatcgcgcaaaaacaggcggatatcgacgagcccgagtttgacgatttgtataaggtagggacgctggccaatattctgcagttgctcaagttgccggatggcaccgtcaaggtgttggtcgagggaactcagcgttgctcggtcgtgcaatatcaagaatttgaaggctattgcgcggcgggcgtactcgagctggaagatcaaatcacgatcgacgagcaagaactcgacgtattacaacgcacggcgatcaactcgttcgatcaatatgtcaaattgaacaataagattccgccagaagttctgaattctctgtccggtatcgatgatcccggtcgcctggccgacaccatggcggcgcatatggcgctgaaggtggaagaaaaacaaaaaatgctggagatggtcgatgtcgcccggcgcctggaaaatttgatgacgctgatggagggcgaagtcgatatcctggaaatggagaaaaagattcgcggccgggtcaagaaacaaatggaaaaaaaccagcgcgagtattatctgaacgagcaaatgaaggcgattcagaaagagctgggcgagatggacgaagccagcaacgaagtcgaagagctggaaaagaaaatcgaggcggccggcatgtcggccgaagccaaaagcaaagccatggccgagctgaacaagctgaaaatgatgtcgccgatgtcggccgaagcgaccgtcgtgcgcaactatatcgattggatggtcagcgtgccatggaagaaaaaaaccaaggtacgccatgatctcaaggtcgccgaacaggtgctggaatccgaacattacggtctggaaaaggtgaaggaaaggattctggaataccttgcggtacaacaacgggtgaaacaattgaaagggcctattctgtgcctggttgggccgccgggcgtgggtaaaacctcgttgggccagtccatcgcgcgggcgacgaatcgcaaatacgtgcgcatggccttgggcggcgtgcgcgacgaagccgaaatccgcgggcacaggcggacgtatatcggttcgatgccaggcaagattttgcagaatctcgccaagatcaaaacgcgcaatccgatgttcctgctggacgaaatcgacaagatggcggcggattttcgcggcgatccggcgtcggccttgctggaggtgctggatcccgagcaaaatcacaccttcgccgatcattatctggaagtggatttcgatttgtccgacgtgatgttcgtggcaacggcgaataccctgaatattccggcgccgctgatggacagaatggaggtcattcgtctcgcaggttataccgaagacgaaaaaatcaatatcgccacgcgctttttgattcccaagcagatcaaaaacaatggtttggccgaagccgagattgaaattacggaggctgccgtcagggatatgatccgttattacacgcgcgaagccggcgtgcgtagtttggaacgggaaatatcgaagatctgccgcaaagtggtgaaggatttattgctgcataacggtaaagaaaaaatttgtgtcgatagcaaaaacctggataaatatctcggcgtgcgtcgcttcagctatggcatggccgaggatagcgatcagatcggtcaagtgaccggcttggcttggaccgaagtgggcggtgaattgctgactatcgagacggccgtcatgccgggcaaaggcaagcagcaagccaccggtaaactgggcgacgtgatgaaggaatccatcgatgccgccatgacggtggtcaggagtcgtgcggaaatactgggcatagacaaggaggttttccagggtagcgacattcacgttcacgtgccggaaggcgcaacaccgaaggatggtccaagcgcgggcatcgggatgtgtacagcgattatttcggcgctgaccaaaattccggtcagggccgatgtggcgatgaccggcgagattacgctgcgcggtgaggtattgccgattggcggtttgaaagagaagctgttggccgcgcatcgcggcggcatcaggaccgtggtgattccatccgaaaacgaaaaagacttggctgaaatacccgataacgtcaaaagcaatttgaccatcaaatgcgtgcgctggatagacgaggtgttagagatcgcgctgcaaagaatccctgttccgattgcggccgcggaaggtcaaaccgccgaggggcccaatggcaaaaacgaagcgaaaccgcaggcggtgctcgcgcattaattttgaagtcgattcgctgctaaaccatgaaattcgcggcctgcggcgattacaacgcttgacatggctcgtggcccattgatataaagtcccaccttcttgatatgaaggtggctatgaatgggagggggcgagcctgttggtgccgaaatcaaatcgcttgtttgcttgagcataattaatacaacgacatcctaaggggaaataaatgaataaatcggaactgatcgatgctattgctgcagcatctcatttgacaaaagccgatgccggccgtgctttagatggttttatcaagtctgttgaagaagcgctgaaaaaaggcgattcagtagccttggttggttttggcaccttcgaagttaaagaaagagcggaacgcaaaggccgtaatccgcaaaccggcgaagaaatcactatcaaggccgcaaaaattccttcatttaaagctggcaaatctttaaaagatgcggtaaactagtcgctctttagtcgggtgcttagctcagctgggagagcatcgcccttacaaggcgagggtcgggggttcgaacccctcagcacccaccagacttaggagcggtagttcagttggttagaataccggcctgtcacgccgggggtcgcgggttcgagccccgtccgctccgccaaaacagaaacctcgggccgtacggttcgaggtttttttttgcgtgttcatkttsycscctgtttggtcaggttaaacgctgtttaaggacagagctatgctattagagattagagaaaaggtgcagggtgtatttgcatcgatcattttagtgctgatttgtgtgctttttggcttatggggtatccagaattatttgggcggcggtaaggaggcccccgtcgttaccgtgggcgataaggatttttttcagcgcgacgttactcaggcctatcagcagtttgctcagaacctggcaggcatgaaattcgacgaagaaacgctgaaaaagcaagccgtgcaaaaactgatacgcgatgaagtgctgttgcaatatgcgcaagaacagaatttggttatcacggacgaaaccgctagaaatttcattcaatcactggaatatttccaaaaagacggccagttcgataagggccagtatcaagcgttgctgagttcgcaaggcatgtcttccgatgatttcgtcaatcgggtgaaaaaggcgttgatgatggaacaggtgcagcatgccgtggtcgagagcggtttcgtgaccaaggctgaaatcgaaagctttttcaagctccaaaatcaaacccgggatgtcgagtatctgacagtacccctggcctcatccagcgaattgccggccgatgacgagataaatgcctattatcaacaacatcaagaggcttaccaaaccccggagcgggccgtcatcagctatgtggaattatccttggatgatttggctaaggatgtcagcgcgtccgatgagcaattgaaagcctattacgaagagcaaaaggcgcaatacactacgcccgaaagacgcaagatcagtcacatcctgtttgcgtttggcaaggacagtgccgatgatcaacaagccttgcaaagagcattgaaggccaagcaagatttggcaaacaaggattttgccgcgttggccgcggaagtatccgatgacaaactgaccgcgaagaatggcggcgatctgggtttgttcaatgtcggcgtgatggaaaaatcctttgaagaggccgctagccagttgaagcaaggcgaagtttcggagccggtcaaatccgcctttggttatcatttgatcaaggtgaccgaattggcgccgggcgagatcaagccttacgaggcggtaaaatcggaattgaccaaggcgtataaaaaaggccaaatcgaaagtcgctttagtgagctgggagaaaaactcagcgaagtgagttacgaaaatccggataatctggatgccgcagcgaagttgcttggggtgccggtcaagacatcggcgccgtttacccgtgatgccggtgaaggtgttgcggtcgatgaaaaagtccgcttggcggctttttccgaagatgtgttgaagggtaataatagcgagccgatcgagattggcagcgagaaattggtggttttgcgcatgcaatctcatacgcctgctgccgccaaggatctcaaggaagtcaaggcggacgtgattgctgcgatacaaaaagagaaggcgctgaagcaagtcgtggaaaaggccgataagctcaaggctgaattggttgccggtaagccgattgcggatgtggcttccgcgactgctttgacggtcaagaaagtggttggcttgagtcgcaatgctggcgatgtggacccggccgtcaggcaggcaattttccgggcggccaagccgcgggcaaatcgtccgagtatcgtggtgatcgatgagcctgctggcggtaaaattgtcgccagtatctctcaagtcaaggaaggtgtcatgactgaggccgacaaggccaagcaagtgcagctggagaaaaatatggcgaccgcatttggcaggacgcaattcgaagccgtgctgaaccagttgcaagccaacgccgatattacgatacgatctcccaagcaataaccgatacccatcatgaccacgatccgccggatcgtggtcattcccctgagctttgcaacaggtaaatctgtcccagcttccactc

SEQ ID NO: 2 - DNA, 1536 bp, Methylomonas sp. 16A
atggccaacaccaaacacatcatcatcgtcggcgcgggtcccggcggactttgcgccggcatgttgctgagccagcgcggcttcaaggtatcgattttcgacaaacatgcagaaatcggcggccgcaaccgcccgatcaacatgaacggctttaccttcgataccggtccgacattcttgttgatgaaaggcgtgctggacgaaatgttcgaactgtgcgagcgccgtagcgaggattatctggaattcctgccgctaagcccgatgtaccgcctgctgtacgacgaccgcgacatcttcgtctattccgaccgcgagaacatgcgcgccgaattgcaacgggtattcgacgaaggcacggacggctacgaacagttcatggaacaggaacgcaaacgcttcaacgcgctgtatccctgcatcacccgcgattattccagcctgaaatcctttttgtcgctggacttgatcaaggccctgccgtggctggcttttccgaaaagcgtgttcaataatctcggccagtatttcaaccaggaaaaaatgcgcctggccttttgctttcagtccaagtatctgggcatgtcgccgtgggaatgcccggcactgtttacgatgctgccctatctggagcacgaatacggcatttatcacgtcaaaggcggcctgaaccgcatcgcggcggcgatggcgcaagtgatcgcggaaaacggcggcgaaattcacttgaacagcgaaatcgagtcgctgatcatcgaaaacggcgctgccaagggcgtcaaattacaacatggcgcggagctgcgcggcgacgaagtcatcatcaacgcggattttgcccacgcgatgacgcatctggtcaaaccgggcgtcttgaaaaaatacaccccggaaaacctgaagcagcgcgagtattcctgttcgaccttcatgctgtatctgggtttggacaagatttacgatctgccgcaccataccatcgtgtttgccaaggattacaccaccaatatccgcaacattttcgacaacaaaaccctgacggacgatttttcgttttacgtgcaaaacgccagcgccagcgacgacagcctagcgccagccggcaaatcggcgctgtacgtgctggtgccgatgcccaacaacgacagcggcctggactggcaggcgcattgccaaaacgtgcgcgaacaggtgttggacacgctgggcgcgcgactgggattgagcgacatcagagcccatatcgaatgcgaaaaaatcatcacgccgcaaacctgggaaacggacgaacacgtttacaagggcgccactttcagtttgtcgcacaagttcagccaaatgctgtactggcggccgcacaaccgtttcgaggaactggccaattgctatctggtcggcggcggcacgcatcccggtagcggtttgccgaccatctacgaatcggcgcggatttcggccaagctgatttcccagaaacatcgggtgaggttcaaggacatagcacacagcgcctggctgaaaaaagccaaagcctga

SEQ ID NO: 3 - DNA, 1593 bp, Methylomonas sp. 16A
atgacgacgatagcagccgtctccccactggatggccgcttgctgggacattttccagtcagcaagccggcgctcattcagcaacagctgacaaaatcccgccgcgccgccctgctttggcgcgagctgccggtcacggaacgggtcaaacgcctgtcgcccttgaaaaaacagctgctggataacctggacagactctgcgaaaccatccgcctcagcaccggcaaggttcgcaccgaggccttgctgggggaaatttatccggtgctggatttactggcgtattaccaaaagcgggcgccgcggattctacgcacgcgcgccgtgtccacctcgccgttcgcgtttccggccgccaccgcccgcatcgaacgccgcccttacggcgtggtcgcggtgatctcgccatggaattacccgtttcacctgagcgtcgccccgctgctgaccgctttgctggccggcaatgcggtaatcctgaaaccctccgaactctgcttgccggtcggtcagttgatcgtcgatttgttcgccacgctggatttgccggacgggttggtgcaatgggtcatcggcgacggccaaaccggcgcggaactgatagacgcccgccccgatctggtgtttttcaccggcggcctgcagaccggtcgggcggtcatgcaacgcgccgcccggcatccgattccggtcatgctggagttgggcggtaaagacaccatgctggtgctggccgacgccgacctcaagcgcgccagcgctgccgcgctgtacggcgcgttttgcaatagcggccaagtctgcgtctcggtcgaacgtctgtacgtgcaacaagcctgttttgcggaattcctggccatgctgctgaagggcctgtccaagctcaaggtcggccatgacccgcacggcgatgtgggagtgatgacgtccgcccggcaaatcgacatcgtccaggcccattacgaggacgccatcgcccagggcgccaaggcctccggcccgctgctgcgcgacggcaatgtcgtgcaacccgtggtgctttgggacgtgcaccacggcatgaaggtcatgcgcgaggaaaccttcggtccgttgctgccggtcatgccgttcagcgacgaagccgaggccatcaagctcgccaacgacagcgatctgggtctaaacgccagcatctggagccaggatataatcaaggccgagcgccttgctggacaactagatgtcggcaactgggcgatcaacgacgtattgaaaaacgtgggccattccggcctgcccttcggcggcgtcaagcaaagcgggtttggccgttatcacggcgccgaaggcttgctgaacttcagctacccggtatcgggcctgaccaatcgcagccgcttgcccaaagaacccaactggttcccttacagcgcatcaggctatgaaaatttcaagggtttcctcgattttatctacggcgaagactcgatgctgcagcgcggtcgccgcaatcagcaagcgctgcaagccttccgcgagttttccattttcgattggacacaacgctggcaaaacctgaaactgctgttttcttggacacgggatgactaa

SEQ ID NO: 4 - DNA, 1494 bp, Methylomonas sp. 16A
atgaactcaaatgacaaccaacgcgtgatcgtgatcggcgccggcctcggcggcctgtccgccgctatttcgctggccacggccggcttttccgtgcaactcatcgaaaaaaacgacaaggtcggcggcaagctcaacatcatgaccaaagacggctttaccttcgatctggggccgtccattttgacgatgccgcacatctttgaggccttgttcacaggggccggcaaaaacatggccgattacgtgcaaatccagaaagtcgaaccgcactggcgcaatttcttcgaggacggtagcgtgatcgacttgtgcgaagacgccgaaacccagcgccgcgagctggataaacttggccccggcacttacgcgcaattccagcgctttctggactattcgaaaaacctctgcacggaaaccgaagccggttacttcgccaagggcctggacggcttttgggatttactcaagttttacggcccgctccgcagcctgctgagtttcgacgtcttccgcagcatggaccagggcgtgcgccgctttatttccgatcccaagttggtcgaaatcctgaattacttcatcaaatacgtcggctcctcgccttacgatgcgcccgccttgatgaacctgctgccttacattcaatatcattacggcctgtggtacgtgaaaggcggcatgtatggcatggcgcaggccatggaaaaactggccgtggaattgggcgtcgagattcgtttagatgccgaggtgtcggaaatccaaaaacaggacggcagagcctgcgccgtaaagttggcgaacggcgacgtgctgccggccgacatcgtggtgtcgaacatggaagtgattccggcgatggaaaaactgctgcgcagcccggccagcgaactgaaaaaaatgcagcgcttcgagcctagctgttccggcctggtgctgcacttgggcgtggacaggctgtatccgcaactggcgcaccacaatttcttttattccgatcatccgcgcgaacatttcgatgcggtattcaaaagccatcgcctgtcggacgatccgaccatttatctggtcgcgccgtgcaagaccgaccccgcccaggcgccggccggctgcgagatcatcaaaatcctgccccatatcccgcacctcgaccccgacaaactgctgaccgccgaggattattcagccttgcgcgagcgggtgctggtcaaactcgaacgcatgggcctgacggatttacgccaacacatcgtgaccgaagaatactggacgccgctggatattcaggccaaatattattcaaaccagggctcgatttacggcgtggtcgccgaccgcttcaaaaacctgggtttcaaggcacctcaacgcagcagcgaattatccaatctgtatttcgtcggcggcagcgtcaatcccggcggcggcatgccgatggtgacgctgtccgggcaattggtgagggacaagattgtggcggatttgcaataa

SEQ ID NO: 5 - DNA, 1542 bp, Methylomonas sp. 16A
atgcgcggcattcatccagaatccaaacacgaacaatacgacgtgatcgtcataggcgccggtatcggcggcttgagcacggccgcgttattggcgaaagccggaaaagccgtgttgttagtcgaaaggcatgaccgccccggcggttatgctcacggtttccggcggcgcaattaccatttcgattcgggggtacatctggtcagcggctgcggtgccgacggctatgaaaacggcagcacgatttaccggatttgccgggccgtgggcatagaccccgaggatgttttccttccgatcccgtcttacgcccgcgcggtgtttccggggttcgaactgagcctgcatgccggcgaagaggtgttcgtcggtgagttatgcgcgcatttcccaaacgaaaaggacaatctgctccgcttgattcggctctgcaaaaccctggcggaagaagccatgctggcggaagaaattctggaacagagcaaaatcactcgcgtaccacccacgcgagcgctggccaatttgtttcgttaccgccgcgccaccttggcggaagcactggatgaatttttgctcgacccacatcttaaaagtgcctgcgccgcgctatggccttatttgggcctaccgccttcgcaactgtctttcttatattgggccagcatgatggcgggctacacctacgaaggtgcgtattattgccgcggcagttttcaaacctatgccaacagactggcgcaagcgatcgaaaagcgcggcggcgaggtgttattgaacgccagcgtgcggcggatttgcgtggaaaacggcggcatcagcggcatcatgctggaaaatggtcaactaatacgcgcaaagaccgtagtctcgaatgtcgccgcccagcaaaccgccgaattactgatcggtcgcgagcattggccggctggctattgcgacaagctggaaaagttggcgccgtcgctgtcgattttcgccagctacatcgcaaccgatttggccatcgacacggccgttcatagccacgagtcgtttttttaccaaaccttcgatcacgaagccgggtttgcatccacgcacaaggggcagcccaattggttttcggccaccctgtcgacgttgagcgatgcctcgctggcaccggccggtcaacacaccctgatgctgaccaccttatgcccgtttgacatagggcaaagctggcgacaggccaaactggactttgagcaacgcttattggcgcaagccgaacaacattttccaggcctgaaagaccatttgttgctgatagaatccggctcgccgcgcacgctggaacgctacaccctcaaccaccaaggcgcggcctacggctttgcccctacccccgatcaaatcggcccaaaccgtccggacgttcgcggagccttgccgggcttgttccacaccggccactggacgcgtccgggcggcggcgtcgccggcgtcagtatctcggctcaactggcggcacaagccattttgaacctgcccatacaagccgatttctggaacagcctggat

SEQ ID NO: 6 - DNA, 5632 bp, Pantoea agglomerans
catgaccggcggcgcggcgcgcgccagagacattaaccgtcatctggcccaggcggcgcaaacccttgggctggcgatgggcgtcggttcccagcgcgtggcgctggaggacggcgcgcagcacgggctggatgcccagctacgccatatcgccccggacgtgccgctgctggctaaccttggcgcggcgcagatccgcggtgcgcaggggctggactacgcccggcgcgcggtggacatgatcgacgccgacgcgttaattgtgcatctgaacccgctgcaggaggcgctccagggcggcggcgatcgcgactggcgcggcatcctcaacgccattgcgcagctggtgcgcgacctgccggtaccggtggtggttaaagaggtgggcgccgggatctccccggacgttgcctgccgactggcggacgtcggcgtggcgatgatcgacattgccggcgcgggcggaaccagctgggcggcggtggaagctgaacgcgccccgacccccgaggcgcgaaatgtggcgatggcctttgccgactggggcattcctactgccgatgcgctgcgtcgcgtccatcttgcgctgcctgatatcccgcttatcgcctccggcggcatcgccaacggcattgacgcagcaaaagccatcgcgctgggtgcagatctggtgggccaggccgcggcggtgctggcgcatgccaacgcctccggcgacgcggcaattgcccatttccgcaccctgattacgcagctgcggatcgcctgtttctgtaccggcagtgcaaacctgcaggcgttgcgacacgccacgctgcttccggtcaacggcggcgcatccctgtgacgcagtacggtgccttataccggggagcggtatgaaaaaatgggatctgattctggtcggcgcggggctggccaacgggcttatcgcctggcgactaaagcagcgtcatccgacgcttgctgtattaatgctggagtgcggcgacgcgcccggcggaaaccacacctggtcctttcaccaacacgatatcacgccagcccagcacgcctggctggcgccgctggtggcccatcgctgggacgggtacgacgtccactttccgaacgtgtcgcgcaccctgcatgacggctacctgaccatcacctccacgcgttttgcccaagcgatgcgcgggctgatgaaagagaatttgctgacaaacgtgaccgtgtcacgggtgagcgggcaggaagtaaccctcagcgacggacgacgctttaccgccggggcggtgattgatggccgcggctatcagccctcgccgcacctcagcattggctatcaggcgttcatcggccaggagtggcaactgaccgcgccccacgggttaacgcgcccgatcctgatggatgcccgcgtcgcccagggcaacggctaccgctttgtctataccctgccgctcagcgccgacaccctgcttatcgaagacacgcactacattgacggcccgacgctcgacgccgattcagcccgcgcgcggattgccgattacgcccgccagcagggctggcagcttgcgcggctggtgcgtgaggaacagggggcgctgccgatcaccctgtccggcgatccggccgccttctggcaccagttccatcatcagccggtcagcggcctgcgcgccggtctgttccatgccaccaccggctattcgctgccgctggcggttcggctggcggaccgcattgccaacgcgccgggactgcatcagggcgcgctctatcagctgatcgccgatttcgcggcgcgccactggcagacacaacgctttttccgcctgcttaaccgcatgcttttcctggccggcacacccgaccagcgctggcgcgtgatgcagcggttttaccagcttgacgagcagctgatcgcccgtttttatgccggccagcttcgctccgccgaccgcgcgcgcctgctgcttggcaaaccgccggtgccgattgtcggggcgatcaaagccctgctccacactcattcttctctgcgagcccatcataaatgaaacaaaccattgtaattggcgccgggttcggcggactggcgctggcgattcgcctccaggcggcgggcattcctaccacgctgctggagagccgcgacaaacccggcggccgcgcctatgtctacgaagatcgcggctttacctttgatgcgggtcccaccgtcatcaccgatccctccgccattgaggagctgttcaccctcgccggaaaacggctgaaggactacgttgagctgatgccggtgacgccgttctatcgcctgtgctgggaagacggcaaggttttcgactacgccaacgatcaggcggcgcttgagtcgcagatcgccgcgtttaacccgaacgacgtggcgggctatcaccgcttcctcgactactcccgggcggtgtttgccgaaggctatctgaagctcggcgcggtgccgtttctctcgtttcgcgacatgctgcgcgccggtcctcaactggcgcggctgcaggcatggcgcagcgtgtacgacaaagtgtcggcctacgtggaagacgagcacctgcggcaggcattttcgtttcactcgctgctggtgggcggcaacccgttctccacgtcttctatttacaccctgatccacgccctggagcgggaatggggcgtctggttcccgcgcggcggcaccggtgcgctggttcagggcatggtgaagctgtttcaggatcttggcggcaccctcacccttaacgctcaggttgagcggctggagacggtggacaatcaggtgaaggccgtgcatctggttaacgggcagcggctggaggctgcggcggtggcctcgaacgcggacgtggtaaatacctatgcccgactgctcggccatcacccgcacggcgccgctacggccaaaaagctgaaacgcaagcgcatgagcaactcgctgttcgtgctctattttggcctggatcaccatcacacccagctggcgcaccataccgtctgctttggcccgcgttataaagcgctaatcgatgaaattttcagcgccgacaccctgtcggaagatttttcgctctatctgcatgcgccctgcgtaaccgacccgtcgctggccccgccggggtgcggcagctactatgtgctcgcgccggtgccgcacctcggtaacgccccgctcgactggagcgtggaagggccgcgtctgcgggatcgcatttttgattatctcgaagcgcgctatatgccggggctgcgctcccagctggtgacgcaccgcatgttcacgccggaagattttcgcgatacgctcgatgcctggcaggggtcagcgttttcactggagccgatcctcacccagagcgcctggttccggccgcacaaccgcgacagcgtggttgataacctctacctggtcggcgccggaacgcatcccggcgctggcgtgccgggcgtgatcggatccgccaaggcaacggcccagttaatgttaaaggatttagcgtaatgtcccagccgcttctcgaacacgccagcgccaccatgaccgccggttctaaaagtttcgccaccgcctcaaagctgtttgacaaacgcacccggcgcagcgcgctgatgctctatacctggtgccgctactgcgacgatgttatcgacggacaggtggtgggttttgctgccccgaccgagcagagcgacacgcccgaggcgcgcctgcaacggctgcgtaagatgacgcgccgcgcctacgacggggaaaccatgcaagagccgccgttcgccgcctttcaggaggttgccctcgcccatgccattccgcctactcaggccttcgaccacctggaaggctatgcgatggacgtgcgcaacgagcgctattacagcctcgatgatacgctccgctactgttatcacgtggcgggcgtggtcggcctgatgatggccagggtgatgggagtgcgggacgaagccacgctggatcgcgcctgcgatctgggcattgcctttcagctcaccaatatcgccagggatatcgttgacgatgcgcaggtgggacgctgctacctgccgcagcagtggctggcggaagtcggactcaatgaacagacctgcaccgtgcgggccaaccgtccggcgctggcgcgtctggcagcgcggctggtgaccgaggctgagccctattatcagtcagcgcttgccgggctgggggatctgcccctgcgctccgcctgggcgattgccaccgcgcacggggtgtatcgtgagatcggggtgaaggtgctgatggcgggtgaaaaagcatgggatacccgccagggcacgacgcgcgcggagaagctggcgctggttatttccggcgcgaagcaggcgatggcttcccggaaggcgagctggccgccgcgcgatccgcacctctggcagcgcccgcgctagaattcgaattcactagtcgagacgccgggtaccaaccatgacaagaccctttgaaacacatcccggtcacgacggggaactgcatgagctgcacgctgccctgcaacgtcgcctggatgaactgctgcccgttggcgatgagcgggatcgggtcagcagcgcaatgcgcgaaggcgtactggcaccggggaaacgcattcgcccgctgctcctgatcctcgccgcccgcgacctcggctgcgatcgcgaccaccccggcctgctggatatggcctgtgcggtggaaatggtgcacgcctcgtcgctgatcctcgacgatattccctgcatggataacgcggcgctccggcgcggtcgccctaccattcatcgccagtatggtgaagacgtggcaattctcgctgcggtagcgttgctcagcagcgcctttggcgtgatggtcgcggcgcagggattgtctcccgagtgccgcagccaggcggtggcggagctgtcgatggcggtcggtacccagggtctggtgcagggtcagtataaggatctgcgtgaaggcaccgccccgcgcagcgccgaggagatcgccaccaccaacgaactgaaaaccagcgtgctgtttggtgccacgctgcaaatcgcggccctggcggcaggcgcctcgccggcggcgcgccagaaaatgcgctgctttgcgcaggatttaggccaggcgttccagctgctggacgatctggcggacggccatgccgggaccggcaaagacatcaataaggacgcgggtaagtccacgctggtggcgatgctcggcagcgacgcggtgcgcgagcggctcgacacccatctgcgccgcgcagacgcccatttttcacgcgcctgcggaaaaaaccaggccacgcgacgctttatgcacgcctggttttcaaaacagctggccgcgtttagctgagcaacggatacaccccggtaatatttgtggagatcacatgaaggacgcgcatctggttcagcgtaaaaatgaccacctggatatcgtgctgcaccctgaccgggcgatgagtaccattcgcaccggatttgacgcctggcgttttgaacactgcgccctcccggagctggatctcgacggtatcgatctctccaccaccctgttttcccgcccgctgaaagccccggtgctgatcagctc

SEQ ID NO: 7 - DNA, 756 bp, Artificial Sequence
  Codon-optimized crtW gene from Agrobacterium aurantiacum
caattgaaggaggaataaaccatgagcgcccatgccctgccgaaagccgacctgaccgcgaccagcctgatcgtcagcggtggcatcatcgcggcctggctggcgctgcatgtccatgccctgtggttcctggacgccgccgcccatccgatcctggccatcgccaacttcctgggcctgacctggctgagcgtcggcctgttcatcatcgcgcatgacgccatgcatggcagcgtggtcccgggtcgtccgcgtgccaacgccgccatgggccaactggtcctgtggttgtatgccggcttcagctggcgcaagatgatcgtcaaacatatggcccatcatcgccacgcgggcaccgacgacgatccggacttcgaccatggtggcccggtccgctggtatgcgcgcttcatcggcacctatttcggctggcgtgaaggcctgttgctgccggtcatcgtcaccgtctatgcgctgatcctgggcgaccgctggatgtatgtcgtcttctggccgctgccgagcatcctggcgagcatccaactgttcgtcttcggtacctggctgccgcatcgcccgggccatgacgcctttccggaccgccataacgcccgcagcagccgcatcagcgacccggtcagcctgctgacctgcttccatttcggcggctatcatcatgaacatcatctgcatccgaccgtcccgtggtggcgcctgccgagcacccgcaccaaaggcgacaccgcgtgacaattg

SEQ ID NO: 8 - DNA, 729 bp, Agrobacterium aurantiacum
atgagcgcacatgccctgcccaaggcagatctgaccgccaccagcctgatcgtctcgggcggcatcatcgccgcttggctggccctgcatgtgcatgcgctgtggtttctggacgcagcggcgcatcccatcctggcgatcgcaaatttcctggggctgacctggctgtcggtcggattgttcatcatcgcgcatgacgcgatgcacgggtcggtggtgccggggcgtccgcgcgccaatgcggcgatgggccagcttgtcctgtggctgtatgccggattttcgtggcgcaagatgatcgtcaagcacatggcccatcaccgccatgccggaaccgacgacgaccccgatttcgaccatggcggcccggtccgctggtacgcccgcttcatcggcacctatttcggctggcgcgaggggctgctgctgcccgtcatcgtgacggtctatgcgctgatccttggggatcgctggatgtacgtggtcttctggccgctgccgtcgatcctggcgtcgatccagctgttcgtgttcggcacctggctgccgcaccgccccggccacgacgcgttcccggaccgccacaatgcgcggtcgtcgcggatcagcgaccccgtgtcgctgctgacctgctttcactttggcggttatcatcacgaacaccacctgcacccgacggtgccgtggtggcgcctgcccagcacccgcaccaagggggacaccgcatga

SEQ ID NO: 9 - DNA, 34 bp, Artificial Sequence
  Primer
gacatcgatgtcgaattcgagctcggtaccgatc

SEQ ID NO: 10 - DNA, 32 bp, Artificial Sequence
  Primer
gacctcgtcgctgttattagttgactgtcagc

SEQ ID NO: 11 - DNA, 1302 bp, Methylomonas sp. 16a
atgcaagtttctgtcgagaagacatcagaattaagccgaaaaatgactgttagtgtgcctgatgcggtgcttcaggaaaaaatggaaactcgctttaaaaaactggctcgagaagttaaggttgatggttttcgtcccggtaaagtgccagtcagcacggttaaaaagctttatggcgagcgcgtcaaacacgaagtggccggcgatctgattcaatcgacttatttcgaagcgttgcagcaacaagaattggtgcctgccggccatccgctgatcacgcccgcggacaaaagcgaaggttttgagtatgtggctgaattcgaagtttaccctgaaatcggtctggactcggtcaatggcctggaaatcagtcgtccagtcgctagtgtgaccgacgctgatgtcgaaaacatgatcgaaaagttgaggcagcaaaagaaaacatggcaggtcgtcgaacgggcctcacaagaaggcgacaaagtcaccattcatttctcaggtgtgtctgaaggcgaaaatttcaccgacggcaaggtggaaaattacgcgatcgagatcggcggcaagcagatgattcccggctttgaagacgagctgaaaggcttggccgccggtgaacgcaaaagcttcaacatcacttttccggaaaaatacaacagcgaaaaattggccggcaaagccgctgatttcgagatcgagatgatcaaggtcgaagagccggtattgcctgaactggatgccgacttcatcaaggcctatggtgtagaagcgggcgatgtcgtcagttttcgcgccgacgtcaaggaaaacatggaaagagagttggcgcaaggtctgaaaaacaagctcaaaaccgccgtgttggatgcgctgtacgaaaacgtcaagatcacgctgcccaacgcgttgatcgatcaagaagttcaagcgttgatgaagccttatgcggaacgtgccggcaaggccaagttgaaattggaagatttgaatttgccgcgcgatgtcttcgaagaacaagccaagcgccgcgtggcattgggtttgattttgggcgaaatcatccaaaagaacgacatcaaggtggatgccgataaagtgcgtgccgtgattgacgacatggcgaaaagttacgaaaagccagaagacgttgtaaattggtattatgccgacaatagccgcttaaacgacgttcagcaaatggtgctggaagatcaggccgtggactgggttgtcgagcgtgctcgggttaccgaccaaaatgtcggttttaacgatgtcatggaacggcagcaagcatag

SEQ ID NO: 12 - protein, 433 aa, Methylomonas spl 16a
MQVSVEKTSELSRKMTVSVPDAVLQEKMETRFKKLAREVKVDGFRPGKVPVSTVKKLYGERVKHEVAGDLIQSTYFEALQQQELVPAGHPLITPADKSEGFEYVAEFEVYPEIGLDSVNGLEISRPVASVTDADVENMIEKLRQQKKTWQVVERASQEGDKVTIHFSGVSEGENFTDGKVENYAIEIGGKQMIPGFEDELKGLAAGERKSFNITFPEKYNSEKLAGKAADFEIEMIKVEEPVLPELDADFIKAYGVEAGDVVSFRADVKENMERELAQGLKNKLKTAVLDALYENVKITLPNALIDQEVQALMKPYAERAGKAKLKLEDLNLPRDVFEEQAKRRVALGLILGEIIQKNDIKVDADKVRAVIDDMAKSYEKPEDVVNWYYADNSRLNDVQQMVLEDQAVDWVVERARVTDQNVGFNDVMERQQA

SEQ ID NO: 13 - DNA, 633 bp, Methylomonas sp. 16a
atgattgatctaactggcatgaatactatggttccccaggccgcgggcggtctggtgcccatcgtggtggagcaaacggcgcgcggcgagcggtcgtttgatatttattcccgcttgttgaaagagcgggttatttttctggtgggacaggtcgaagactacatggccaacctggtcgtggctcaattgctgtttctggagtcggaaaatccggacaaggatatacacctgtatatcaattcgcccggcggttccgtgacggcaggcatgtcgatctatgacaccatgcaattcatcaagcccgatgtcagtaccatgtgcatcggtcaggcggccagtatgggggccttgttgttggcgggtggcgcggctggcaagcgttattgcctgccgcattcacgtgtgatgatccatcaacccttgggcggtttccaggggcaggcatcggacatcgccattcatgccaaggaaattttgtccatccgcgataagttgaacaagattctggcgcatcatacggggcagccgttggaaaaaatccagcaagacaccgatagggataattttttgagttccgaacaagcggtcgaatatggtctgatcgataaagttctgagcagccgtaacgtttaa

SEQ ID NO: 14 - protein, 210 aa, Methylomonas sp. 16a
MIDLTGMNTMVPQAAGGLVPIVVEQTARGERSFDIYSRLLKERVIFLVGQVEDYMANLVVAQLLFLESENPDKDIHLYINSPGGSVTAGMSIYDTMQFIKPDVSTMCIGQAASMGALLLAGGAAGKRYCLPHSRVMIHQPLGGFQGQASDIAIHAKEILSIRDKLNKILAHHTGQPLEKIQQDTDRDNFLSSEQAVEYGLIDKVLSSRNV

SEQ ID NO: 15 - DNA, 1272 bp, Methylomonas sp. 16a
atgagtagagacaaaaaaggtaaagacgaagataaacttctttactgttctttttgcggcaaaagccaaaacgaagttagaaagctgattgccgggccgtccgtgtatgtctgcgacgaatgcgtggagctttgtaacgacatcattcgtgacgaattggccgaagacgaaaaaagcgtcggcagcggtgccttgccgaaaccaaaagaaatcaagcaagagctggataattacgtcattggtcaggaaaaggccaagaaaattctatccgtcgcggtttacaaccactataagcggttacgcagcaacagcaaaaaaagcgatgtcgagctggcgaaaagcaacattctgttgatcggcccgaccggctccggtaagacgctgttggcggaaacgctggcacgcttgttggacgtgcctttcacgatcgccgacgccacgacgctgacggaagccggttatgtcggtgaggacgtcgagaatatcattcagaaaattctgcaaaagtgcgattacgatgtcgagaaagcggaaaccggtattgtctacatcgatgaaatcgacaaaatttcacgcaaggccgacaatccatccatcacgcgcgatgtgtccggcgaaggcgtgcagcaagccttgctgaagctgatcgaaggcacggtggcttcagtgccgccgcaaggtggacgcaagcatccgcagcaggaattcttgcaggtgaataccgccaatattctgttcatcgtcggtggcgcttttgccggcttggataaagtcatcaaaaatcgtaccgaaaaaggcggcattggtttttcggcggagctcaaaccgaaggaggagagtcaaaatgtcggtgagatttttgccgatgtgcgcgccgaagacttgatcaaatacggattgatcccggagtttgtcggtcgattgccggtggtggccacgctggacgaattggatgaacacgccttggtgcaaatcctgacccagcctaaaaacgccctgatcaaacaatacaggcatttgttcgaaatggaaggggtggaactggagttcagagaagactccttggcggcgattgcgcgcaaatccatggaaagaaaaaccggcgcgcgtggattgcgcaccatcgtcgaaaacgtgctgctggataccatgtacgaactgccttccagcgacagcatcaccaaggtcgtggtcgacgaaagtgtcatcaatggtcaatccgagccgattctggtgtatgagccggaattgaagcgtgcgtcatccgattga

SEQ ID NO: 16 - protein, 423 aa, Methylomonas sp. 16a
MSRDKKGKDEDKLLYCSFCGKSQNEVRKLIAGPSVYVCDECVELCNDIIRDELAEDEKSVGSGALPKPKEIKQELDNYVIGQEKAKKILSVAVYNHYKRLRSNSKKSDVELAKSNILLIGPTGSGKTLLAETLARLLDVPFTIADATTLTEAGYVGEDVENIIQKILQKCDYDVEKAETGIVYIDEIDKISRKADNPSITRDVSGEGVQQALLKLIEGTVASVPPQGGRKHPQQEFLQVNTANILFIVGGAFAGLDKVIKNRTEKGGIGFSAELKPKEESQNVGEIFADVRAEDLIKYGLIPEFVGRLPVVATLDELDEHALVQILTQPKNALIKQYRHLFEMEGVELEFREDSLAAIARKSMERKTGARGLRTIVENVLLDTMYELPSSDSITKVVVDESVINGQSEPILVYEPELKRASSD

SEQ ID NO: 17 - DNA, 2415 bp, Methylomonas sp. 16a
atgactcaagtaaaaaataaagacgatcttattcctgtgttgccgcttcgggatgtggtggtatatccgcatatggtgatcccgctgttcgtcggccggggaatgtcgatcgatgcgctcgatgccgccatcaaacaggacaaacaagtgctgttgatcgcgcaaaaacaggcggatatcgacgagcccgagtttgacgatttgtataaggtagggacgctggccaatattctgcagttgctcaagttgccggatggcaccgtcaaggtgttggtcgagggaactcagcgttgctcggtcgtgcaatatcaagaatttgaaggctattgcgcggcgggcgtactcgagctggaagatcaaatcacgatcgacgagcaagaactcgacgtattacaacgcacggcgatcaactcgttcgatcaatatgtcaaattgaacaataagattccgccagaagttctgaattctctgtccggtatcgatgatcccggtcgcctggccgacaccatggcggcgcatatggcgctgaaggtggaagaaaaacaaaaaatgctggagatggtcgatgtcgcccggcgcctggaaaatttgatgacgctgatggagggcgaagtcgatatcctggaaatggagaaaaagattcgcggccgggtcaagaaacaaatggaaaaaaaccagcgcgagtattatctgaacgagcaaatgaaggcgattcagaaagagctgggcgagatggacgaagccagcaacgaagtcgaagagctggaaaagaaaatcgaggcggccggcatgtcggccgaagccaaaagcaaagccatggccgagctgaacaagctgaaaatgatgtcgccgatgtcggccgaagcgaccgtcgtgcgcaactatatcgattggatggtcagcgtgccatggaagaaaaaaaccaaggtacgccatgatctcaaggtcgccgaacaggtgctggaatccgaacattacggtctggaaaaggtgaaggaaaggattctggaataccttgcggtacaacaacgggtgaaacaattgaaagggcctattctgtgcctggttgggccgccgggcgtgggtaaaacctcgttgggccagtccatcgcgcgggcgacgaatcgcaaatacgtgcgcatggccttgggcggcgtgcgcgacgaagccgaaatccgcgggcacaggcggacgtatatcggttcgatgccaggcaagattttgcagaatctcgccaagatcaaaacgcgcaatccgatgttcctgctggacgaaatcgacaagatggcggcggattttcgcggcgatccggcgtcggccttgctggaggtgctggatcccgagcaaaatcacaccttcgccgatcattatctggaagtggatttcgatttgtccgacgtgatgttcgtggcaacggcgaataccctgaatattccggcgccgctgatggacagaatggaggtcattcgtctcgcaggttataccgaagacgaaaaaatcaatatcgccacgcgctttttgattcccaagcagatcaaaaacaatggtttggccgaagccgagattgaaattacggaggctgccgtcagggatatgatccgttattacacgcgcgaagccggcgtgcgtagtttggaacgggaaatatcgaagatctgccgcaaagtggtgaaggatttattgctgcataacggtaaagaaaaaatttgtgtcgatagcaaaaacctggataaatatctcggcgtgcgtcgcttcagctatggcatggccgaggatagcgatcagatcggtcaagtgaccggcttggcttggaccgaagtgggcggtgaattgctgactatcgagacggccgtcatgccgggcaaaggcaagcagcaagccaccggtaaactgggcgacgtgatgaaggaatccatcgatgccgccatgacggtggtcaggagtcgtgcggaaatactgggcatagacaaggaggttttccagggtagcgacattcacgttcacgtgccggaaggcgcaacaccgaaggatggtccaagcgcgggcatcgggatgtgtacagcgattatttcggcgctgaccaaaattccggtcagggccgatgtggcgatgaccggcgagattacgctgcgcggtgaggtattgccgattggcggtttgaaagagaagctgttggccgcgcatcgcggcggcatcaggaccgtggtgattccatccgaaaacgaaaaagacttggctgaaatacccgataacgtcaaaagcaatttgaccatcaaatgcgtgcgctggatagacgaggtgttagagatcgcgctgcaaagaatccctgttccgattgcggccgcggaaggtcaaaccgccgaggggcccaatggcaaaaacgaagcgaaaccgcaggcggtgctcgcgcattaa

SEQ ID NO: 18 - protein, 804 aa, Methylomonas sp. 16a
MTQVKNKDDLIPVLPLRDVVVYPHMVIPLFVGRGMSIDALDAAIKQDKQVLLIAQKQADIDEPEFDDLYKVGTLANILQLLKLPDGTVKVLVEGTQRCSVVQYQEFEGYCAAGVLELEDQITIDEQELDVLQRTAINSFDQYVKLNNKIPPEVLNSLSGIDDPGRLADTMAAHMALKVEEKQKMLEMVDVARRLENLMTLMEGEVDILEMEKKIRGRVKKQMEKNQREYYLNEQMKAIQKELGEMDEASNEVEELEKKIEAAGMSAEAKSKAMAELNKLKMMSPMSAEATVVRNYIDWMVSVPWKKKTKVRHDLKVAEQVLESEHYGLEKVKERILEYLAVQQRVKQLKGPILCLVGPPGVGKTSLGQSIARATNRKYVRMALGGVRDEAEIRGHRRTYIGSMPGKILQNLAKIKTRNPMFLLDEIDKMAADFRGDPASALLEVLDPEQNHTFADHYLEVDFDLSDVMFVATANTLNIPAPLMDRMEVIRLAGYTEDEKINIATRFLIPKQIKNNGLAEAEIEITEAAVRDMIRYYTREAGVRSLEREISKICRKVVKDLLLHNGKEKICVDSKNLDKYLGVRRFSYGMAEDSDQIGQVTGLAWTEVGGELLTIETAVMPGKGKQQATGKLGDVMKESIDAAMTVVRSRAEILGIDKEVFQGSDIHVHVPEGATPKDGPSAGIGMCTAIISALTKIPVRADVAMTGEITLRGEVLPIGGLKEKLLAAHRGGIRTVVIPSENEKDLAEIPDNVKSNLTIKCVRWIDEVLEIALQRIPVPIAAAEGQTAEGPNGKNEAKPQAVLAH

SEQ ID NO: 19 - protein, 273 aa, Metylomonas sp. 16a
ATGAATAAATCGGAACTGATCGATGCTATTGCTGCAGCATCTCATTTGACAAAAGCCGATGCCGGCCGTGCTTTAGATGGTTTTATCAAGTCTGTTGAAGAAGCGCTGAAAAAAGGCGATTCAGTAGCCTTGGTTGGTTTTGGCACCTTCGAAGTTAAAGAAAGAGCGGAACGCAAAGGCCGTAATCCGCAAACCGGCGAAGAAATCACTATCAAGGCCGCAAAAATTCCTTCATTTAAAGCTGGCAAATCTTTAAAAGATGCGGTAAACTAG

SEQ ID NO: 20 - protein, 90 aa, Methylomonas sp. 16a
MNKSELIDAIAAASHLTKADAGRALDGFIKSVEEALKKGDSVALVGFGTFEVKERAERKGRNPQTGEEITIKAAKIPSFKAGKSLKDAVN

SEQ ID NO: 21 - DNA, 1869 bp, Methylomonas sp. 16a
atgctattagagattagagaaaaggtgcagggtgtatttgcatcgatcattttagtgctgatttgtgtgctttttggcttatggggtatccagaattatttgggcggcggtaaggaggcccccgtcgttaccgtgggcgataaggatttttttcagcgcgacgttactcaggcctatcagcagtttgctcagaacctggcaggcatgaaattcgacgaagaaacgctgaaaaagcaagccgtgcaaaaactgatacgcgatgaagtgctgttgcaatatgcgcaagaacagaatttggttatcacggacgaaaccgctagaaatttcattcaatcactggaatatttccaaaaagacggccagttcgataagggccagtatcaagcgttgctgagttcgcaaggcatgtcttccgatgatttcgtcaatcgggtgaaaaaggcgttgatgatggaacaggtgcagcatgccgtggtcgagagcggtttcgtgaccaaggctgaaatcgaaagctttttcaagctccaaaatcaaacccgggatgtcgagtatctgacagtacccctggcctcatccagcgaattgccggccgatgacgagataaatgcctattatcaacaacatcaagaggcttaccaaaccccggagcgggccgtcatcagctatgtggaattatccttggatgatttggctaaggatgtcagcgcgtccgatgagcaattgaaagcctattacgaagagcaaaaggcgcaatacactacgcccgaaagacgcaagatcagtcacatcctgtttgcgtttggcaaggacagtgccgatgatcaacaagccttgcaaagagcattgaaggccaagcaagatttggcaaacaaggattttgccgcgttggccgcggaagtatccgatgacaaactgaccgcgaagaatggcggcgatctgggtttgttcaatgtcggcgtgatggaaaaatcctttgaagaggccgctagccagttgaagcaaggcgaagtttcggagccggtcaaatccgcctttggttatcatttgatcaaggtgaccgaattggcgccgggcgagatcaagccttacgaggcggtaaaatcggaattgaccaaggcgtataaaaaaggccaaatcgaaagtcgctttagtgagctgggagaaaaactcagcgaagtgagttacgaaaatccggataatctggatgccgcagcgaagttgcttggggtgccggtcaagacatcggcgccgtttacccgtgatgccggtgaaggtgttgcggtcgatgaaaaagtccgcttggcggctttttccgaagatgtgttgaagggtaataatagcgagccgatcgagattggcagcgagaaattggtggttttgcgcatgcaatctcatacgcctgctgccgccaaggatctcaaggaagtcaaggcggacgtgattgctgcgatacaaaaagagaaggcgctgaagcaagtcgtggaaaaggccgataagctcaaggctgaattggttgccggtaagccgattgcggatgtggcttccgcgactgctttgacggtcaagaaagtggttggcttgagtcgcaatgctggcgatgtggacccggccgtcaggcaggcaattttccgggcggccaagccgcgggcaaatcgtccgagtatcgtggtgatcgatgagcctgctggcggtaaaattgtcgccagtatctctcaagtcaaggaaggtgtcatgactgaggccgacaaggccaagcaagtgcagctggagaaaaatatggcgaccgcatttggcaggacgcaattcgaagccgtgctgaaccagttgcaagccaacgccgatattacgatacgatctcccaagcaataa

SEQ ID NO: 22 - protein, 622 aa, Methylomonas sp. 16a
MLLEIREKVQGVFASIILVLICVLFGLWGIQNYLGGGKEAPVVTVGDKDFFQRDVTQAYQQFAQNLAGMKFDEETLKKQAVQKLIRDEVLLQYAQEQNLVITDETARNFIQSLEYFQKDGQFDKGQYQALLSSQGMSSDDFVNRVKKALMMEQVQHAVVESGFVTKAEIESFFKLQNQTRDVEYLTVPLASSSELPADDEINAYYQQHQEAYQTPERAVISYVELSLDDLAKDVSASDEQLKAYYEEQKAQYTTPERRKISHILFAFGKDSADDQQALQRALKAKQDLANKDFAALAAEVSDDKLTAKNGGDLGLFNVGVMEKSFEEAASQLKQGEVSEPVKSAFGYHLIKVTELAPGEIKPYEAVKSELTKAYKKGQIESRFSELGEKLSEVSYENPDNLDAAAKLLGVPVKTSAPFTRDAGEGVAVDEKVRLAAFSEDVLKGNNSEPIEIGSEKLVVLRMQSHTPAAAKDLKEVKADVIAAIQKEKALKQVVEKADKLKAELVAGKPIADVASATALTVKKVVGLSRNAGDVDPAVRQAIFRAAKPRANRPSIVVIDEPAGGKIVASISQVKEGVMTEADKAKQVQLEKNMATAFGRTQFEAVLNQLQANADITIRSPKQ

SEQ ID NO: 23 - DNA, 26 bp, Artificial Sequence
  Primer
agatctccgttctgtacactgatccg

SEQ ID NO: 24 - DNA, 38 bp, Artificial Sequence
  Primer
agatctgcggcgcccatttgttgctgatagaatccggc

SEQ ID NO: 25 - DNA, 31 bp, Artificial Sequence
  Primer
gcggccgcgcaagccggccaacagggattcc

SEQ ID NO: 26 - DNA, 29 bp, Artificial Sequence
  Primer
gcggccgccgaatacctcgacattcaagc

SEQ ID NO: 27 - DNA, 26 bp, Artificial Sequence
  Primer
agatctaactgtgcgagcgccgtagc

SEQ ID NO: 28 - DNA, 25 bp, Artificial Sequence
  Primer
gcatgccgacatctagttgtccagc

SEQ ID NO: 29 - DNA, 28 bp, Artificial Sequence
  Primer
agatcttggcgcttgatcgaaatcgtcg

SEQ ID NO: 30 - DNA, 28 bp, Artificial Sequence
  Primer
gcggccgctgtcgtgcgaatgcatcagc

SEQ ID NO: 31 - DNA, 29 bp, artificial
  primer
agatctttgcaacgggtattcgacgaagg

SEQ ID NO: 32 - DNA, 39 bp, artificial
  primer
gcatgcctcgagtgctatcgtcgtcatactcaggctttg

SEQ ID NO: 33 - protein, 242 aa, Agrobacterium aurantiacum
MSAHALPKADLTATSLIVSGGIIAAWLALHVHALWFLDAAAHPILAIANFLGLTWLSVGLFIIAHDAMHGSVVPGRPRANAAMGQLVLWLYAGFSWRKMIVKHMAHHRHAGTDDDPDFDHGGPVRWYARFIGTYFGWREGLLLPVIVTVYALILGDRWMYVVFWPLPSILASIQLFVFGTWLPHRPGHDAFPDRHNARSSRISDPVSLLTCFHFGGYHHEHHLHPTVPWWRLPSTRTKGDTA

SEQ ID NO: 34 - DNA, 22 bp, artificial
  primer
tgcccggtgccagcgtgccttc

SEQ ID NO: 35 - DNA, 950 bp, Pantoea stewartii
gaattcgcccttgacggtctgcgcaaaaaaacacgttcaccttactggcatttcggctgagcagttgctggctgatatcgatagccgccttgatcagttactgccggttcagggtgagcgggattgtgtgggtgccgcgatgcgtgaaggcacgctggcaccgggcaaacgtattcgtccgatgctgctgttattaacagcgcgcgatcttggctgtgcgatcagtcacgggggattactggatttagcctgcgcggttgaaatggtgcatgctgcctcgctgattctggatgatatgccctgcatggacgatgcgcagatgcgtcgggggcgtcccaccattcacacgcagtacggtgaacatgtggcgattctggcggcggtcgctttactcagcaaagcgtttggggtgattgccgaggctgaaggtctgacgccgatagccaaaactcgcgcggtgtcggagctgtccactgcgattggcatgcagggtctggttcagggccagtttaaggacctctcggaaggcgataaaccccgcagcgccgatgccatactgctaaccaatcagtttaaaaccagcacgctgttttgcgcgtcaacgcaaatggcgtccattgcggccaacgcgtcctgcgaagcgcgtgagaacctgcatcgtttctcgctcgatctcggccaggcctttcagttgcttgacgatcttaccgatggcatgaccgataccggcaaagacatcaatcaggatgcaggtaaatcaacgctggtcaatttattaggctcaggcgcggtcgaagaacgcctgcgacagcatttgcgcctggccagtgaacacctttccgcggcatgccaaaacggccattccaccacccaactttttattcaggcctggtttgacaaaaaactcgctgccgtcagttaacaattgagtgggcaggattatgcaaccg

SEQ ID NO: 36 - DNA, 3611 bp, Pantoea stewartii
aaaactcgctgccgtcagttaacaattgagtgggcaggattatgcaaccgcactatgatctcattctggtcggtgccggtctggctaatggccttatcgcgctccggcttcagcaacagcatccggatatgcggatcttgcttattgaggcgggtcctgaggcgggagggaaccatacctggtcctttcacgaagaggatttaacgctgaatcagcatcgctggatagcgccgcttgtggtccatcactggcccgactaccaggttcgtttcccccaacgccgtcgccatgtgaacagtggctactactgcgtgacctcccggcatttcgccgggatactccggcaacagtttggacaacatttatggctgcataccgcggtttcagccgttcatgctgaatcggtccagttagcggatggccggattattcatgccagtacagtgatcgacggacggggttacacgcctgattctgcactacgcgtaggattccaggcatttatcggtcaggagtggcaactgagcgcgccgcatggtttatcgtcaccgattatcatggatgcgacggtcgatcagcaaaatggctaccgctttgtttataccctgccgctttccgcaaccgcactgctgatcgaagacacacactacattgacaaggctaatcttcaggccgaacgggcgcgtcagaacattcgcgattatgctgcgcgacagggttggccgttacagacgttgctgcgggaagaacagggtgcattgcccattacgttaacgggcgataatcgtcagttttggcaacagcaaccgcaagcctgtagcggattacgcgccgggctgtttcatccgacaaccggctactccctaccgctcgcggtggcgctggccgatcgtctcagcgcgctggatgtgtttacctcttcctctgttcaccagacgattgctcactttgcccagcaacgttggcagcaacaggggtttttccgcatgctgaatcgcatgttgtttttagccggaccggccgagtcacgctggcgtgtgatgcagcgtttctatggcttacccgaggatttgattgcccgcttttatgcgggaaaactcaccgtgaccgatcggctacgcattctgagcggcaagccgcccgttcccgttttcgcggcattgcaggcaattatgacgactcatcgttgaagagcgactacatgaaaccaactacggtaattggtgcgggctttggtggcctggcactggcaattcgtttacaggccgcaggtattcctgttttgctgcttgagcagcgcgacaagccgggtggccgggcttatgtttatcaggagcagggctttacttttgatgcaggccctaccgttatcaccgatcccagcgcgattgaagaactgtttgctctggccggtaaacagcttaaggattacgtcgagctgttgccggtcacgccgttttatcgcctgtgctgggagtccggcaaggtcttcaattacgataacgaccaggcccagttagaagcgcagatacagcagtttaatccgcgcgatgttgcgggttatcgagcgttccttgactattcgcgtgccgtattcaatgagggctatctgaagctcggcactgtgccttttttatcgttcaaagacatgcttcgggccgcgccccagttggcaaagctgcaggcatggcgcagcgtttacagtaaagttgccggctacattgaggatgagcatcttcggcaggcgttttcttttcactcgctcttagtgggggggaatccgtttgcaacctcgtccatttatacgctgattcacgcgttagaacgggaatggggcgtctggtttccacgcggtggaaccggtgcgctggtcaatggcatgatcaagctgtttcaggatctgggcggcgaagtcgtgcttaacgcccgggtcagtcatatggaaaccgttggggacaagattcaggccgtgcagttggaagacggcagacggtttgaaacctgcgcggtggcgtcgaacgctgatgttgtacatacctatcgcgatctgctgtctcagcatcccgcagccgctaagcaggcgaaaaaactgcaatccaagcgtatgagtaactcactgtttgtactctattttggtctcaaccatcatcacgatcaactcgcccatcataccgtctgttttgggccacgctaccgtgaactgattcacgaaatttttaaccatgatggtctggctgaggatttttcgctttatttacacgcaccttgtgtcacggatccgtcactggcaccggaagggtgcggcagctattatgtgctggcgcctgttccacacttaggcacggcgaacctcgactgggcggtagaaggaccccgactgcgcgatcgtatttttgactaccttgagcaacattacatgcctggcttgcgaagccagttggtgacgcaccgtatgtttacgccgttcgatttccgcgacgagctcaatgcctggcaaggttcggccttctcggttgaacctattctgacccagagcgcctggttccgaccacataaccgcgataagcacattgataatctttatctggttggcgcaggcacccatcctggcgcgggcattcccggcgtaatcggctcggcgaaggcgacggcaggcttaatgctggaggacctgatttgacgaatacgtcattactgaatcatgccgtcgaaaccatggcggttggctcgaaaagctttgcgactgcatcgacgcttttcgacgccaaaacccgtcgcagcgtgctgatgctttacgcatggtgccgccactgcgacgacgtcattgacgatcaaacactgggctttcatgccgaccagccctcttcgcagatgcctgagcagcgcctgcagcagcttgaaatgaaaacgcgtcaggcctacgccggttcgcaaatgcacgagcccgcttttgccgcgtttcaggaggtcgcgatggcgcatgatatcgctcccgcctacgcgttcgaccatctggaaggttttgccatggatgtgcgcgaaacgcgctacctgacactggacgatacgctgcgttattgctatcacgtcgccggtgttgtgggcctgatgatggcgcaaattatgggcgttcgcgataacgccacgctcgatcgcgcctgcgatctcgggctggctttccagttgaccaacattgcgcgtgatattgtcgacgatgctcaggtgggccgctgttatctgcctgaaagctggctggaagaggaaggactgacgaaagcgaattatgctgcgccagaaaaccggcaggccttaagccgtatcgccgggcgactggtacgggaagcggaaccctattacgtatcatcaatggccggtctggcacaattacccttacgctcggcctgggccatcgcgacagcgaagcaggtgtaccgtaaaattggcgtgaaagttgaacaggccggtaagcaggcctgggatcatcgccagtccacgtccaccgccgaaaaattaacgcttttgctgacggcatccggtcaggcagttacttcccggatgaagacgtatccaccccgtcctgctcatctctggcagcgcccgatctaggtacc

SEQ ID NO: 37 - DNA, 20 bp, Artificial Sequence
  Primer
gaattcgcccttgacggtct

SEQ ID NO: 38 - DNA, 50 bp, Artificial Sequence
  Primer
cggttgcataatcctgcccactcaattgttaactgacggcagcgagtttt

SEQ ID NO: 39 - DNA, 50 bp, Artificial Sequence
  Primer
aaaactcgctgccgtcagttaacaattgagtgggcaggattatgcaaccg

SEQ ID NO: 40 - DNA, 25 bp, Artificial Sequence
  Primer
ggtacctagatcgggcgctgccaga

SEQ ID NO: 41 - DNA, 8814 bp, Pantoea agglomerans
accgcgaagacagcaacgtgctggagacccgctttgagacggaaccgggtcggtgctgatcaccgagtcgctgaacagcacgcttgctggccggctgccgtggagcgaactggcccgccgcatcgacggtattgagggccacgtgacgctgaacgtcagcctgcgtttcggtaccgctgccgagacgcgctccccgtggagggcgaacacctttaagggcgatgtgtttcacattgccgatctgatggcgatgctgcgcaccagcgaagacattgagattacccactgcgacgatgaaaaaattaccgcccagctgatgacctcaccggggtcgcgctcgctggtcgccctgctggtcaccgagaaagagccgctggcggtgccggatctctccgccatcgatgaccgcatcgaaaccagccaccttgcctggtgcgactggacccgcagcctcagctaccgcggtctctacgacaagcacgtcaaacgatccgcgctggcgctgaagtttctctggtactccccgaccggcgcgctggcggcggcggccaccacctcgctgccggaaggcattggcggggagaaaaactacgactaccgctatgcctgggtgcgcgatgcctgtctgatcatcaaagcgttcgtgttcctcggtgcgctggaggactgcaaagccgccttctcctggctgtcgaaaaccattattcgccacgggcctgagctgcgcgcctgctatacgctcgaaggtgacgaggtgccggccgagtactatccgccgctgcgcggatatcgggattcccgcccggtgcgggtgggcaacaacgcccgcaaccagatccagctcagcatgtacggcgacatgctcgccaccgcgcagctgtttatcgaggcgggacacgtactggatctcgccacctcgcgcctgcttggcgaactggcgaactgctgcgccgacagctggcggcagaaggactccggcatctgggagttaccggacgagcagcactatacccactcgaagatggcctgctggctggcactggatcgcgccgtggcgatggcagaacagaagcacatcgaaccgacctgggtcgggcgctggcagcgcgagcgcgatcggatccgcgactggatcgaaacccactgctggtcggagaaaaagcaggcctacgtgttttacgtcggggacgacgagcggctggatgccgcgctggcgctggtgcacgactacggcaacagcgtaaacccgcagcgtatgctggccacctatcgcgccatcaaagcggagctgggacacgacacgcccatgctctaccgctacagcgaggtggaaaaggaagaaagcacctttgtcgcctgctcgttctggctggtggaagccctcgccgcgatgggtgaaaccgacgaggcccaggcggccatgaccggcatcctcgagaggctctgcgaccggggcaatgttgaaacttttaacgagatgtttgatgtgcgtaccgacgagtggcgcggcaaccttcctcaggggctgagccatctggcgctgatctgcgccgcgcaggcgctttcggaaaaatgccgcaacacgcgcgactgacgcacgcgtagctaaggagaagacgatgaccatcagaggtatcgaacatattggtattaccgtcgccgacctcaccggggcggagcggtttttcatcgaggcgctggatgccagcgtgctctaccgcatcgtgccgcccggcgcggcggacaatgccatcagcggcgaccagatgacgcggctcaatggctttcccccggagatgcgggttaccggcctggccatgctgcgtctcggcaacggctgcaatattgagctgtttgagatcgatcccggcgtggcagacgcgcccggaaatatcagccaggcgggcctgaaccacctgtcggtttacgtggacgacattcagcaggccggcgcacgggtaaaagcacagggcgccacgctgtttgacgggccgagcgactgctttgctcaggaagagggccgcggcaaccagacctggttctgccgcacgccttttggcctgctgattgaactcatctcccttccctcgccgcttcgctacgatgcgcaggcgcagcaaacccgctggatcccccagcgctgacaggcctctctcacgcgggcatcgcccgcgttgtcataccctcgtcaccgtcctgacaaaaattaacaataaattttcatttttcagccagactttaagcacatagcgtcgccatgacatttattttcatctaaacctatacaagaaaaacattgatgtataactttgcataccgctgcacacaggctcagactgcgacacccgttgcgggtcagcgctatttccatttcatctgcgagacgccgggtaccaaccatgacaagaccctttgaaacacatcccggtcacgacggggaactgcatgagctgcacgctgccctgcaacgtcgcctggatgaactgctgcccgttggcgatgagcgggatcgggtcagcagcgcaatgcgcgaaggcgtactggcaccggggaaacgcattcgcccgctgctcctgatcctcgccgcccgcgacctcggctgcgatcgcgaccaccccggcctgctggatatggcctgtgcggtggaaatggtgcacgcctcgtcgctgatcctcgacgatattccctgcatggataacgcggcgctccggcgcggtcgccctaccattcatcgccagtatggtgaagacgtggcaattctcgctgcggtagcgttgctcagcagcgcctttggcgtgatggtcgcggcgcagggattgtctcccgagtgccgcagccaggcggtggcggagctgtcgatggcggtcggtacccagggtctggtgcagggtcagtataaggatctgcgtgaaggcaccgccccgcgcagcgccgaggagatcgccaccaccaacgaactgaaaaccagcgtgctgtttggtgccacgctgcaaatcgcggccctggcggcaggcgcctcgccggcggcgcgccagaaaatgcgctgctttgcgcaggatttaggccaggcgttccagctgctggacgatctggcggacggccatgccgggaccggcaaagacatcaataaggacgcgggtaagtccacgctggtggcgatgctcggcagcgacgcggtgcgcgagcggctcgacacccatctgcgccgcgcagacgcccatttttcacgcgcctgcggaaaaaaccaggccacgcgacgctttatgcacgcctggttttcaaaacagctggccgcgtttagctgagcaacggatacaccccggtaatatttgtggagatcacatgaaggacgcgcatctggttcagcgtaaaaatgaccacctggatatcgtgctgcaccctgaccgggcgatgagtaccattcgcaccggatttgacgcctggcgttttgaacactgcgccctcccggagctggatctcgacggtatcgatctctccaccaccctgttttcccgcccgctgaaagccccggtgctgatcagctccatgaccggcggcgcggcgcgcgccagagacattaaccgtcatctggcccaggcggcgcaaacccttgggctggcgatgggcgtcggttcccagcgcgtggcgctggaggacggcgcgcagcacgggctggatgcccagctacgccatatcgccccggacgtgccgctgctggctaaccttggcgcggcgcagatccgcggtgcgcaggggctggactacgcccggcgcgcggtggacatgatcgacgccgacgcgttaattgtgcatctgaacccgctgcaggaggcgctccagggcggcggcgatcgcgactggcgcggcatcctcaacgccattgcgcagctggtgcgcgacctgccggtaccggtggtggttaaagaggtgggcgccgggatctccccggacgttgcctgccgactggcggacgtcggcgtggcgatgatcgacattgccggcgcgggcggaaccagctgggcggcggtggaagctgaacgcgccccgacccccgaggcgcgaaatgtggcgatggcctttgccgactggggcattcctactgccgatgcgctgcgtcgcgtccatcttgcgctgcctgatatcccgcttatcgcctccggcggcatcgccaacggcattgacgcagcaaaagccatcgcgctgggtgcagatctggtgggccaggccgcggcggtgctggcgcatgccaacgcctccggcgacgcggcaattgcccatttccgcaccctgattacgcagctgcggatcgcctgtttctgtaccggcagtgcaaacctgcaggcgttgcgacacgccacgctgcttccggtcaacggcggcgcatccctgtgacgcagtacggtgccttataccggggagcggtatgaaaaaatgggatctgattctggtcggcgcggggctggccaacgggcttatcgcctggcgactaaagcagcgtcatccgacgcttgctgtattaatgctggagtgcggcgacgcgcccggcggaaaccacacctggtcctttcaccaacacgatatcacgccagcccagcacgcctggctggcgccgctggtggcccatcgctgggacgggtacgacgtccactttccgaacgtgtcgcgcaccctgcatgacggctacctgaccatcacctccacgcgttttgcccaagcgatgcgcgggctgatgaaagagaatttgctgacaaacgtgaccgtgtcacgggtgagcgggcaggaagtaaccctcagcgacggacgacgctttaccgccggggcggtgattgatggccgcggctatcagccctcgccgcacctcagcattggctatcaggcgttcatcggccaggagtggcaactgaccgcgccccacgggttaacgcgcccgatcctgatggatgcccgcgtcgcccagggcaacggctaccgctttgtctataccctgccgctcagcgccgacaccctgcttatcgaagacacgcactacattgacggcccgacgctcgacgccgattcagcccgcgcgcggattgccgattacgcccgccagcagggctggcagcttgcgcggctggtgcgtgaggaacagggggcgctgccgatcaccctgtccggcgatccggccgccttctggcaccagttccatcatcagccggtcagcggcctgcgcgccggtctgttccatgccaccaccggctattcgctgccgctggcggttcggctggcggaccgcattgccaacgcgccgggactgcatcagggcgcgctctatcagctgatcgccgatttcgcggcgcgccactggcagacacaacgctttttccgcctgcttaaccgcatgcttttcctggccggcacacccgaccagcgctggcgcgtgatgcagcggttttaccagcttgacgagcagctgatcgcccgtttttatgccggccagcttcgctccgccgaccgcgcgcgcctgctgcttggcaaaccgccggtgccgattgtcggggcgatcaaagccctgctccacactcattcttctctgcgagcccatcataaatgaaacaaaccattgtaattggcgccgggttcggcggactggcgctggcgattcgcctccaggcggcgggcattcctaccacgctgctggagagccgcgacaaacccggcggccgcgcctatgtctacgaagatcgcggctttacctttgatgcgggtcccaccgtcatcaccgatccctccgccattgaggagctgttcaccctcgccggaaaacggctgaaggactacgttgagctgatgccggtgacgccgttctatcgcctgtgctgggaagacggcaaggttttcgactacgccaacgatcaggcggcgcttgagtcgcagatcgccgcgtttaacccgaacgacgtggcgggctatcaccgcttcctcgactactcccgggcggtgtttgccgaaggctatctgaagctcggcgcggtgccgtttctctcgtttcgcgacatgctgcgcgccggtcctcaactggcgcggctgcaggcatggcgcagcgtgtacgacaaagtgtcggcctacgtggaagacgagcacctgcggcaggcattttcgtttcactcgctgctggtgggcggcaacccgttctccacgtcttctatttacaccctgatccacgccctggagcgggaatggggcgtctggttcccgcgcggcggcaccggtgcgctggttcagggcatggtgaagctgtttcaggatcttggcggcaccctcacccttaacgctcaggttgagcggctggagacggtggacaatcaggtgaaggccgtgcatctggttaacgggcagcggctggaggctgcggcggtggcctcgaacgcggacgtggtaaatacctatgcccgactgctcggccatcacccgcacggcgccgctacggccaaaaagctgaaacgcaagcgcatgagcaactcgctgttcgtgctctattttggcctggatcaccatcacacccagctggcgcaccataccgtctgctttggcccgcgttataaagcgctaatcgatgaaattttcagcgccgacaccctgtcggaagatttttcgctctatctgcatgcgccctgcgtaaccgacccgtcgctggccccgccggggtgcggcagctactatgtgctcgcgccggtgccgcacctcggtaacgccccgctcgactggagcgtggaagggccgcgtctgcgggatcgcatttttgattatctcgaagcgcgctatatgccggggctgcgctcccagctggtgacgcaccgcatgttcacgccggaagattttcgcgatacgctcgatgcctggcaggggtcagcgttttcactggagccgatcctcacccagagcgcctggttccggccgcacaaccgcgacagcgtggttgataacctctacctggtcggcgccggaacgcatcccggcgctggcgtgccgggcgtgatcggatccgccaaggcaacggcccagttaatgttaaaggatttagcgtaatgtcccagccgcttctcgaacacgccagcgccaccatgaccgccggttctaaaagtttcgccaccgcctcaaagctgtttgacaaacgcacccggcgcagcgcgctgatgctctatacctggtgccgctactgcgacgatgttatcgacggacaggtggtgggttttgctgccccgaccgagcagagcgacacgcccgaggcgcgcctgcaacggctgcgtaagatgacgcgccgcgcctacgacggggaaaccatgcaagagccgccgttcgccgcctttcaggaggttgccctcgcccatgccattccgcctactcaggccttcgaccacctggaaggctatgcgatggacgtgcgcaacgagcgctattacagcctcgatgatacgctccgctactgttatcacgtggcgggcgtggtcggcctgatgatggccagggtgatgggagtgcgggacgaagccacgctggatcgcgcctgcgatctgggcattgcctttcagctcaccaatatcgccagggatatcgttgacgatgcgcaggtgggacgctgctacctgccgcagcagtggctggcggaagtcggactcaatgaacagacctgcaccgtgcgggccaaccgtccggcgctggcgcgtctggcagcgcggctggtgaccgaggctgagccctattatcagtcagcgcttgccgggctgggggatctgcccctgcgctccgcctgggcgattgccaccgcgcacggggtgtatcgtgagatcggggtgaaggtgctgatggcgggtgaaaaagcatgggatacccgccagggcacgacgcgcgcggagaagctggcgctggttatttccggcgcgaagcaggcgatggcttcccggaaggcgagctggccgccgcgcgatccgcacctctggcagcgcccgcgctagcgggtctgccgttacgttcgcgcagcaccgcctgcagcttgtccaccggtggggcgtagataaacccgaaggagacgcacccttcgcgcccccgcaccgcgtgatgcagccggtgtgccatgtagaggcggcgcagatagccgcggcgcggcacgtaacggaacggccagcgctggtggactaaaccatcgtgaacgataaagtagatcacgccgtagccggtcattcccgcgccaatccactgaagcggccagtacccttcgctgcccgcgtaaatcagcgcaatggccagtagcgcaaacaccaccgcatagagatcgttacgctcaaacgcccctttgcgcggggtatggtgcgaatgatgccagccccatccccagccgtgcatgatgtacttgtgtgcgaacgttgccaccccttccatgatgatgatagtcagtagcacgatcccggtattccacaacgcaagcataggtttttcctgtagttgacagcccctaaagcgtagcctggaatgccaggaaacataagcgtaacctcggggataatgcgcttttcaggcgtaaaagcatttatgacaattattcatcgcgccacgttcacgccgtgacgccctgctcaccgcgcggcagcagccgcatcggctgataaacgcgcccggtttctgcgcgtcatcgcccggtgtgcgcggcgtcaacgcaataaaacttactttcaaaaggcggcccgaaaaggctacccttttttattcttgtcatatactcgatctaacctgaattatcgccgtaacgtaccgcttcttttgaggtaatcccggagc

SEQ ID NO: 42 - DNA, 34 bp, Artificial Sequence
  Primer
gaattcactagtcgagacgccgggtaccaaccat

SEQ ID NO: 43 - DNA, 25 bp, Artificial Sequence
  Primer
gaattctagcgcgggcgctgccaga

SEQ ID NO: 44 - DNA, 80 bp, artificial
  linker
atccaattggcggccgcgactagttctagacgaattcagatctttaattaaggatccggtaccgcggccgccaattgatc

SEQ ID NO: 45 - DNA, 80 bp, artificial
  linker
gatcaattggcggccgcggtaccggatccttaattaaagatctgaattcgtctagaactagtcgcggccgccaattggat

SEQ ID NO: 46 - DNA, 38 bp, artificial
  primer
gactagtagatcttctgattagaaaaactcatcgagca

SEQ ID NO: 47 - DNA, 39 bp, artificial
  primer
gactagtggatccggaaagccacgttgtgtctcaaaatc

SEQ ID NO: 48 - DNA, 42 bp, artificial
  primer
ccttaattaaggatccgatcttaacatttttcccctatcatt

SEQ ID NO: 49 - DNA, 39 bp, artificial
  primer
ccttaattaagatctgttattagttgactgtcagctgtc

SEQ ID NO: 50 - DNA, 36 bp, artificial
  primer
atttgcggccgccatacgagccggaagcataaagtg

SEQ ID NO: 51 - DNA, 36 bp, artificial
  primer
atttgcggccgcctgattaataagatgatcttcttg

SEQ ID NO: 52 - DNA, 24 bp, artificial
  primer
agtaactgatcaaggcggctatcg

SEQ ID NO: 53 - DNA, 23 bp, artificial
  primer
tatcgatatcagccagcaactgc

SEQ ID NO: 54 - DNA, 32 bp, artificial
  primer
gctctagagaagtttaccctgaaatcggtctg

SEQ ID NO: 55 - DNA, 31 bp, artificial
  primer
gaattcttcctatgcttgctgccgttccatg

SEQ ID NO: 56 - DNA, 57 bp, artificial
  primer
catggaacggcagcaagcataggaagaattcactgaatgattgatctaactggcatg

SEQ ID NO: 57 - DNA, 33 bp, artificial
  primer
gaagatctgctgcggatgcttgcgtccaccttg

SEQ ID NO: 58 - DNA, 21 bp, artificial
  primer
gcccgcggacaaaagcgaagg

SEQ ID NO: 59 - DNA, 21 bp, artificial
  primer
agttacttcccggatgaagac

SEQ ID NO: 60 - DNA, 20 bp, artificial
  primer
aacagaatattggcggtatt